(19) United States
(12) Patent Application Publication
(10) Pub. No.: US 2014/0335562 A1
(43) Pub. Date: Nov. 13, 2014

(54) Genetically Engineered Herbicide Resistant Algae

(71) Applicant: Sapphire Energy, Inc., San Diego, CA (US)

(72) Inventors: Su-Chiung FANG, Sinshih Township (TW); Yan POON, San Diego, CA (US); Michael MENDEZ, San Diego, CA (US)

(73) Assignee: SAPPHIRE ENERGY, INC., San Diego, CA (US)

(21) Appl. No.: 14/329,415

(22) Filed: Jul. 11, 2014

Related U.S. Application Data
(63) Continuation of application No. 13/142,917, filed on Sep. 4, 2012.
(60) Provisional application No. 61/142,091, filed on Dec. 31, 2008.

(51) Int. Cl. C12N 15/79

(52) U.S. Cl. CPC C12N 15/79
USPC 435/69.1; 435/257.2; 435/471; 435/183

(57) ABSTRACT

Algae transformed with one or more polynucleotides encoding proteins that confer herbicide resistance are provided. The algae can be grown in small or large scale cultures that include one or more herbicides for the production and isolation of various products.

### CROSS REFERENCE TO RELATED APPLICATION

This application claims the benefit of U.S. Provisional Application No. 61/142,091, filed Dec. 31, 2008, the entire contents of which are incorporated by reference for all purposes.

### INCORPORATION BY REFERENCE

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

### BACKGROUND

Algae are highly adaptable plants that are capable of rapid growth under a wide range of conditions. As photosynthetic organisms, they have the capacity to transform sunlight into energy that can be used to synthesize a variety of useful compounds. The present disclosure recognizes that large scale cultures of algae can be used to produce a variety of biomolecules for use as industrial enzymes, therapeutic compounds and proteins, nutritional products, commercial products, or fuel products, for example. The disclosed methods, polynucleotides, and algae can be used for the large-scale production of useful compounds as well as for other purposes, such as, for example, carbon fixation, or the decontamination of compounds, solutions, or mixtures.

The present disclosure also recognizes the potential for algae, through photosynthetic carbon fixation, to convert CO₂ to sugar, starch, lipids, fats, or other biomolecules, for example, thereby removing a greenhouse gas from the atmosphere, while providing therapeutic or industrial products, for example, a fuel product, or nutrients for human or animal consumption.

To allow for the large scale growth of algal cultures in open ponds or large containers, for example, in which the algae efficiently and economically have access to CO₂ and light, it is important to deter the growth of competing organisms that might otherwise contaminate and even overtake the culture.

Provided herein are algae transformed with nucleic acid sequences that confer herbicide resistance to the algae. The herbicide resistant algae are then able to grow in the presence of the herbicide at a concentration that deters growth of algae not harboring the herbicide resistance gene. The presence of the herbicide may also deter the growth of other organisms, such as, but not necessarily limited to, other algal species.

### SUMMARY

Provided herein are isolated polynucleotides for transformation of an alga, wherein the polynucleotide comprises one or more nucleic acid sequences encoding a protein that confers herbicide resistance to the alga, wherein the nucleic acid sequence comprises: (a) the nucleotide sequence of SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO:100; (b) a nucleotide sequence homologous to SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO: 100; or (c) the nucleotide sequence of SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO: 100, comprising one or more mutations.

In one aspect, the nucleic acid sequence encoding the protein is codon biased to reflect the codon bias of the nuclear genome of the alga. In another aspect, the nucleic acid sequence encoding the protein is codon biased to reflect the codon bias of the nuclear genome of Chlamydomonas reinhardtii.

In yet another aspect, the nucleic acid sequence encoding the protein is codon biased to reflect the codon bias of the chloroplast genome of the alga. In other embodiments, the alga can be a eukaryotic alga or a prokaryotic alga.

In some embodiments, the polynucleotide is a heterologous polynucleotide, the polynucleotide is a homologous polynucleotide, or the polynucleotide is a homologous mutant polynucleotide.

In one embodiment, the polynucleotide further comprises a promoter operably linked to the sequence encoding the protein. In yet another embodiment, the polynucleotide further comprises a promoter for expression in the nucleus of Chlamydomonas reinhardtii. In some embodiments, the polynucleotide further comprises a rbcS promoter, an LHCP promoter, or a nitrate reductase promoter. In one embodiment, the polynucleotide further comprises a chloroplast transit peptide-encoding sequence.

In one embodiment, the herbicide is glyphosate.

Also provided herein are isolated polynucleotides for transformation of an alga, wherein the polynucleotide comprises one or more nucleic acid sequences encoding a protein that confers herbicide resistance to the alga, wherein the protein comprises: (a) the amino acid sequence of SEQ ID NO: 1. SEQ ID NO: 2, SEQ ID NO: 3, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; (b) an amino acid sequence homologous to SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; or (c) the amino acid sequence of SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; comprising one or more mutations.

In one embodiment, the nucleic acid sequence encoding the protein is codon biased to reflect the codon bias of the nuclear genome of the alga. In another embodiment, the nucleic acid sequence encoding the protein is codon biased to reflect the codon bias of the nuclear genome of Chlamydomonas reinhardtii. In yet another embodiment, the nucleic acid sequence encoding the protein is codon biased to reflect the codon bias of the chloroplast genome of the alga.

In other embodiments, the alga can be a eukaryotic alga or a prokaryotic alga.

In other embodiments, the polynucleotide is a heterologous polynucleotide, the polynucleotide is a homologous polynucleotide, or the polynucleotide is a homologous mutant polynucleotide.

In one embodiment, the polynucleotide further comprises a promoter operably linked to the sequence encoding the protein. In another embodiment, the polynucleotide further comprises a promoter for expression in the nucleus of Chlamydomonas reinhardtii. In other embodiments, the polynucleotide further comprises a rbcS promoter, an LHCP promoter, or a nitrate reductase promoter.

In one embodiment, the polynucleotide further comprises a chloroplast transit peptide-encoding sequence.

In yet another embodiment, the herbicide is glyphosate.

Provided herein are herbicide resistant alga comprising a recombinant polynucleotide integrated into the alga genome, wherein the recombinant polynucleotide comprises a sequence encoding one or more proteins that confer herbicide resistance to the alga.

In some embodiment, the alga may be a prokaryotic alga or a eukaryotic alga.

In one embodiment, the herbicide is glyphosate.

In other embodiments, the protein is a homologous 5-enolpyruvylshikimate-3-phosphate synthase (EPSPS), the protein is a homologous mutant 5-enolpyruvylshikimate-3-phosphate synthase (EPSPS), or the protein is a heterologous 5-enolpyruvylshikimate-3-phosphate synthase (EPSPS).

In one aspect, the polynucleotide comprises one or more of: (a) the nucleotide sequence of SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO:100; (b) a nucleotide sequence homologous to SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO: 100, or (c) the nucleotide sequence of SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO: 100, comprising one or more mutations.

In another aspect, the protein comprises one or more of: (a) the amino acid sequence of SEQ ID NO: 1, SEQ ID NO: 2, SEQ ID NO: 3, SEQ ID NO: 4, SEQ ID NO: 6, SEQ ID NO: 7, SEQ ID NO: 9. SEQ ID NO:11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; (b) an amino acid sequence homologous to SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; or (c) the amino acid sequence of SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; comprising one or more mutations.

Also provided herein are glyphosate resistant eukaryotic alga comprising a recombinant polynucleotide integrated into the nuclear genome, wherein the recombinant polynucleotide comprises a sequence encoding a 5-enolpyruvylshikimate-3-phosphate synthase (EPSPS) that confers glyphosate resistance to the alga.

In some embodiments, the recombinant polynucleotide encodes a homologous EPSPS, the recombinant polynucleotide encodes a homologous mutant EPSPS, or the recombinant polynucleotide encodes a heterologous EPSPS protein.

In one embodiment, the sequence encoding the EPSPS is codon biased to reflect the codon bias of the nuclear genome of the alga.

In another embodiment, the sequence encoding the EPSPS is operably linked to a promoter that functions in the nucleus of the alga. In other embodiments, the promoter that functions in the nucleus of the alga comprises a 16SrRNA promoter, an rbcL promoter, an atpA promoter, a psaA promoter, a psbA promoter, or a psbD promoter. In some embodiments, the sequence encoding the EPSPS is operably linked to a 5′ UTR that functions in the nucleus of the alga or the sequence encoding the EPSPS is operably linked to a 3′ UTR that functions in the nucleus of the alga. In yet another embodiment, the recombinant polynucleotide further comprises a transcriptional regulatory sequence for expression of the polynucleotide in the nucleus of the alga.

In one embodiment, the alga is a non-chlorophyll c-containing eukaryotic alga. In another embodiment, the alga is green alga. In some embodiments, the green alga is a Chlorophycean, Chlamydomonas, Scenedesmus, Chlorella, or Nannochlorpis. In one embodiment, the Chlamydomonas is C. reinhardtii. In another embodiment, the Chlamydomonas is C. reinhardtii 137c. In one embodiment, the alga is a microalga. In other embodiments, the microalga is a Chlamydomonas, Volvacales, Dunaliella, Scenedesmus, Chlorella, or Hematococcus species. In yet another embodiment, the alga is a macroalga.

Also provided herein are glyphosate resistant eukaryotic alga comprising a recombinant polynucleotide integrated into the chloroplast genome, wherein the recombinant polynucleotide comprises a sequence encoding a 5-enolpyruvylshikimate-3-phosphate synthase (EPSPS) that confers glyphosate resistance to the alga.

In some embodiments, the recombinant polynucleotide encodes a homologous EPSPS or the recombinant polynucleotide encodes a homologous mutant EPSPS.

In one embodiment, the sequence encoding a homologous mutant EPSPS encodes alanine at the amino acid position corresponding to amino acid 96 of the E. coli EPSPS (Genbank Accession No. A7ZYL1; GI: 166988249) (SEQ ID NO: 69). In another embodiment, the sequence encoding a homologous mutant EPSPS encodes threonine at the amino acid position corresponding to amino acid 183 of the E. coli EPSPS (Genbank Accession No. A7ZYL1; GI: 166988249) (SEQ ID NO: 69). In yet another embodiment, the sequence encoding a homologous mutant EPSPS encodes alanine at the amino acid position corresponding to amino acid 96 and threonine at the amino acid position corresponding to amino acid 183, of the E. coli EPSPS (Genbank Accession No. A7ZYL1; GI: 166988249) (SEQ ID NO: 69). In one embodiment, the recombinant polynucleotide encodes a heterologous EPSPS protein.

In another embodiment, the sequence encoding the EPSPS is codon biased to reflect the codon bias of the chloroplast genome of the alga.

In yet another embodiment, the sequence encoding the EPSPS is operably linked to a promoter that functions in the chloroplast of the alga. In some embodiments, the promoter that functions in the chloroplast of the alga comprises a 16SrRNA promoter, an rbcL promoter, an atpA promoter, a psaA promoter, a psbA promoter, or a psbD promoter. In other embodiments, the sequence encoding the EPSPS is operably linked to a 5′ UTR that functions in the chloroplast of the alga or the sequence encoding the EPSPS is operably linked to a 3′ UTR that functions in the chloroplast of the alga. In one embodiment, the recombinant polynucleotide further comprises a transcriptional regulatory sequence for expression of the polynucleotide in the chloroplast of the alga.

In one embodiment, the alga is a non-chlorophyll c-containing eukaryotic alga. In another embodiment, the alga is green alga. In some embodiments, the green alga is a Chlorophycean, Chlamydomonas, Scenedesmus, Chlorella, or Nannochlorpis. In one embodiment, the Chlamydomonas is C. reinhardtii. In another embodiment, the Chlamydomonas is C. reinhardtii 137c. In one embodiment, the alga is a microalga. In some embodiments, the microalga is a Chlamydomonas, Volvacales, Dunaliella, Scenedesmus, Chlorella, or Hematococcus species. In one embodiment, the alga is a macroalga.

Provided herein are glyphosate resistant prokaryotic alga comprising a recombinant polynucleotide integrated into the genome of the alga, wherein the recombinant polynucleotide comprises a sequence encoding a 5-enolpyruvylshikimate-3-phosphate synthase (EPSPS) that confers glyphosate resistance to the alga.

In some embodiments, the recombinant polynucleotide encodes a homologous EPSPS, the recombinant polynucleotide encodes a homologous mutant EPSPS, or the recombinant polynucleotide encodes a heterologous EPSPS protein.

In one embodiment, the sequence encoding the EPSPS is codon biased to reflect the codon bias of the genome of the alga.

In another embodiment, the sequence encoding the EPSPS is operably linked to a promoter. In some embodiments, the promoter comprises a 16SrRNA promoter, an rbcL promoter, an atpA promoter, a psaA promoter, a psbA promoter, or a psbD promoter. In one embodiment, the sequence encoding the EPSPS is operably linked to a 5′ UTR. In yet another embodiment, the sequence encoding the EPSPS is operably linked to a 3′ UTR. In another embodiment, the recombinant polynucleotide further comprises a transcriptional regulatory sequence for expression of the polynucleotide in the alga.

In one embodiment, the prokaryotic alga is a cyanobacteria. In other embodiments, the cyanobacteria can be a Synechococcus, Synechocystis, Athrospira, Anacytis, Anabaena, Nostoc, Spirulina, or Fremyella species.

Also provided herein are glyphosate resistant eukaryotic alga comprising a heterologous polynucleotide integrated into the chloroplast genome, wherein the heterologous polynucleotide comprises a sequence that encodes glyphosate oxidoreductase (GOX), glyphosate acetyl transferase (GAT), or a Class II EPSP synthase.

In some embodiments, the sequence that encodes glyphosate oxidoreductase (GOX), glyphosate acetyl transferase (GAT), or a Class II EPSP synthase, is codon biased to reflect the codon bias of the chloroplast genome of the alga. In other embodiments, the sequence that encodes glyphosate oxidoreductase (GOX), glyphosate acetyl transferase (GAT), or a Class II EPSP synthase, is operably linked to a promoter that functions in the chloroplast of the alga.

In yet other embodiments, the promoter that functions in the chloroplast of the alga is a 16SrRNA promoter, an rbcL promoter, an atpA promoter, a psaA promoter, a psbA promoter, or a psbD promoter. In some embodiments, the sequence that encodes glyphosate oxidoreductase (GOX), glyphosate acetyl transferase (GAT), or a Class II EPSP synthase, is operably linked to a 5′ UTR that functions in the chloroplast of the alga. In other embodiments, the sequence that encodes glyphosate oxidoreductase (GOX), glyphosate acetyl transferase (GAT), or a Class II EPSP synthase, is operably linked to a 3′ UTR that functions in the chloroplast of the alga.

In one embodiment, the alga is green alga. In other embodiments, the green alga is a Chlorophycean. Chlamydomonas, Scenedesmus, Chlorella, or Nannochlorpis. In one embodiments, the Chlamydomonas is C. reinhardtii. In another embodiment, the Chlamydomonas is C. reinhardtii 137c. In yet another embodiment, the alga is a microalga. In some embodiments, the microalga is a Chlamydomonas, Volvacales, Dunaliella, Scenedesmus, Chlorella, or Hematococcus species. In one embodiment, the alga is a macroalga.

In addition, provided herein are non-antibiotic herbicide resistant eukaryotic alga comprising a polynucleotide integrated into the chloroplast genome, wherein the polynucleotide comprises a sequence encoding a heterologous protein whose wild-type form is not encoded by the chloroplast genome, wherein the protein confers resistance to a non-antibiotic herbicide that does not inhibit amino acid synthesis.

In some embodiments, the non-antibiotic herbicide is a 1,2,4-triazol pyrimidine, aminotriazole amitrole, an isoxazolidinone, an isoxazole, a diketonitrile, a triketone, a pyrazolinate, norflurazon, a bipyridylium, an aryloxyphenoxy propionate, a cyclohexandione oxime, a p-nitrodiphenylether, an oxadiazole, an N-phenyl imide, a halogenated hydrobenzonitrile, or a urea herbicide.

In other embodiments, the sequence encoding the heterologous protein encodes glutathione reductase, superoxide dismutase (SOD), acetohydroxy acid synthase (AHAS), bromoxynil nitrilase, hydroxyphenylpyruvate dioxygenase (HPPD), isoprenyl pyrophosphate isomerase, prenyl transferase, lycopene cyclase, phytoene desaturase, acetyl CoA carboxylase (ACCase), or cytochrome P450-NADH-cytochrome P450 oxidoreductase.

In one embodiment, the sequence encoding the heterologous protein is codon biased to reflect the codon bias of the chloroplast genome of the alga.

In another embodiment, the sequence encoding the heterologous protein is operably linked to a promoter that functions in the chloroplast of the alga. In yet other embodiments, the promoter that functions in the chloroplast of the alga is a 16SrRNA promoter, an rbcL promoter, an atpA promoter, a psaA promoter, a psbA promoter, or a psbD promoter. In one embodiment, the sequence encoding the heterologous protein is operably linked to a 5′ UTR that functions in the chloroplast of the alga. In another embodiment, the sequence encoding the heterologous protein is operably linked to a 3′ UTR that functions in the chloroplast of the alga.

In yet another embodiment, the alga is green alga. In some embodiments, the green alga is a Chlorophycean, Chlamydomonas, Scenedesmus, Chlorella, or Nannochlorpis. In one embodiment, the Chlamydomonas is C. reinhardtii. In another embodiment, the Chlamydomonas is C. reinhardtii 137c. In yet another embodiment, the alga is a microalga. In other embodiments, the microalga is a Chlamydomonas, Volvacales, Dunaliella, Scenedesmus, Chlorella, or Hematococcus species. In one embodiment, the alga is a macroalga.

Also provided herein are glyphosate resistant non-chlorophyll c-containing eukaryotic alga comprising a heterologous polynucleotide integrated into the nuclear genome, wherein the heterologous polynucleotide comprises a sequence that encodes a protein that confers resistance to glyphosate.

In some embodiments, the protein is 5-enolpyruvylshikimate-3-phosphate synthase (EPSPS), glyphosate oxidoreductase (GOX), or glyphosate acetyl transferase (GAT).

In one embodiment, the protein is 5-enolpyruvylshikimate-3-phosphate synthase (EPSPS). In other embodiments, the protein is a homologous EPSPS, the protein is a homologous mutant EPSPS, or the protein is a heterologous EPSPS.

In one embodiment, the sequence that encodes the protein is codon biased to reflect the codon bias of the nuclear genome of the alga.

In another embodiment, the sequence that encodes the protein is operably linked to a promoter that functions in the nucleus of the alga. In some embodiments, the promoter that functions in the nucleus of the alga is a rbcS promoter, an LHCP promoter, or a nitrate reductase promoter. In other embodiments, the sequence that encodes the protein is operably linked to a 5′ UTR that functions in the nucleus of the alga, or the sequence that encodes the protein is operably linked to a 3′ UTR that functions in the nucleus of the alga.

In one embodiment, the alga is green alga. In other embodiments, the green alga is a Chlorophycean, Chlamydomonas, Scenedesmus, Chlorella, or Nannochlorpis. In one embodiment, the Chlamydomonas is C. reinhardtii. In another embodiment, the Chlamydomonas is C. reinhardtii 137c. In yet another embodiment, the alga is a microalga. In some embodiments, the microalga is a Chlamydomonas, Volvacales, Dunaliella, Scenedesmus, Chlorella, or Hematococcus species. In one embodiment, the alga is a macroalga.

Provided herein are herbicide resistant non-chlorophyll c-containing eukaryotic alga comprising a heterologous polynucleotide integrated into the nuclear genome, wherein the heterologous polynucleotide comprises a sequence that encodes a protein that confers herbicide resistance to the alga.

In one embodiment, the sequence that encodes the protein is codon biased to reflect the codon bias of the nuclear genome of the alga.

In another embodiment, the sequence that encodes the protein is operably linked to a heterologous promoter. In some embodiments, the sequence that encodes the protein is operably linked to a 5′ UTR that functions in the nucleus of the alga, or the sequence that encodes the protein is operably linked to a 3′ UTR that functions in the nucleus of the alga.

In one embodiment, the heterologous polynucleotide further comprises genomic sequences flanking the sequence that encodes the protein, wherein the genomic sequences are homologous to sequences of the genome of the non-chlorophyll c-containing eukaryotic alga.

In other embodiments, the protein is 5-enolpyruvylshikimate-3-phosphate synthase (EPSPS), glyphosate oxidoreductase (GOX), glyphosate acetyl transferase (GAT), phosphinothricin acetyl transferase (PAT), glutathione reductase, superoxide dismutase (SOD), acetolactate synthase (ALS), acetohydroxy acid synthase (AHAS), hydroxyphenylpyruvate dioxygenase (HPPD), bromoxynil nitrilase, hydroxyphenylpyruvate dioxygenase (HPPD), isoprenyl pyrophosphate isomerase, prenyl transferase, lycopene cyclase, phytoene desaturase, acetyl CoA carboxylase (ACCase), or cytochrome P450-NADH-cytochrome P450 oxidoreductase.

In one embodiment, the protein confers resistance to a non-antibiotic herbicide. In another embodiment, the protein confers resistance to glyphosate. In other embodiments, the protein is 5-enolpyruvylshikimate-3-phosphate synthase (EPSPS), glyphosate oxidoreductase (GOX), or glyphosate acetyl transferase (GAT). In one embodiment, the protein is 5-enolpyruvylshikimate-3-phosphate synthase (EPSPS).

In one embodiment, the alga is green alga. In other embodiments, the green alga is a Chlorophycean, Chlamydomonas, Scenedesmus, Chlorella, or Nannochlorpis. In one embodiment, the Chlamydomonas is C. reinhardtii. In another embodiment, the Chlamydomonas is C. reinhardtii 137c. In yet another embodiment, the alga is a microalga. In some embodiments, the microalga is a Chlamydomonas, Volvacales, Dunaliella, Scenedesmus, Chlorella, or Hematococcus species. In one embodiment, the alga is a macroalga.

Also provided herein are herbicide resistant eukaryotic alga comprising two or more polynucleotide sequences encoding proteins that confer resistance to herbicides, wherein each of the proteins confers resistance to a different herbicide.

In some embodiments, the polynucleotide sequence is a homologous polynucleotide sequence, the polynucleotide sequences is a homologous mutant polynucleotide sequence, or the polynucleotide sequences is a heterologous polynucleotide sequence.

In another embodiment, at least one of the polynucleotide sequences is incorporated into the chloroplast genome of the alga. In yet another embodiment, the polynucleotide sequence that is incorporated into the chloroplast genome comprises a protein encoding sequence that is codon biased to reflect the codon bias of the chloroplast genome of the alga.

In one embodiment, at least one of the polynucleotides is incorporated into the nuclear genome of the alga. In yet another embodiment, the polynucleotide sequence that is incorporated into the nuclear genome comprises a protein encoding sequence that is codon biased to reflect the codon bias of the nuclear genome of the alga.

In another embodiment, at least one of the polynucleotides is incorporated into the chloroplast genome of the alga and at least one of the polynucleotides is incorporated into the nuclear genome of the alga.

In one embodiment, the alga is green alga. In other embodiments, the green alga is a Chlorophycean, Chlamydomonas, Scenedesmus, Chlorella, or Nannochlorpis. In yet another embodiment, the Chlamydomonas is C. reinhardtii. In one embodiment, the Chlamydomonas is C. reinhardtii 137c. In yet another embodiment, the alga is a microalga. In some embodiments, the microalga is a Chlamydomonas, Volvacales, Dunaliella, Scenedesmus, Chlorella, or Hematococcus species. In one embodiment, the alga is a macroalga.

In addition, provided herein are non chlorophyll c-containing herbicide resistant alga comprising a polynucleotide encoding a protein that confers resistance to a herbicide and a heterologous polynucleotide encoding a protein that does not confer resistance to a herbicide, wherein the protein that does not confer resistance to a herbicide is an industrial enzyme, a protein that participates in or promotes the synthesis of at least one nutritional, therapeutic, commercial, or fuel biomolecule, or a protein that facilitates the isolation of at least one nutritional, therapeutic, commercial, or fuel biomolecule.

In one embodiment, the protein that does not confer resistance to a herbicide is an industrial enzyme. In one aspect, the protein that does not confer resistance to a herbicide is a protein that participates in or promotes the synthesis of at least one nutritional, therapeutic, commercial, or fuel biomolecule, or is a protein that facilitates the isolation of at least one nutritional, therapeutic, commercial, or fuel biomolecule. In other embodiments, the nutritional biomolecule comprises a lipid, a carotenoid, a fatty acid, a vitamin, a cofactor, a nucleotide, an amino acid, a peptide, or a protein. In some embodiments, the therapeutic biomolecule comprises a vitamin, a cofactor, an amino acid, a peptide, a hormone, or a growth factor. In other embodiments, the commercial biomolecule comprises a lubricant, a perfume, a pigment, a coloring agent, a flavoring agent, an enzyme, an adhesive, a thickener, a solubilizer, a stabilizer, a surfactant, or a coating. In still other embodiments, the fuel biomolecule comprises a lipid, a fatty acid, a hydrocarbon, a carbohydrate, cellulose, glycerol, or an alcohol.

In one embodiment, the polynucleotide encoding a protein that confers resistance to a herbicide is a heterologous polynucleotide. In another embodiment, the polynucleotide encoding a protein that confers resistance to a herbicide is a homologous polynucleotide. In one embodiment, the polynucleotide encoding a protein that confers resistance to a herbicide is a homologous mutant polynucleotide.

In another embodiment, the alga is a microalga. In yet embodiment, the alga is a cyanobacterium. In other embodiments, the alga is a Synechococcus, Anacytis, Anabaena, Athrospira, Nostoc, Spirulina, or Fremyella species. In one embodiment, the alga is a eukaryotic alga. In yet other embodiments, the alga is a Chlamydomonas, Volvacales, Dunaliella, Scenedesmus, Chlorella, or Hematococcus species. In one embodiment, Chlamydomonas is C. reinhardtii. In yet another embodiment, the Chlamydomonas is C. reinhardtii 137c. In another embodiment, the alga is a macroalga.

In one embodiment, the polynucleotide encoding a protein that confers resistance to a herbicide is integrated into the nuclear genome. In another embodiment, the polynucleotide encoding a protein that confers resistance to a herbicide is integrated into the chloroplast genome. In yet another embodiment, the heterologous polynucleotide encoding a protein that does not confer resistance to a herbicide is integrated into the nuclear genome. In another embodiment, the heterologous polynucleotide encoding a protein that does not confer resistance to a herbicide is integrated into the chloroplast genome.

In another aspect, the non chlorophyll c-containing herbicide resistant alga comprise two or more polynucleotides encoding proteins that confer resistance to herbicides, wherein each of the proteins confers resistance to a different herbicide. In one embodiment, at least one of the two or more polynucleotides is integrated into the chloroplast genome. In another embodiment, at least one of the two or more polynucleotides is integrated into the nuclear genome.

In another aspect, the non chlorophyll c-containing herbicide resistant alga comprise two or more heterologous polynucleotides encoding proteins that do not confer resistance to a herbicide, wherein each of the two or more proteins that do not confer herbicide resistance is a protein that participates in or promotes the synthesis of at least one nutritional, therapeutic, commercial, or fuel biomolecule, or is a protein that facilitates the isolation of at least one nutritional, therapeutic, commercial, or fuel biomolecule. In one embodiment, at least one of the two or more heterologous polynucleotides are integrated into the chloroplast genome. In another embodiment, at least one of the two or more heterologous polynucleotides are integrated into the nuclear genome.

In yet another embodiment, the heterologous polynucleotide(s) integrated into the nuclear genome is (are) operably linked to a regulatable promoter. In another embodiment, the regulatable promoter can be induced or repressed by one or more compounds added to the growth media of the alga.

In yet another embodiment, one or more compounds is nitrate, sulfate, an amino acid, a vitamin, a sugar, a nucleotide or nucleoside, an antibiotic, or a hormone.

Also provided herein are methods for producing one or more biomolecules, comprising: (a) transforming an alga with a polynucleotide comprising a sequence conferring herbicide resistant to the alga; (b) growing the alga in the presence of the herbicide; and (c) harvesting one or more biomolecules from the alga.

In one embodiment, the herbicide resistant alga is used to inoculate media or a body of water that includes at least one herbicide. In another embodiment, the herbicide is a non-antibiotic herbicide. In some embodiments, the herbicide is glyphosate, a sulfonylurea, an imidazolinone, a 1,2,4-triazol pyrimidine, phosphinothricin, aminotriazole amitrole, an isoxazolidinones, an isoxazole, a diketonitrile, a triketone, a pyrazolinate, norflurazon, a bipyridylium, a p-nitrodiphenylether, an oxadiazole, an aryloxyphenoxy propionate, a cyclohexandione oxime, a triazine, diuron, DCMU, chlorsulfuron, imazaquin, an N-phenyl imide, a phenol herbicide, a halogenated hydrobenzonitrile, or a urea herbicide. In one embodiment, the herbicide is glyphosate.

In yet another embodiment, the sequence conferring herbicide resistance encodes 5-enolpyruvylshikimate-3-phosphate synthase (EPSPS).

In other embodiments, the methods further comprise transforming the alga with an additional polynucleotide comprising a sequence conferring resistance to a different herbicide, wherein growing the alga in the presence of the herbicide comprises growing the alga in the presence of the herbicide and the different herbicide. In one embodiment, growing the alga in the presence of the herbicide is growing the alga in a liquid medium that comprises at least one nutrient and at least one herbicide. In another embodiment, the alga is grown in an open pond.

In some embodiments, at least one of the one or more biomolecules is a therapeutic protein or an industrial enzyme. In one embodiment, at least one biomolecule is a fuel biomolecule.

In some embodiments, the methods further comprise transforming the alga with a polynucleotide encoding a therapeutic protein or an industrial enzyme. In other embodiments, the methods further comprise transforming the alga with a polynucleotide that increases production of at least one fuel biomolecule. In some embodiments, the methods further comprise transforming the alga with a polynucleotide encoding a flocculation moiety or with a polynucleotide that promotes increased expression of a naturally occurring flocculation moiety or dewatering the alga by flocculating the alga.

In one embodiment, the alga is a eukaryotic alga.

In another embodiment, the polynucleotide comprises a sequence conferring herbicide tolerance is transformed into the algal chloroplast genome.

In yet another embodiment, the alga is a cyanobacterium.

In some embodiments, the methods further comprise providing carbon to the alga.

In some embodiments, the carbon is CO2, flue gas, or acetate.

In some embodiments, the methods further comprise removing nitrogen from chlorophyll of the alga.

Also provided herein are business methods comprising growing recombinant alga resistant to a herbicide in the presence of the herbicide and selling carbon credits resulting from carbon used by the alga.

In one embodiment, the herbicide is glyphosate.

In another embodiment, the alga is green alga. In some embodiments, the green alga is a Chlorophycean, Chlamydomonas, Scenedesmus, Chlorella, or Nannochlorpis. In yet another embodiment, the Chlamydomonas is C. reinhardtii. In one embodiment, the Chlamydomonas is C. reinhardtii 137c. In another embodiment, the alga is a microalga.

In some embodiments, the microalga is a Chlamydomonas, Volvacales, Dunaliella, Scenedesmus, Chlorella, or Hematococcus species. In one embodiment, the alga is a macroalga.

In addition, provided herein are methods of producing a biomass-degrading enzyme in an alga, comprising: (a) transforming the alga with a polynucleotide comprising a sequence conferring herbicide tolerance to the alga and a sequence encoding an exogenous biomass-degrading enzyme or which promotes increased expression of an endogenous biomass-degrading enzyme; and (b) growing the alga in the presence of the herbicide, wherein the herbicide is in sufficient concentration to inhibit growth of the alga which does not comprise the sequence conferring herbicide tolerance, and under conditions which allow for production of the biomass-degrading enzyme, thereby producing the biomass-degrading enzyme.

In one embodiment, the herbicide is glyphosate.

In another embodiment, the biomass-degrading enzyme is chlorophyllase.

Also provided herein are eukaryotic alga comprising a polynucleotide that comprises a sequence encoding Bt toxin integrated into the chloroplast genome. In one embodiment, the polynucleotide that comprises a sequence encoding Bt toxin is a cry gene. In another embodiment, the sequence encoding Bt toxin is codon biased to reflect the codon bias of the chloroplast genome of the alga.

In yet another embodiment, the sequence encoding Bt toxin is operably linked to a promoter that functions in the chloroplast of the alga. In some embodiments, the promoter that functions in the chloroplast of the alga is a 16SrRNA promoter, an rbcL promoter, an atpA promoter, a psaA promoter, a psbA promoter, or a psbD promoter. In another embodiment, the sequence encoding Bt toxin is operably linked to a 5′ UTR that functions in the chloroplast of the alga. In yet another embodiment, the sequence encoding Bt toxin is operably linked to a 3′ UTR that functions in the chloroplast of the alga.

In some embodiments, the alga is a Chlamydomonas, Volvacales, Dunaliella, Scenedesmus, Chlorella, or Hematococcus species.

In one embodiment, the eukaryotic alga further comprise a polynucleotide that encodes a protein that confers resistance to a herbicide. In another embodiment, the polynucleotide that encodes a protein that confers resistance to a herbicide is a heterologous protein. In yet another embodiment, the polynucleotide that encodes a protein that confers resistance to a herbicide is a mutant homologous protein.

Provided herein are eukaryotic alga comprising a polynucleotide that comprises a sequence encoding Bt toxin integrated into the nuclear genome.

In one embodiment, the polynucleotide further comprises a transcriptional regulatory sequence for expression in the nucleus of the alga.

In another embodiment, the alga is a microalga. In some embodiments, the alga is a Chlamydomonas, Volvacales, Dunaliella, Scenedesmus, Chlorella, or Hematococcus species. In yet another embodiment, the alga is a Chlamydomonas species.

In one embodiment, the sequence encoding Bt toxin is codon biased to reflect the codon bias of the nuclear genome of the alga.

In another embodiment, the sequence encoding Bt toxin is operably linked to a promoter that functions in the nucleus of the alga. In some embodiments, the promoter that functions in the nucleus of the alga is a rbcS promoter, an LHCP promoter, or a nitrate reductase promoter.

In one embodiment, the eukaryotic alga further comprises a polynucleotide that encodes a protein that confers resistance to a herbicide.

Also provided herein are prokaryotic alga comprising a polynucleotide that comprises a heterologous sequence encoding Bt toxin.

In one embodiment, the alga is a cyanobacterium. In other embodiments, the alga is a Synechococcus, Anacytis, Anabaena. Athrospira, Nostoc, Spirulina, or Fremyella species.

In yet another embodiment, the sequence encoding Bt toxin is codon biased to reflect the codon bias of the genome of the alga.

In one embodiment, the prokaryotic alga further comprises a polynucleotide that encodes a protein that confers resistance to a herbicide.

In addition, provided herein are isolated polynucleotides for transformation of a non-chlorophyll c-containing alga to herbicide resistance, wherein the polynucleotide comprises a sequence encoding a heterologous protein that confers resistance to a herbicide, wherein the protein encoding sequence is codon biased to reflect the codon bias of the nuclear genome of the alga.

In one embodiment, the protein encoding sequence is codon biased to reflect the codon bias of the nuclear genome of Chlamydomonas reinhardtii.

In another embodiment, the polynucleotide further comprises a promoter active in the nuclear genome of the alga. In some embodiments, the promoter comprises a rbcS promoter, an LHCP promoter, or a nitrate reductase promoter. In yet another embodiment, the polynucleotide further comprises a promoter for expression in the nucleus of Chlamydomonas reinhardtii. In one embodiment, the polynucleotide further comprises a chloroplast transit peptide-encoding sequence.

Presented herein are algae that are genetically engineered for herbicide resistance. A herbicide resistant alga as disclosed herein is transformed with one or more polynucleotides that encode one or more proteins that confer herbicide resistance. Algae that include one or more recombinant nucleic acid molecules encoding one or more herbicide resistance-conferring proteins can be grown in the presence of one or more herbicides that can deter the growth of other algae and, in some embodiments, other non-algal organisms. Also provided are algae transformed with a polynucleotide that encodes a protein that is toxic to one or more animal species, such as a gene encoding a Bt toxin that is lethal to insects.

Algae transformed with one or more polynucleotides that include one or more herbicide resistance genes are in some embodiments grown on a large scale in the presence of herbicide for the production of biomolecules, such as, for example, therapeutic proteins, industrial enzymes, nutritional molecules, commercial products, or fuel products. Algae transformed with one or more toxin genes that are lethal to one or more insect species can also be grown in large scale for production of therapeutic, nutritional, fuel, or commercial products. Algae bioengineered for herbicide resistance and/or to express insect toxins can also be grown in large scale cultures for decontamination of compounds, environmental remediation, or carbon fixation.

A herbicide resistance gene used to transform algae can confer resistance to any type of herbicide, including but not limited to herbicides that inhibit amino acid biosynthesis, herbicides that inhibit photosynthesis, herbicides that inhibit carotenoid biosynthesis, herbicides that inhibit fatty acid biosynthesis, photobleaching herbicides, etc.

Provided in some embodiments herein is a herbicide resistant prokaryotic alga transformed with a recombinant polynucleotide encoding a protein that confers herbicide resistance. In some embodiments, the alga is a cyanobacteria species. A recombinant polynucleotide encoding a herbicide resistance gene is in some embodiments integrated into the genome of a prokaryotic host alga.

In some embodiments, the host alga transformed with a herbicide resistance gene is a eukaryotic alga. In some embodiments, the host alga is a species of the Chlorophyta. In some embodiments, the alga is a microalga. In some instances, the microalga is a Chlamydomonas species. A recombinant polynucleotide conferring herbicide resistance can be integrated into the nuclear genome or chloroplast genome of a eukaryotic host alga. A transformed alga having a herbicide resistance gene incorporated into the chloroplast genome is in some embodiments homoplastic for the herbicide resistance gene.

In one instance, provided herein is a glyphosate resistant eukaryotic alga, in which the eukaryotic alga contains a polynucleotide encoding a homologous mutant 5-enolpyruvylshikimate-3-phosphate synthase (EPSPS) integrated into the chloroplast genome, in which the homologous mutant EPSP synthase confers glyphosate resistance.

In another instance, provided herein is a herbicide resistant eukaryotic microalga containing a heterologous polynucleotide integrated into the chloroplast genome, in which the heterologous polynucleotide comprises a sequence that encodes a glyphosate oxidoreductase (GOX), a glyphosate acetyl transferase (GAT), or an EPSP synthase that is not a Class I EPSP synthase.

In a further instance, a herbicide resistant eukaryotic alga comprises a heterologous polynucleotide integrated into the chloroplast genome, in which the heterologous polynucleotide encodes a protein whose wild-type form is not encoded by the chloroplast genome, in which the protein confers resistance to a non-antibiotic herbicide that does not inhibit amino acid synthesis.

In another embodiment, provided herein is a herbicide-resistant non-chlorophyll c-containing eukaryotic alga comprising a heterologous polynucleotide integrated into the nuclear genome, wherein the heterologous polynucleotide comprises a sequence that encodes a protein that confers resistance to a herbicide, wherein resistance to the herbicide is conferred by a single heterologous protein.

In another embodiment, provided herein is a herbicide resistant non-chlorophyll c-containing eukaryotic alga comprising a heterologous polynucleotide integrated into the nuclear genome, wherein the heterologous polynucleotide comprises a sequence that encodes a protein that confers resistance to glyphosate.

Also provided herein is a herbicide-resistant non-chlorophyll c-containing eukaryotic alga comprising a recombinant polynucleotide integrated into the nuclear genome, in which the recombinant polynucleotide encodes a homologous EPSPS protein that confers resistance to glyphosate.

Also provided are nucleic acid constructs for transforming algae with one or more nucleotide sequences that confer herbicide resistance. The disclosure includes recombinant polynucleotides containing a sequence that encodes a protein that confers resistance to a herbicide, in which the herbicide resistance gene sequence is operably linked to one or more of 1) a transcriptional regulatory sequence that is functional in the chloroplast genome of a host alga, 2) a transcriptional regulatory sequence that is functional in the nuclear genome of a host alga, 3) a translational regulatory sequence that is functional in the chloroplast genome of a host alga, 4) a translational regulatory sequence that is functional in the nuclear genome of a host alga, 5) one or more sequences having homology to the chloroplast genome of the host alga, and 6) one or more sequences having homology to the nuclear genome of the host alga. The sequence that encodes a protein that encodes resistance to a herbicide can be a homologous or heterologous sequence with respect to the host alga, and can optionally include one or more mutations with respect to the sequence from which it is derived.

In some instances, the nucleic acid sequence that encodes a protein that confers herbicide resistance is codon-biased. The nucleic acid sequence that encodes a protein that confers herbicide resistance in some embodiments is codon-biased to conform to the codon bias of the genome of a prokaryotic host alga. The nucleic acid sequence that encodes a protein that confers herbicide resistance in some embodiments is codon-biased to conform to the codon usage bias of the chloroplast genome of a eukaryotic host alga. The nucleic acid sequence that encodes a protein that confers herbicide resistance in some embodiments is codon-biased to conform to the codon usage bias of the nuclear genome of a eukaryotic host alga. Disclosed in one aspect is an isolated polynucleotide for transformation of a non-chlorophyll c-containing alga to herbicide resistance, wherein the polynucleotide comprises a sequence encoding a heterologous protein that confers resistance to a herbicide, wherein the protein-encoding sequence is codon biased for the nuclear genome of the alga.

The disclosure further provides an alga comprising a recombinant polynucleotide that encodes a Bacillus thuringiensis (Bt) toxin protein. In one embodiment, the alga includes a cry gene encoding the Bt toxin. The heterologous Bt toxin gene can be incorporated in to the nuclear genome or the chloroplast genome of the alga. The alga having a heterologous Bt toxin gene can further include one or more recombinant nucleotides that encode a protein conferring resistance to a herbicide.

The disclosure further provides a herbicide-resistant eukaryotic alga comprising two or more recombinant polynucleotide sequences encoding proteins that confer resistance to herbicides, in which each of the proteins confers resistance to a different herbicide. In one embodiment, at least one of the polynucleotide sequences encoding a protein conferring herbicide resistance is integrated into the chloroplast genome of a eukaryotic alga. In one embodiment, at least one of the polynucleotide sequences encoding a protein conferring herbicide resistance is integrated into the nuclear genome of a eukaryotic alga. In a further embodiment, at least a first of the two or more polynucleotide sequences encoding a protein conferring herbicide resistance is integrated into the chloroplast genome and at least a second of the two or more polynucleotide sequences encoding a protein conferring herbicide resistance is integrated into the nuclear genome of a eukaryotic alga.

Also provided herein is a non chlorophyll c-containing herbicide-resistant alga comprising a polynucleotide encoding a protein that confers resistance to a herbicide and a heterologous polynucleotide encoding a protein that does not confer resistance to a herbicide, wherein the protein that does not confer resistance to a herbicide is an industrial enzyme or therapeutic protein, or a protein that participates in or promotes the synthesis of at least one nutritional, therapeutic, commercial, or fuel product, or a protein that facilitates the isolation of at least one nutritional, therapeutic, commercial, or fuel product.

Also disclosed herein are methods of producing one or more biomolecules, in which the methods include transforming an alga with a polynucleotide comprising a sequence conferring herbicide tolerance, growing the alga in the presence of the herbicide, and harvesting one or more biomolecules from the alga or algal media. The methods in some embodiments include isolating the one or more biomolecules.

Further included are methods of producing one or more biomolecules, in which the methods include transforming an alga with a polynucleotide comprising a sequence encoding a toxin that impedes the growth of at least one animal species, growing the alga under conditions in which the toxin is expressed, and harvesting one or more biomolecules from the alga or algal media. The methods in some embodiments include isolating the one or more biomolecules.

In some embodiments, algae are transformed with at least one herbicide resistance gene and at least one toxin gene, and are grown in the presence of at least one herbicide under conditions in which the toxin is expressed, and one or more biomolecules is harvested from the alga or algal media.

Also disclosed herein are methods of producing a biomass-degrading enzyme in an alga, in which the methods include transforming the alga with a polynucleotide comprising a sequence conferring herbicide tolerance to the alga and a sequence encoding an exogenous biomass-degrading enzyme or which promotes increased expression of an endogenous biomass-degrading enzyme; growing the alga in the presence of the herbicide and under conditions which allow for production of the biomass-degrading enzyme, in which the herbicide is in sufficient concentration to inhibit growth of the alga which does not include the sequence conferring herbicide tolerance, to producing the biomass-degrading enzyme. The methods in some embodiments include isolating the biomass-degrading enzyme.

### BRIEF DESCRIPTION OF THE DRAWINGS

These and other features, aspects, and advantages of the present disclosure will become better understood with regard to the following description, appended claims and accompanying figures where:

FIG. 1 provides schematic diagrams of exemplary nucleic acid constructs that can be used to transform algae.

FIG. 2 provides schematic diagrams of exemplary nucleic acid constructs that can be used to transform algae.

FIG. 3 provides schematic diagrams of exemplary nucleic acid constructs that can be used to transform algae.

FIG. 4 shows a western blot of C. reinhardtii strains engineered with C. reinhardtii EPSPS cDNA mutated at G163A and A252T in the chloroplast genome to confer glyphosate resistance. This western blot shows the expression of the double mutant driven by both the psbD and atpA promoters.

FIG. 5 shows glyphosate resistance of C. reinhardtii strains engineered with C. reinhardtii EPSPS cDNA mutated at G163A and A252T driven by the psbD and atpA promoters in the chloroplast genome as compared with C. reinhardtii WT cc1690. The engineered strains show enhanced glyphosate resistance.

FIG. 6 shows a western blot of the expression of C. reinhardtii EPSPS cDNA in Escherichia coli (1) and the mutant forms G163A, A252T, and G163A/A252T of C. reinhardtii EPSPS cDNA from the C. reinhardtii nuclear genome (2,3, and 4, respectively). Expression of the C. reinhardtii EPSPS cDNA in E. coli results in the chloroplast targeting peptide (CTP) remaining intact. However, expression of EPSPS cDNA in C. reinhardtii results in both protein bands (+CTP and −CTP) indicating the presence of the targeting activity.

FIG. 7 shows strains engineered in the nuclear genome with C. reinhardtii EPSPS cDNA mutated at G163A, A252T, and G163A/A252T to confer glyphosate resistance. The box represents an unengineered C. reinhardtii WT cc1690 negative control. These strains are plated on 2 mM glyphosate. The circles indicate engineered strains with particularly higher glyphosate resistance due to the positional effect.

FIG. 8 shows strains engineered in the nuclear genome with C. reinhardtii EPSPS nuclear wild type DNA (introns and exons), mutated at G163A, A252T, and G163A/A252T to confer glyphosate resistance. The box represents an unengineered C. reinhardtii WT cc1690 negative control. These strains are plated on 4 mM glyphosate. The circle indicates the strain that was taken for liquid culture characterization in FIG. 9. The frequency of highly resistant strains in the double mutant are indicative of the combined effects of the mutation.

FIG. 9 shows further characterization of glyphosate resistance in an engineered C. reinhardtii strain overexpressing another copy of C. reinhardtii EPSPS nuclear DNA (introns and exons); high resistance to glyphosate is shown. C. reinhardtii WT cc1690 is included in the first row as a negative control.

FIG. 10 provides a schematic diagram of an exemplary nucleic acid construct that can be used to transform algae.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this specification and the appended claims, the singular forms “a”, “an” and “the” include plural reference unless the context clearly dictates otherwise.

### Algae

The present disclosure provides algae and algal cells transformed with one or more polynucleotides that confer herbicide resistance. Also provided are algae and algal cells transformed with a polynucleotide encoding the Bt toxin that is lethal to some insect and rotifer species. The transformed algae may be referred to herein as “host algae”.

Algae transformed with herbicide resistance genes or a gene encoding Bt toxin as disclosed herein can be macroalgae or microalgae. Microalgae include eukaryotic microalgae and cyanobacteria. In some embodiments, herbicide resistant algae are provided that comprise a polynucleotide encoding a protein that confers resistance to a herbicide. In some embodiments, the alga is a prokaryotic alga. Examples of some prokaryotic alga of the present disclosure include, but are not limited to cyanobacteria. Examples of cyanobacteria include, for example, Synechococcus, Synechocystis, Athrospira, Anacytis, Anabaena, Nostoc, Spirulina, and Fremyella species.

In some embodiments, the alga is eukaryotic. The alga can be unicellular or multicellular. Examples of algae contemplated herein include, but are not limited to, members of the order rhodophyta (red algae), chlorophyta (green algae), phaeophyta (brown algae), chrysophyta (diatoms and golden brown algae), pyrrophyta (dinoflagellates), and euglenophyta (euglenoids). Other examples of alga are members of the order heterokontophyta, tribophyta, glaucophyta, chlorarachniophytes, haptophyta, cryptomonads, and phytoplankton. In some embodiments, the alga is not a diatom. In some embodiments, the alga is not a brown alga. In some embodiments, the alga is not a chlorophyll c-containing alga.

An exemplary group of algae contemplated for use herein are species of the green algae (Chlorophyta). In some embodiments, eukaryotic microalgae, such as for example, a Chlamydomonas, Volvacales, Dunaliella, Scenedesmus, Chlorella, or Hematococcus species, are used in the disclosed methods. One example, Chlamydomonas, is a genus of unicellular green algae. These algae are found in soil, fresh water, oceans, and even in snow on mountaintops. Algae in this genus have a cell wall, a chloroplast, and two anterior flagella allowing mobility in liquid environments. More than 500 different species of Chlamydomonas have been described.

A commonly used laboratory species is C. reinhardtii. Cells of this species are haploid, and can grow on a simple medium of inorganic salts, using photosynthesis to provide energy. They can also grow in total darkness if acetate is provided as a carbon source. When deprived of nitrogen, C. reinhardtii cells can differentiate into isogametes. Two distinct mating types, designated mt+ and mt−, exist. These fuse sexually, thereby generating a thick-walled zygote which forms a hard outer wall that protects it from various environmental conditions. When restored to nitrogen culture medium in the presence of light and water, the diploid zygospore undergoes meiosis and releases four haploid cells that resume the vegetative life cycle. In mitotic growth the cells double as fast as every eight hours. C. reinhardtii cells can grow under a wide array of conditions. While a dedicated, temperature-controlled space can result in optimal growth, C. reinhardtii can be readily grown at room temperature under standard fluorescent lights. The cells can be synchronized by placing them on a light-dark cycle.

The nuclear genetics of C. reinhardtii is well established. There are a large number of mutant strains that have been characterized and the C. reinhardtii center (www.chlamy.org; Chlamydomonas Center, Duke University) maintains an extensive collection of mutants, as well as annotated genomic sequences of Chlamydomonas species. A large number of chloroplast mutants as well as several mitochondrial mutants have been developed in C. reinhardtii.

An exemplary group of algae contemplated for use herein are green alga. The green alga can be, for example, a Chlorophycean, Chlamydomonas, Scenedesmus, Chlorella, or Nannochlorpis species. The algae can be, for example, Chlamydomonas, specifically, C. reinhardtii. The algae can also be, for example, C. reinhardtii 137c.

Algae, including cyanobacteria, such as, but not limited to Synechococcus, Synechocystis. Athrospira, Anacytis, Anabaena, Nostoc, Spirulina, and Fremyella species, and including green microalgae, such as, but not limited to Dunaliella, Scenedesmus, Chlorella, Volvox, or Hematococcus species can be used in the methods disclosed herein.

### Mutations/Other Mutant Strains

Other exemplary mutations that can be made and used in the disclosed embodiments are provided below.

Mutations can be made to the nucleic acid sequence of a gene, for example, the nucleic acid sequence of the acetolactate synthase large subunit gene. The amino acid sequence of the wild type acetolactate synthase large subunit gene is shown in SEQ ID NO:61. The mutations can be, for example, homologous mutations based on the corresponding amino acid sequence contained in other organisms, for example, Arabidopsis thaliana, that confer resistance to herbicides, for example, chlorsulfuron, and imazaquin. Possible mutations that can be made to the nucleic acid that corresponds to SEQ ID NO:61 are: P198S, R199S, A206V, D377E, W580L, and G666I. Any one or more mutations can be made to the nucleic acid that corresponds to SEQ ID NO: 61.

Mutations can be made to the nucleic acid sequence of a gene, for example, the nucleic acid sequence of the EPSPS gene. The amino acid sequence of the wild type EPSPS gene is shown in SEQ ID NO: 1. The mutations can be, for example, homologous mutations based on the corresponding amino acid sequence contained in other organisms, for example, E. coli, that confer resistance to herbicides, for example, glyphosate. Possible mutations that can be made to the nucleic acid that corresponds to SEQ ID NO:1 are G163A, A252T, K110M, P168S, and T164I/P168S. Any one or more mutations can be made to the nucleic acid that corresponds to SEQ ID NO: 1.

### Transformation of Algal Cells

Transformed cells are produced by introducing homologous and/or heterologous DNA into a population of target cells and selecting the cells which have taken up the DNA. For example, transformants containing exogenous DNA with a selectable marker which confers resistance to kanamycin may be grown in an environment containing kanamycin. Exemplary concentrations of kanamycin that can be used are 50 to 200 μg/ml, or 100 μg/ml. In some embodiments, transformants containing exogenous DNA encoding a protein that confers resistance to a herbicide may be grown in the presence of the herbicide to select for transformants. The polynucleotide conferring herbicide resistance can be introduced into an algal cell using a direct gene transfer method such as, for example, electroporation, microprojectile mediated (biolistic) transformation using a particle gun, the “glass bead method,” or by cationic lipid or liposome-mediated transformation.

The basic techniques used for transformation and expression in photosynthetic organisms are similar to those commonly used for E. coli, Saccharomyces cerevisiae, and other species. Transformation methods customized for cyanobacteria, or the chloroplast or nucleus of a strain of algae, are known in the art. These methods have been described in a number of texts for standard molecular biological manipulation (for example, as described in Packer & Glaser, 1988, “Cyanobacteria”, Meth. Enzymol., Vol. 167: Weissbach & Weissbach, 1988, “Methods for plant molecular biology,” Academic Press, New York; Sambrook, Fritsch & Maniatis, 1989, “Molecular Cloning: A laboratory manual,” 2nd edition Cold Spring Harbor Laboratory Press, Cold Spring Harbor, N.Y.: Clark M. S., 1997, Plant Molecular Biology, Springer, N.Y.; WO 00/73455; Tan et al. J. Microbiol. 43: 361-365 (2005); Purton, Adv Exp Med Biol., 2007 616:34-45; Li et al., Gene, 2007 403(1-2):132-142; Leon et al., Adv Exp Med Biol, 2007 616:1-11; Newman et al., Genetics, 1990 126:875-888; and Steinbrenner et al., Applied and Environmental Microbiology, 2006 72(12):7477-7484). These methods include, for example, biolistic devices (for example, as described in Sanford, Trends In Biotech. (1988) 6: 299-302, and U.S. Pat. No. 4,945,050); electroporation (for example, as described in Fromm et al., Proc. Nat'l. Acad. Sci. USA (1985) 82: 5824-5828); use of a laser beam, vortexing with DNA treated glass beads (for example, as described in Kindle, Proc. Natl. Acad. Sciences USA 87: 1228-1232 (1990); and Newman et al., Genetics, 1990 126:875-888), microinjection, or any other method capable of introducing DNA into a host cell (e.g., an algal cell).

Nuclear transformation of eukaryotic algal cells can be by microprojectile mediated transformation, or can be by protoplast transformation, electroporation, introduction of DNA using glass fibers, or the glass bead agitation method. Non-limiting examples of nuclear transformation of eukaryotic algal cells are described in Kindle, Proc. Natl. Acad. Sciences USA 87: 1228-1232 (1990), and Shimogawara et al. Genetics 148: 1821-1828 (1998)).

Markers for nuclear transformation of algae include, without limitation, markers for rescuing auxotrophic strains (e.g., NIT1 and ARG7 in Chlamydomonas). Examples of markers for rescuing auxotrophic strains are also described in Kindle et al. J. Cell Biol. 109: 2589-2601 (1989), and Debuchy et al. EMBO J. 8: 2803-2809 (1989)). Examples of dominant selectable markers are CRY 1 and aada. Examples of dominant selectable markers are also described in Nelson et al. Mol. Cellular Biol. 14: 4011-4019 (1994), and Cerutti et al. Genetics 145: 97-110 (1997)). In some embodiments, the herbicide resistance gene is used as a selectable marker for transformants. A herbicide resistance gene can in some embodiments be co-transformed with a second gene encoding a protein to be produced by the alga (for example, a therapeutic protein, an industrial enzyme, or a protein that promotes or enhances production of a commercial, therapeutic, or nutritional product). The second gene, in some embodiments is provided on the same nucleic acid construct as the herbicide resistance gene for transformation into the alga, wherein the herbicide resistance gene is used as the selectable marker.

Plastid transformation can be by any method known to one skilled in the art for introducing a polynucleotide into a plant cell chloroplast. Examples of plastid transformation are described in U.S. Pat. Nos. 5,451,513, 5,545,817, 5,545,818, and International Publication No. WO 95/16783. In some embodiments, chloroplast transformation involves introducing regions of chloroplast DNA flanking a desired nucleotide sequence, allowing for homologous recombination of the exogenous DNA into the target chloroplast genome. In some embodiments, about one to about 1.5 kb flanking nucleotide sequences of chloroplast genomic DNA may be used. Using this method, point mutations in the chloroplast 16S rRNA and rps12 genes, which confer resistance to spectinomycin and streptomycin, may be utilized as selectable markers for transformation (Svab et al., Proc. Natl. Acad. Sci., USA 87:8526-8530, 1990). Microprojectile mediated transformation can be used to introduce a polynucleotide into an algal plant cell (Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996).

Transformation frequency may be increased by replacement of recessive rRNA or r-protein antibiotic resistance genes with a dominant selectable marker, including, but not limited to the bacterial aadA gene (Svab and Maliga, Proc. Natl. Acad. Sci., USA 90:913-917, 1993). Co-transformation with a second plasmid that confers resistance is also effective in selecting for transformants (Kindle et al. Proc. Natl. Acad. Sci., USA 88: 1721-1725 (1995)). It is apparent to one of skill in the art that a chloroplast may contain multiple copies of its genome, and therefore, the term “homoplasmic” or “homoplasmy” refers to the state where all copies of a particular locus of interest within a cell or organism are substantially identical. Plastid expression of genes inserted by homologous recombination into all of the multiple copies of the circular plastid genome present in each plant cell (the homoplastidic state) takes advantage of the enormous copy number advantage over nuclear-expressed genes to permit expression levels that can exceed 1%, 2%, 3%, 4%, 5%, 6%, 7%, 8%, 9%, or 10% of the total soluble plant protein.

Several cell division cycles following transformation are generally required to reach a homoplastidic state. Algae may be allowed to divide in the presence or absence of a selection agent (for example, kanamycin, spectinomycin, or streptomycin), or under stepped-up selection (use of a lower concentration of the selective agent than homoplastic cells would be expected to grow on, which can be increased over time) prior to screening transformants. Screening of transformants by PCR or Southern hybridization, for example, can be performed to determine whether a transformant is homoplastic or heteroplastic, and if heteroplastic, the degree to which the recombinant gene has integrated into copies of the chloroplast genome.

For nuclear or chloroplast transformation, a major benefit can be the utilization of a recombinant nucleic acid construct which contains both a selectable marker and one or more genes of interest. Typically, transformation of chloroplasts is performed by co-transformation of chloroplasts with two constructs: one containing a selectable marker and a second containing the gene(s) of interest. Transformants are screened for presence of the selectable marker (in some embodiments, a herbicide resistance gene) and, in some embodiments, for the presence of (a) further gene(s) of interest. Typically, secondary screening for one or more gene(s) of interest is performed by PCR or Southern blot (see, for example PCT/US2007/072465).

In other embodiments, two or more genes can be linked in a single nucleic acid construct for transformation into the chloroplast and insertion into the same locus. For example, two or more herbicide resistance genes, or one or more herbicide resistance genes and a gene encoding the Bt toxin, or one or more herbicide resistance genes and one or more genes encoding another polypeptide of interest, and a selectable marker gene, can be provided in the same nucleic acid construct flanked by chloroplast genome homology regions for linked integration into the chloroplast genome. The genes, in some embodiments, share regulatory regions, such as a promoter, 5′ UTR, and/or 3′UTR, for expression as an operon. In other embodiments, the genes do not share regulatory regions.

In some instances, a recombinant nucleic acid molecule is introduced into a chloroplast, wherein the recombinant nucleic acid molecule includes a first polynucleotide, which encodes at least one polypeptide (for example, 1, 2, 3, 4, or more polypeptides). In some embodiments, a polypeptide is operatively linked to a second, third, fourth, fifth, sixth, seventh, eighth, ninth, tenth and/or subsequent polypeptide. For example, several enzymes in a hydrocarbon production pathway may be linked, either directly or indirectly, such that products produced by one enzyme in the pathway, once produced, are in close proximity to the next enzyme in the pathway.

### Expression Vectors

The algae described herein can be transformed to modify the production of a product(s) with an expression vector, for example, to increase production of a product(s). The product(s) can be naturally produced by the algae or not naturally produced by the algae.

An expression vector can encode one or more heterologous nucleotide sequences (derived from an algae other than the host algae), one or more homologous nucleotide sequences (a sequence having homology to a host algae sequence), and/or one or more autologous nucleotide sequences (derived from the same algae). Homologous sequences are those that have at least 50%, 60%, 70%, 75%, 80%, 85%, 90%, or 95% homology to the sequence in the host algae. Examples of heterologous nucleotide sequences that can be transformed into an algal host cell include genes from bacteria, fungi, plants, photosynthetic bacteria, or other algae. Examples of autologous nucleotide sequences that can be transformed into an algal host cell include endogenous promoters and, for example, for chloroplast transformation, 5′ UTRs from the psbA, atpA, or rbcL genes. In some instances, a heterologous sequence is flanked by two autologous sequences or homologous sequences. In some instances, a heterologous sequence is flanked by two homologous sequences. The first and second homologous sequences can in some embodiments enable recombination of the heterologous sequence into the genome of the host organism or algae. The first and second homologous sequences can be at least about 100, about 200, about 300, about 400, about 500, about 1000, about 1500, about 2000, or about 2500 nucleotides in length.

In chloroplasts, regulation of gene expression generally occurs after transcription, and often during translation initiation. This regulation is dependent upon the chloroplast translational apparatus, as well as nuclear-encoded regulatory factors (for example, as described in Barkan and Goldschmidt-Clermont, Biochemie 82:559-572, 2000; and Zerges, Biochemie 82:583-601, 2000). The chloroplast translational apparatus generally resembles that of bacteria; chloroplasts contain 70S ribosomes; have mRNAs that lack 5′ caps and generally do not contain 3′ poly-adenylated tails (Harris et al., Microbiol. Rev. 58:700-754, 1994); and translation is inhibited in chloroplasts and in bacteria by selective agents such as chloramphenicol.

Some methods as described herein for transforming the chloroplast take advantage of proper positioning of a ribosome binding sequence (RBS) with respect to a coding sequence. It has previously been noted that such placement of an RBS results in robust translation in plant chloroplasts (for example, as described in U.S. Application 2004/0014174, published Jan. 20, 2004, incorporated herein by reference). Expression of polypeptides in chloroplasts does not proceed through cellular compartments typically traversed by polypeptides expressed from a nuclear gene and, therefore, are not subject to certain post-translational modifications such as glycosylation. As such, the polypeptides and protein complexes produced by some methods described herein can be expected to be produced without such post-translational modifications.

One or more codons of an encoding polynucleotide can be biased to reflect chloroplast and/or nuclear codon usage. Most amino acids are encoded by two or more different (degenerate) codons, and it is well recognized that various organisms utilize certain codons in preference to others. Such preferential codon usage, which also is utilized in chloroplasts, is referred to herein as “chloroplast codon usage”. The codon bias of the Chlamydomonas reinhardtii chloroplast genome has been reported (U.S. Application 2004/0014174). The nuclear codon bias of C. reinhardtii is also documented (Shao et al. Curr Genet. 53: 381-388 (2008)).

The term “biased,” when used in reference to a codon, means that the sequence of a codon in a polynucleotide has been changed such that the codon is one that is used preferentially in the target for which the bias is for, for example, alga cells and chloroplasts. A polynucleotide that is biased for chloroplast codon usage can be, for example, synthesized de novo, or can be genetically modified using routine recombinant DNA techniques, for example, by a site-directed mutagenesis method, to change one or more codons such that they are biased for chloroplast codon usage. Chloroplast codon bias can be variously skewed in different plants, including, for example, in alga chloroplasts as compared to tobacco. Generally, the chloroplast codon bias selected reflects chloroplast codon usage of the plant which is being transformed with the nucleic acids. For example, where C. reinhardtii is the host, the chloroplast codon usage is biased to reflect alga chloroplast codon usage (about 74.6% AT bias in the third codon position). In some embodiments, at least about 50% of the third nucleotide position of the codons are A or T. In other embodiments, at least 60%, 70%, 80%, 90%, or 99% of the third nucleotide position of the codons are A or T.

The nuclear genome of algae can also be codon biased, for example, the nuclear genome of Chlamydomonas reinhardtii is GC-rich and has a pronounced preference for G or C in the third position of codons (for example, as described in LeDizet and Piperno, Mol. Biol. Cell 6: 697-711 (1995); and Fuhrman et al. Plant Mol. Biol. 55: 869-881 (2004)).

One approach to construction of a genetically manipulated strain of alga involves transformation with a nucleic acid which encodes a gene of interest, for example, a herbicide resistance gene. In some embodiments, a transformation may introduce nucleic acids into the host alga cell (for example, a chloroplast or nucleus of a eukaryotic host cell). Transformed cells are typically plated on selective media (for example, containing kanamycin, hygromycin, and/or zeocin) following introduction of exogenous nucleic acids. This method may also comprise several steps for screening. Initially, a screen of primary transformants is typically conducted to determine which clones have proper insertion of the exogenous nucleic acids. Clones which show the proper integration may be replica plated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (for example, nested PCR and real time PCR). Particular examples of PCR are utilized in the examples described herein: however, one of skill in the art will recognize that other PCR techniques may be substituted for the particular protocols described. Following screening for clones with proper integration of exogenous nucleic acids, clones may be screened for the presence of the encoded protein. Protein expression screening typically is performed by Western blot analysis and/or enzyme activity assays, for example.

A recombinant nucleic acid molecule encoding a herbicide resistance gene can be contained in a vector. Furthermore, where the method is performed using a second (or more) recombinant nucleic acid molecules, the second recombinant nucleic acid molecule also can be contained in a vector, which can, but need not, be the same vector as that containing the first recombinant nucleic acid molecule. The vector can be any vector useful for introducing a polynucleotide into a host cell. In some instances, such as, but not limited, to transformation of some prokaryotic algae and the chloroplast of some eukaryotic algae, include a nucleotide sequence of host DNA or chloroplast genomic DNA that is sufficient to undergo homologous recombination with the host genomic DNA. For example, for chloroplast transformation, a nucleotide sequence comprising about 400 to about 1500 or more substantially contiguous nucleotides of chloroplast genomic DNA can be used as the homologous sequence. Chloroplast vectors and methods for selecting regions of a chloroplast genome for use as a vector are well known (for example, as described in Bock, J. Mol. Biol. 312:425-438 (2001); Staub and Maliga, Plant Cell 4:39-45 (1992); and Kavanagh et al., Genetics 152:1111-1122 (1999), each of which is incorporated herein by reference).

In some instances, such vectors include promoters. Promoters useful herein may come from any source (for example, viral, bacterial, fungal, protist, or animal). The promoters contemplated herein can be specific to photosynthetic organisms, non-vascular photosynthetic organisms, and/or algae, including photosynthetic bacteria. In some instances, the nucleic acids above are inserted into a vector that comprises a promoter of an algal species.

For chloroplast transformation, the promoter can be a promoter for expression in a chloroplast and/or other plastid. In some instances, the nucleic acids are chloroplast based. Examples of promoters contemplated for insertion of any of the nucleic acids herein into the chloroplast include those disclosed in US Application No. 2004/0014174, published Jan. 20, 2004. The promoter can be a constitutive promoter or an inducible promoter. A promoter typically includes necessary nucleic acid sequences near the start site of transcription, (for example, a TATA element).

The entire chloroplast genome of C. reinhardtii is available as GenBank Acec. No. BK000554 and is reviewed in J. Maul, et al. The Plant Cell 14: 2659-2679 (2002), both incorporated by reference herein. The Chlamydomonas genome is also provided to the public on the world wide web, at the URL “biology.duke.edu/chlamy_genome/-chlo ro.html” (Duke University) (see “view complete genome as text file” link and “maps of the chloroplast genome” link), each of which is incorporated herein by reference. Generally, the nucleotide sequence of the chloroplast genomic DNA is selected such that it is not contained in a portion of a gene that includes a regulatory sequence or coding sequence that, if disrupted due to a homologous recombination event, would produce a deleterious effect with respect to the chloroplast. Deleterious effects include, for example, effects on the replication of the chloroplast genome, or to a plant cell containing the chloroplast. In this respect, the website containing the C. reinhardtii chloroplast genome sequence also provides maps showing coding and non-coding regions of the chloroplast genome (also described in J. Maul, et al. The Plant Cell 14: 2659-2679 (2002)), thus facilitating selection of a sequence useful for constructing a vector. For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link: also accessible directly on world wide web at URL “biology.duke.edu/chlam-y/chloro/chlorol40.html”).

A vector utilized herein also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that a heterologous polynucleotide can be inserted into the vector and operatively linked to a desired regulatory element. The vector also can contain a prokaryote origin of replication (ori), for example, an E. coli ori or a cosmid ori, thus allowing passage of the vector in a prokaryote host cell, as well as in a plant chloroplast.

A regulatory element, as the term is used herein, broadly refers to a nucleotide sequence that regulates the transcription or translation of a polynucleotide or the localization of a polypeptide to which it is operatively linked. Examples include, but are not limited to, an RBS, a promoter, an enhancer, a transcription terminator, an initiation (start) codon, a splicing signal for intron excision and maintenance of a correct reading frame, a STOP codon, an amber or ochre codon, and an IRES. Another example of a regulatory element is a cell compartmentalization signal (for example, a sequence that targets a polypeptide to the cytosol, nucleus, mitochondria, chloroplast, chloroplast membrane, or cell membrane). Such signals are well known in the art and have been widely reported (for example, as described in U.S. Pat. No. 5,776,689).

Any of the expression vectors herein can further comprise a regulatory control sequence. A regulatory control sequence may include for example, promoter(s), operator(s), repressor(s), enhancer(s), transcription termination sequence(s), sequence(s) that regulate translation, and/or other regulatory control sequence(s) that are compatible with the host cell and control the expression of the nucleic acid molecule(s). In some cases, a regulatory control sequence includes transcription control sequence(s) that are able to control, modulate, or effect the initiation, elongation, and/or termination of transcription. For example, a regulatory control sequence can increase the transcription and/or translation rate and/or the efficiency of a gene or gene product in an organism, wherein expression of the gene or gene product is upregulated, resulting (directly or indirectly) in the increased production of the desired product. The regulatory control sequence may also result in the increase of production of a protein by increasing the stability of the related gene.

A regulatory control sequence can be autologous or heterologous, and if heterologous, may have homology to a sequence in the host alga. For example, a heterologous regulatory control sequence may be derived from another species of the same genus of the organism (for example, another algal species). In another example, an autologous regulatory control sequence can be derived from an organism in which an expression vector is to be expressed. Depending on the application, regulatory control sequences can be used that effect inducible or constitutive expression. For example, the algal regulatory control sequences can be used, and can be of nuclear, viral, extrachromosomal, mitochondrial, or chloroplastic origin. A regulatory control sequence can be chimeric, having sequences from the regulatory region of two or more different genes, and/or can include mutated variants of regulatory control sequences of genes or can include synthetic sequences.

Suitable regulatory control sequences include those naturally associated with the nucleotide sequence to be expressed (for example, an algal promoter operably linked with an algal-derived nucleotide sequence in nature). Suitable regulatory control sequences include regulatory control sequences not naturally associated with the nucleic acid molecule to be expressed (for example, an algal promoter of one species operatively linked to a nucleotide sequence of another organism or algal species). The latter regulatory control sequences can be a sequence that controls expression of another gene within the same species (for example, autologous) or can be derived from a different organism or species (for example, heterologous).

To determine whether a putative regulatory control sequence is suitable, the putative regulatory control sequence is linked to a nucleic acid molecule typically encoding a protein that produces an easily detectable signal. A construct comprising the putative regulatory control sequence and nucleic acid molecule may then be introduced into an alga or other organism by standard techniques and expression thereof is monitored. For example, if the nucleic acid molecule encodes a dominant selectable marker, the alga or organism to be used is tested for the ability to grow in the presence of a compound for which the marker provides resistance. Examples of such selectable markers include the genes encoding kanamycin, zeocin, or hygromycin.

In some cases, a regulatory control sequence is a promoter, such as a promoter adapted for expression of a nucleotide sequence in a non-vascular, photosynthetic organism. For example, the promoter may be an algal promoter, for example as described in U.S. Publ. Appl. Nos. 2006/0234368, now U.S. Pat. No. 7,449,568, issued Nov. 11, 2008 and 2004/0014174, published Jan. 20, 2004, and in Hallmann, Transgenic Plant J. 1:81-98 (2007). The promoter may be a chloroplast specific promoter or a nuclear promoter. A regulatory control sequence herein can be found in a variety of locations, including for example, coding and non-coding regions, 5′ untranslated regions (for example, regions upstream from the coding region), and 3′ untranslated regions (for example, regions downstream from the coding region). Thus, in some instances an autologous or heterologous nucleotide sequence can include one or more 3′ or 5′ untranslated regions, one or more introns, and/or one or more exons.

For example, in some embodiments, a regulatory control sequence can comprise a Cyclotella cryptica acetyl-CoA carboxylase 5′ untranslated regulatory control sequence or a Cyclotella cryptica acetyl-CoA carboxylase 3′-untranslated regulatory control sequence (for example, as described in U.S. Pat. No. 5,661,017).

A regulatory control sequence may also encode a chimeric or fusion polypeptide, such as protein AB, or SAA, that promote the expression of heterologous nucleotide sequences and proteins. Other regulatory control sequences include autologous intron sequences that may promote translation of a heterologous sequence.

The regulatory control sequences used in any of the expression vectors described herein may be inducible. Inducible regulatory control sequences, such as promoters, can be inducible by light, for example. Regulatory control sequences may also be autoregulatable. Examples of autoregulatable regulatory control sequences include those that are autoregulated by, for example, endogenous ATP levels or by the product produced by the algae. In some instances, the regulatory control sequences may be inducible by an exogenous agent. Other inducible elements are well known in the art and may be adapted for use as described herein.

The promoter can be a promoter for expression in the nucleus of an alga. Examples of C. reinhardtii promoters contemplated for use with any of the nucleic acids described herein include, but are not limited to, the RBCS2 promoter, the HSP70A-RBCS2 tandem promoter (for example, as described in Lodha et al. Euk. Cell 7: 172-176 (2008), and the PSAD promoter. The promoter can be a constitutive promoter or an inducible promoter. Examples of inducible promoters of C. reinhardtii include the NIT1 promoter, the CYC6 promoter (Ferrante et al. PLoS ONE, 3: 1-8 (2008)), and the CA1 promoter. A construct for nuclear transformation can also, in some embodiments, include at least one intron, for example, the Rb-int intron that increases expression of a gene of interest (Lambreras et al. Plant J 14: 441-447 (1998)).

Various combinations of the regulatory control sequences described herein may be combined with other features described herein. In some cases, an expression vector comprises one or more regulatory control sequences operatively linked to a nucleotide sequence encoding a polypeptide that, for example, upregulates production of a product described herein.

A vector or other recombinant nucleic acid molecule may include a nucleotide sequence encoding a reporter polypeptide or other selectable marker. The term “reporter” or “selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by the eye or using appropriate instrumentation (for example, as described in Giacomin, Plant Sci. 116:59-72, 1996; Scikantha, J. Bacteriol. 178:121, 1996; Gerdes, FEBS Lett. 389:44-47, 1996; and Jefferson, EMBO J. 6:3901-3907, 1987, beta-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can be used to select prokaryotic cells, and/or plant cells that express the marker and, therefore, can be useful as a component of a vector (for example, as described in Bock, J. Mol. Biol. 312:425-438 (2001)). Examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which allows cells to utilize mannose (for example, as described in WO 94/20627): ornithine decarboxylase, which confers resistance to the ornithine decarboxylase inhibitor, 2-(difluoromethyl)-DL-omithine (DFMO) (for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Selectable markers include polynucleotides that confer dihydrofolate reductase (DHFR) or neomycin resistance for eukaryotic cells. Suitable markers also include polynucleotides that confer resistance to tetracycline: ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide, and sulfonylurea resistance in plants (for example, as described in Maliga et al., Methods in Plant Molecular Biology, Cold Spring Harbor Laboratory Press, 1995, page 39).

Herbicide resistance genes can also be used as selectable markers. The host algae can be transformed with polynucleotides encoding one or more proteins that confer resistance to a herbicide(s), and be selected with the herbicide(s) the encoded protein confers resistance to. Alternatively, a selectable marker such as kanamycin, bleomycin, or nitrate reductase may be co-transformed with the herbicide resistance marker, and transformed cells can initially be selected for using a selection media or compound that is not related to the herbicide resistance gene.

Reporter genes have been successfully used in chloroplasts of higher plants, and high levels of recombinant protein expression have been shown. In addition, reporter genes have been used in the chloroplast of C. reinhardtii. Reporter genes greatly enhance the ability to monitor gene expression in a number of biological organisms. In chloroplasts of higher plants, for example, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransf-erase (aadA, for example, as described in Svab and Maliga, Proc. Natl. Acad. Sci., USA 90:913-917, 1993), and the Aequorea victoria GFP (for example, as described in Sidorov et al., Plant J. 19:209-216, 1999) have been used as reporter genes. Various reporter genes are also described in a review by Heifetz, Biochemie 82:655-666, 2000, on the genetic engineering of the chloroplast. Each of these genes has attributes that make them useful reporters of chloroplast gene expression, such as case of analysis, sensitivity, or the ability to examine expression in situ. Several reporter genes have been expressed in the chloroplast of the eukaryotic green alga, C. reinhardtii, including, for example, aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet 263:404-410, 2000).

In some instances, the vectors will contain elements such as an E. coli or S. cerevisiae origin of replication. Such features, combined with appropriate selectable markers, allows for the vector to be “shuttled” between the target host cell and the bacterial and/or yeast cell. The ability to passage a shuttle vector in a secondary host may allow for more convenient manipulation of the features of the vector. For example, a reaction mixture containing the vector and putative inserted polynucleotides of interest can be transformed into prokaryote host cells such as E. coli, amplified, collected using routine methods, and examined to identify vectors containing an insert or construct of interest. If desired, the vector can be further manipulated, for example, by performing site-directed mutagenesis of the inserted polynucleotide, then again amplifying and selecting vectors having the mutated polynucleotide of interest. A shuttle vector then can be introduced into algal cells, wherein a polypeptide of interest can be expressed and, if desired, isolated.

### Herbicides and Herbicide Resistance Genes

The herbicide resistant algae provided herein are transformed with polynucleotides that encode a protein that confers resistance to a herbicide. Herbicide resistance allows for the growth of the algal host species in a concentration of herbicide that prevents the growth of untransformed algae of the same species.

In some embodiments, the herbicide to which the transformed alga is resistant is a herbicide that inhibits amino acid biosynthesis. In some embodiments, the herbicide is a herbicide that inhibits carotenoid biosynthesis. In other embodiments, the herbicide is not a herbicide that inhibits carotenoid biosynthesis. In some embodiments, the herbicide is a herbicide that inhibits photosynthesis. In other embodiments, the herbicide is not a herbicide that inhibits photosynthesis. In some embodiments, the herbicide is a photosensitizer or photobleacher. In other embodiments, the herbicide is not a photosensitizer or photobleacher. In some embodiments, the herbicide is an antibiotic. In other embodiments, the herbicide is not an antibiotic. In some embodiments, the herbicide is not a herbicide that inhibits amino acid biosynthesis, or is not a herbicide that inhibits photosystem II.

The herbicide inhibits growth of the host algal species that is not transformed with the gene conferring herbicide resistance, and also inhibits the growth of one or more other algal species. In some embodiments, the herbicide is effective against one or more bacterial species. In some embodiments, the herbicide is effective against one or more fungal species. In some embodiments, the herbicide to which the alga is resistant is a broad spectrum herbicide, and prevents the growth of many species of vascular plants.

A herbicide resistance gene as used herein is a gene that encodes resistance to any type of herbicide that inhibits the growth of the nontransformed host alga, including, but not limited to, herbicides that inhibit amino acid biosynthesis, herbicides that inhibit carotenoid biosynthesis, herbicides that inhibit fatty acid biosynthesis, herbicides that inhibit photosynthesis, and photobleaching agents. In some embodiments, a protein encoded by a herbicide resistance gene confers resistance to an antibiotic (where an antibiotic is a compound that is made by a microorganism that inhibits the growth of bacteria, or a compound synthesized based on the structures of bacterial growth-inhibiting compounds made by microorganisms, such as for example, spectinomycin, kanamycin, or fosmidomycin). In some embodiments, a protein that confers resistance to a herbicide is not a protein that confers resistance to an antibiotic. In some embodiments, resistance to a particular herbicide is conferred by multiple proteins. In some embodiments, resistance to a particular herbicide is conferred by a single protein.

Mechanisms of herbicide resistance are also varied. Herbicide resistance of a host alga can be, for example, by transformation of the host alga with a gene that leads to: the production of a protein that inactivates the herbicide; to the production of mutant forms of a protein targeted by the herbicide, such that the mutant form is not affected, or less affected, by the herbicide than its wild-type counterpart; to the production of large amounts of an enzyme or other biomolecule to compensate for the effects of the herbicide; to the production of an enzyme or other biomolecule that ameliorates or remedies the effects of the herbicide, or to the production of a protein that prevents transport of the herbicide into the cell. The following discussion of herbicides does not limit the methods, vectors, polynucleotides, constructs, or algal genomes disclosed herein to those encoding the particular disclosed proteins that confer herbicide resistance. In addition, the following discussion does not in any way restrict the herbicide resistance genes, polynucleotides, or nucleic acid constructs that can be used for conferring herbicide resistance in algae.

In some embodiments, a herbicide resistance gene confers resistance to a herbicide that inhibits amino acid biosynthesis. Examples of such herbicides are glyphosate that inhibits aromatic amino acid synthesis, and imidazolamine that inhibits branched chain amino acid synthesis. Due to common amino acid biosynthesis pathways in plants and many bacteria and fungi, such herbicides in many instances prevent the growth of bacterial and/or fungal species.

The low toxicity of the herbicide glyphosate is due in part to the fact that it targets a biosynthetic pathway for aromatic amino acids that is not present in animals. The inhibition by glyphosate of 5-enolpyruvylshikimate-3-phosphate synthase, an enzyme used in aromatic amino acid synthesis in bacteria, some fungi, and plants (including algae), leads to the death of the organism. Genes conferring resistance to glyphosate that can be used to transform algae include mutant forms of Class I EPSPS genes that occur in eukaryotes (for example, as described in U.S. Pat. Nos. 4,971,908, 5,310,667, and 5,866,775), as well as glyphosate resistant forms of Class II EPSPS genes found in prokaryotes (for example, those disclosed in U.S. Pat. No. 5,627,061 and U.S. Pat. No. 5,633,435) that encode EPSPS proteins that in may be more catalytically active than herbicide resistant forms of Class I EPSPS. Recently discovered EPSPS genes that confer resistance to glyphosate that do not belong to either Class I or Class II (non-Class I/Class II EPSP genes) include those isolated from environmental samples (for example, as described in U.S. Pat. Nos. 7,238,508 and 7,214,535). Resistance to glyphosate can also be conferred by transformation of a host organism or algae with any combination of one or more EPSPS Class I, Class II, or non-Class I/Class II genes, or operatively linked to nucleic acids sequences that promote their overexpression in the host cells. Other proteins that confer resistance to glyphosate include glutathione oxidoreductase (“GOX”: for example, as described in WO 92/00377) and glutathione acetyltransferase “GAT” (for example, as described in Castle et al. Science 304: 1151-1154 (2004)). An algal host in some embodiments can be transformed with a gene encoding encoding GAT and/or a gene encoding GOX in addition to a gene encoding a glyphosate resistant EPSPS.

Other herbicides that target amino acid biosynthetic pathways include sulfonylureas, imidazolidones, and 1,2,4-triazol pyrimidines that inhibit acetolactate synthase (ALS; also called acctohydroxyacid synthase, or AHAS, that participates in the synthesis of branched chain amino acids), and phosphinothricin (also called glufosinate) which inhibits glutamine synthase. Both sulfonylureas and phosphinothricin are also effective against some bacteria and fungi. Genes conferring resistance to sulfonylureas include a mutant prokaryotic ALS gene from E. coli (for example, as described in Yadav et al. Proc Natl Acad Sci. USA 83: 4418-4422 (1986)) as well as a mutant ALS genes from yeast (for example, as described in Falco et al. Genetics 109: 21-35 (1985)), tobacco (for example, as described in Lee et al. EMBO J 7: 1241-1248 (1988)), and Chlamydomonas (for example, as described in Hartnett et al. Plant Physiol. 85: 898-901 (1987): and Kovar et al., The Plant J. 29: 109-117 (2002)). Genes conferring resistance to phosphinothricin include the phosphinothricin acetyltransferase or bar gene, (for example, as described in White et al., Nucl. Acids Res. 18:1062, 1990: and Spencer et al., Theor. Appl. Genet. 79:625-631, 1990).

Several herbicides interfere with carotenoid synthesis. Carotenoid synthesis-inhibiting herbicides include aminotriazole, pyridazinones, m-phenoxybenzamides, fluridone, difunone, and 4-hydroxypyridines. In some instances, the lethal effects of inhibiting carotenoid synthesis are prevented by overexpression of enzymes of the terpenoid synthesis pathway. Mutant forms of genes of the carotenoid synthesis pathway such as, for example, phytoene desaturase, that confer herbicide resistance are also known (for example, as described in Steinbrenner and Sandmann, Applied and Environ Microbiology 72: 7477-7484).

Still another class of herbicides binds the photosystem II reaction center D1 protein (product of the psbA gene, encoded in the chloroplast genome of plants). Herbicides that bind D1 and inhibit photosynthesis include atrazine, diuron, anilides, benzimidazoles, biscarbamate, pyrimadazinones, triazinediones, triazines, triazinones, uracils, substituted ureas, quinones, and hydroxybenzonitriles. Mutant forms of the psbA gene that encode proteins that do not bind atrazine are known in many organisms, including cyanobacterial species and Chlamydomonas (for example, as described in Golden and Haselkorn Science 229: 1104-1107 (1985); Przibila et al. The Plant Cell 3: 169-174 (1991); and Erickson et al. Proc. Natl. Acad. Sci. USA 81: 3617-3621 (1984)).

The halogenated hydrobenzonitrile herbicides (e.g., bromoxynil) also inhibit photosystem II. Bromoxynil nitrilase (for example, as described in U.S. Pat. No. 4,810,648; and Stalker et al. Science 242: 419-423 (1988)) confers herbicide resistance by converting bromoxynil to a nontoxic compound.

Yet another type of herbicide is known as a “photo-oxidizer” or “photobleacher”. Such herbicides include the bipyridyliums diquat and paraquat that accept electrons from photosystem 1 and generate superoxide radicals. It has been reported that overexpression of anti-oxidant proteins such as glutathione reductase, superoxide dismutase, and a fusion protein of cytochrome P450-superoxide dismutase can reduce the effects of such photo-oxidizers. Other photobleaching herbicides are the p-nitrodiphenylethers, the oxadiazoles, and the N-phenylimides. These compounds inhibit protoporphyrin oxidase, causing accumulation of protoporphyrin IX, a photo-oxidizer. A gene encoding a mutant form of protoporphyrin oxidase that confers resistance to porphyric herbicides has been identified in Chlamydomonas (Randolph-Anderson et al. Plant Mol Biol. 38: 839-59 (1998)).

Herbicides that inhibit multidomain eukaryotic-type acetyl-CoA carboxylase (ACCase), an enzyme necessary for de novo fatty acid biosynthesis, are effective against some plant species. For example, aryloxyphenoxy propionates (e.g., diclofop, diclofop-methyl, clodinafop, clodimafop-propargyl, cyhalofop, cyhalofop-butyl, fenoxaprop, fenoxaprop-P-ethyl, fluazifop, fluazipfop-butyl, fluazifop-P-butyl, haloxyfop, propaquizafop, quizalofop, and quizalofop-P) and cyclohexandione oxime herbicides (e.g., alloxydim, tralkoxydim, tepraloxydim, butroxydim, cycloxydim, sethoxydim, clethodim, and BAS 625 H) are lethal to plants that lack a prokaryotic-type ACCase, and may interfere with the reproduction of some insects (for example, as described in WO 04/060058). Genes conferring resistance to these herbicides include genes encoding the subunits of a prokaryotic-type acetyl-CoA carboxylase, as well as genes encoding mutant forms of a eukaryotic-type acetyl-CoA carboxylase, such as, for example, the ACCase gene from herbicide-resistant maize and the ACCase gene from herbicide-resistant Lolium rigidum (for example, as described in Zagnitko et al. Proc Natl Acad Sci USA 98: 6617-6622 (2001)).

### Nucleic Acid Sequences for Use in the Embodiments of the Disclosure

Exemplary nucleic acid sequences for use in the present disclosure are:

(a) the nucleotide sequence of SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO:100;
(b) a nucleotide sequence homologous to SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO:100; or
(c) the nucleotide sequence of SEQ ID NO: 5, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 56, SEQ ID NO: 57, SEQ ID NO: 60, SEQ ID NO:63, SEQ ID NO:64, SEQ ID NO:66, SEQ ID NO:67, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:93, SEQ ID NO:94, SEQ ID NO:95, SEQ ID NO:97, SEQ ID NO:98, or SEQ ID NO:100, comprising one or more mutations.

Mutations can be point mutations, deletions, insertions or any other type of mutation or alteration know to one of skill in the art. Homologous sequences can be, for example, about 70% homologous, about 75% homologous, about 80% homologous, about 85% homologous, about 90% homologous, about 95% homologous, or about 99% homologous. Homologous sequences can be, for example, more than 70% homologous, more than 75% homologous, more than 80% homologous, more than 85% homologous, more than 90% homologous, more than 95% homologous, or more than 99% homologous.

### Protein Sequences for Use in the Embodiments of the Disclosure

Exemplary amino acid sequences for use in the present disclosure are:

(a) the amino acid sequence of SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO:11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99;
(b) an amino acid sequence homologous to SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; or
(c) the amino acid sequence of SEQ ID NO: 1, SEQ ID NO: 2, SEQ ID NO: 3, SEQ ID NO: 4, SEQ ID NO: 6, SEQ ID NO: 7, SEQ ID NO: 9, SEQ ID NO:11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 58, SEQ ID NO: 59, SEQ ID NO:61, SEQ ID NO:62, SEQ ID NO:65, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:96, or SEQ ID NO:99; comprising one or more mutations.

Mutations can be point mutations, deletions, insertions or any other type of mutation or alteration know to one of skill in the art. Homologous sequences can be, for example, about 70% homologous, about 75% homologous, about 80% homologous, about 85% homologous, about 90% homologous, about 95% homologous, or about 99% homologous. Homologous sequences can be, for example, more than 70% homologous, more than 75% homologous, more than 80% homologous, more than 85% homologous, more than 90% homologous, more than 95% homologous, or more than 99% homologous.

Some of the sequences listed herein have addition amino acids or nucleic acids at the beginning of the sequence as a result of cloning. For example, some of the sequences have a Met at the beginning. One skilled in the art would understand this and be able to remove the unwanted sequences without undue experimentation.

SEQ ID NO: 1 is the amino acid sequence of the C. reinhardtii EPSPS cDNA.

SEQ ID NO: 2 is the amino acid sequence of the C. reinhardtii EPSPS with the double mutations G163A and A252T.

SEQ ID NO: 3 is the amino acid sequence of the Agrobacterium sp. Strain CP4 EPSPS

SEQ ID NO: 4 is the amino acid sequence of the Synechococcus elongates PCC 7942 Phytoene desaturase.

SEQ ID NO: 5 is the nucleotide sequence of an EPSPS open reading frame from U.S. Pat. No. 7,238,508

SEQ ID NO: 6 is the amino acid sequence of SEQ ID NO: 5.

SEQ ID NO: 7 is the amino acid sequence of the Petunia x hybrida EPSPS

SEQ ID NO: 8 is the C. reinhardtii chloroplast genome codon-optimized nucleotide sequence of wildtype E. coli EPSPS with an additional 9 nucleotides on the 5′ end and an added 3′ sequence encoding for an affinity tag.

SEQ ID NO: 9 is the amino acid sequence of SEQ ID NO: 8

SEQ ID NO: 10 is the C. reinhardtii chloroplast genome codon-optimized nucleotide sequence of mutated E. coli EPSPS encoding for the G96A mutation with an additional 9 nucleotides on the 5′ end and an added 3′ sequence encoding for an affinity tag.

SEQ ID NO: 11 is the amino acid sequence of SEQ ID NO: 10

SEQ ID NO: 12 is the C. reinhardtii chloroplast genome codon-optimized nucleotide sequence of mutated E. coli EPSPS encoding for the A183T mutation with an additional 9 nucleotides on the 5′ end and an added 3′ sequence encoding for an affinity tag.

SEQ ID NO: 13 is the amino acid sequence of SEQ ID NO: 12

SEQ ID NO: 14 is the C. reinhardtii chloroplast genome codon-optimized nucleotide sequence of mutated E. coli EPSPS encoding for the G96A and A183T mutations with an additional 9 nucleotides on the 5′ end and an added 3′ sequence encoding for an affinity tag.

SEQ ID NO: 15 is the amino acid sequence of SEQ ID NO: 14

SEQ ID NO: 16 is the C. reinhardtii chloroplast genome codon-optimized nucleotide sequence of mature (without the sequence encoding the predicted chloroplast targeting peptide) wildtype C. reinhardtii EPSPS cDNA with an additional 9 nucleotides on the 5′ end and an added 3′ sequence encoding for an affinity tag.

SEQ ID NO: 17 is the amino acid sequence of SEQ ID NO: 16

SEQ ID NO: 18 is the C. reinhardtii chloroplast genome codon-optimized nucleotide sequence of mature (without the sequence encoding the predicted chloroplast targeting peptide) and mutated C. reinhardtii EPSPS cDNA encoding for the G163A (based on SEQ ID NO: 1) mutation with an additional 9 nucleotides on the 5′ end and an added 3′ sequence encoding for an affinity tag.

SEQ ID NO: 19 is the amino acid sequence of SEQ ID NO: 18

SEQ ID NO: 20 is the C. reinhardtii chloroplast genome codon-optimized nucleotide sequence of mature (without the sequence encoding the predicted chloroplast targeting peptide) and mutated C. reinhardtii EPSPS cDNA encoding for the A252T (based on SEQ ID NO: 1) mutation with an additional 9 nucleotides on the 5′ end and an added 3′ sequence encoding for an affinity tag.

SEQ ID NO: 21 is the amino acid sequence of SEQ ID NO: 20

SEQ ID NO: 22 is the C. reinhardtii chloroplast genome codon-optimized nucleotide sequence of mature (without the sequence encoding the predicted chloroplast targeting peptide) and mutated C. reinhardtii EPSPS cDNA encoding for the G163A and A252T (based on SEQ ID NO: 1) mutations with an additional 9 nucleotides on the 5′ end and an added 3′ sequence encoding for an affinity tag.

SEQ ID NO: 23 is the amino acid sequence of SEQ ID NO: 22

SEQ ID NO: 24 is the nucleotide sequence of the wildtype precursor (with the 5′ sequence encoding the chloroplast targeting peptide) C. reinhardtii EPSPS cDNA with an additional 9 nucleotides on the 5′ end and an added 3′ sequence encoding for an affinity tag.

SEQ ID NO: 25 is the amino acid sequence of SEQ ID NO: 24

SEQ ID NO: 26 is the nucleotide sequence of the mutated precursor (with the 5′ sequence encoding the chloroplast targeting peptide) C. reinhardtii EPSPS cDNA encoding for the G163A (based on SEQ ID NO: 1) mutation with an additional 9 nucleotides on the 5′ end and an added 3′ sequence encoding for an affinity tag.

SEQ ID NO: 27 is the amino acid sequence of SEQ ID NO: 26

SEQ ID NO: 28 is the nucleotide sequence of the mutated precursor (with the 5′ sequence encoding the chloroplast targeting peptide) C. reinhardtii EPSPS cDNA encoding for the A252T (based on SEQ ID NO: 1) mutation with an additional 9 nucleotides on the 5′ end and an added 3′ sequence encoding for an affinity tag.

SEQ ID NO: 29 is the amino acid sequence of SEQ ID NO: 28

SEQ ID NO: 30 is the nucleotide sequence of the mutated precursor (with the 5′ sequence encoding the chloroplast targeting peptide) C. reinhardtii EPSPS cDNA encoding for the G163A and

A252T (based on SEQ ID NO: 1) mutations with an additional 9 nucleotides on the 5′ end and an added 3′ sequence encoding for an affinity tag.

SEQ ID NO: 31 is the amino acid sequence of SEQ ID NO: 30

SEQ ID NO: 32 is the nucleotide sequence of the wildtype C. reinhardtii EPSPS genomic DNA (amplified from nuclear genome) with an added 3′ sequence encoding for an affinity tag.

SEQ ID NO: 33 is the amino acid sequence of SEQ ID NO: 32

SEQ ID NO: 34 is the nucleotide sequence of the mutated C. reinhardtii EPSPS genomic DNA (amplified from nuclear genome) encoding for the G163A (based on SEQ ID NO: 1) mutation with an added 3′ sequence encoding for an affinity tag.

SEQ ID NO: 35 is the amino acid sequence of SEQ ID NO: 34

SEQ ID NO: 36 is the nucleotide sequence of the mutated C. reinhardtii EPSPS genomic DNA (amplified from nuclear genome) encoding for the A252T (based on SEQ ID NO: 1) mutation with an added 3′ sequence encoding for an affinity tag.

SEQ ID NO: 37 is the amino acid sequence of SEQ ID NO: 36

SEQ ID NO: 38 is the nucleotide sequence of the mutated C. reinhardtii EPSPS genomic DNA (amplified from nuclear genome) encoding for the G163A and A252T (based on SEQ ID NO: 1) mutations with an additional sequence on the 3′ end encoding for an affinity tag.

SEQ ID NO: 39 is the amino acid sequence of SEQ ID NO: 38

SEQ ID NO: 40 is the amino acid sequence of SEQ ID NO: 68 with an additional three residues on the N-terminus as a result of the cloning.

SEQ ID NO: 41 is the amino acid sequence of SEQ ID NO: 70 with an additional three residues on the N-terminus as a result of the cloning.

SEQ ID NO: 42 is the amino acid sequence of SEQ ID NO: 72 with an additional three residues on the N-terminus as a result of the cloning.

SEQ ID NO: 43 is the amino acid sequence of SEQ ID NO: 74 with an additional three residues on the N-terminus as a result of the cloning.

SEQ ID NO: 44 is the amino acid sequence of SEQ ID NO: 76 with an additional three residues on the N-terminus as a result of the cloning.

SEQ ID NO: 45 is the amino acid sequence of SEQ ID NO: 78 with an additional three residues on the N-terminus as a result of the cloning.

SEQ ID NO: 46 is the amino acid sequence of SEQ ID NO: 80 with an additional three residues on the N-terminus as a result of the cloning.

SEQ ID NO: 47 is the amino acid sequence of SEQ ID NO: 82 with an additional three residues on the N-terminus as a result of the cloning.

SEQ ID NO: 48 is the amino acid sequence of SEQ ID NO: 84 with an additional three residues on the N-terminus as a result of the cloning.

SEQ ID NO: 49 is the amino acid sequence of SEQ ID NO: 86 with an additional three residues on the N-terminus as a result of the cloning.

SEQ ID NO: 50 is the amino acid sequence of SEQ ID NO: 88 with an additional three residues on the N-terminus as a result of the cloning.

SEQ ID NO: 51 is the amino acid sequence of SEQ ID NO: 90 with an additional three residues on the N-terminus as a result of the cloning.

SEQ ID NO: 52 is the amino acid sequence of SEQ ID NO: 92.

SEQ ID NO: 53 is the amino acid sequence of SEQ ID NO: 93.

SEQ ID NO: 54 is the amino acid sequence of SEQ ID NO: 94.

SEQ ID NO: 55 is the amino acid sequence of SEQ ID NO: 95.

SEQ ID NO: 56 is the C. reinhardtii chloroplast genome codon-optimized nucleotide sequence of SEQ ID NO: 3.

SEQ ID NO: 57 is the nucleotide sequence encoding SEQ ID NO: 4.

SEQ ID NO: 58 is the amino acid sequence of the mature (without the predicted chloroplast targeting peptide) C. reinhardtii EPSPS.

SEQ ID NO: 59 is the amino acid sequence of wildtype T. viride cellobiohydrolase I.

SEQ ID NO: 60 is the C. reinhardtii chloroplast genome codon-optimized nucleotide sequence of SEQ ID NO: 59.

SEQ ID NO: 61 is the amino acid sequence of wildtype C. reinhardtii acetolactate synthase large subunit.

SEQ ID NO: 62 is the amino acid sequence of the wildtype mature (without the predicted chloroplast targeting peptide) C. reinhardtii acetolactate synthase large subunit with an additional N-terminal methionine and a C-terminal affinity tag.

SEQ ID NO: 63 is the C. reinhardtii chloroplast genome codon-optimized nucleotide sequence of SEQ ID NO: 62.

SEQ ID NO: 64 is the C. reinhardtii chloroplast genome codon-optimized nucleotide sequence of the mature (without the predicted chloroplast targeting peptide) and mutated C. reinhardtii acetolactate synthase large subunit encoding for the P198S, W580L, and G666I (based on SEQ ID NO: 61) mutations with an additional 5′ start codon and an added 3′ sequence encoding for an affinity tag.

SEQ ID NO: 65 is the amino acid sequence of SEQ ID NO: 64.

SEQ ID NO: 66 is the nucleotide sequence of the wildtype E. coli EPSPS.

SEQ ID NO: 67 is the nucleotide sequence of the mutated E. coli EPSPS encoding for the G96A and A183T mutations and an added 3′ sequence encoding for an affinity tag.

SEQ ID NO: 68 is SEQ ID NO: 8 without the additional nucleotides on both the 5′ and 3′ ends.

SEQ ID NO: 69 is the amino acid sequence of SEQ ID NO: 68.

SEQ ID NO: 70 is SEQ ID NO: 10 without the additional nucleotides on both the 5′ and 3′ ends.

SEQ ID NO: 71 is the amino acid sequence of SEQ ID NO: 70.

SEQ ID NO: 72 is SEQ ID NO: 12 without the additional nucleotides on both the 5′ and 3′ ends.

SEQ ID NO: 73 is the amino acid sequence of SEQ ID NO: 72.

SEQ ID NO: 74 is SEQ ID NO: 14 without the additional nucleotides on both the 5′ and 3′ ends.

SEQ ID NO: 75 is the amino acid sequence of SEQ ID NO: 74.

SEQ ID NO: 76 is SEQ ID NO: 16 without the additional nucleotides on both the 5′ and 3′ ends.

SEQ ID NO: 77 is the amino acid sequence of SEQ ID NO: 76.

SEQ ID NO: 78 is SEQ ID NO: 18 without the additional nucleotides on both the 5′ and 3′ ends.

SEQ ID NO: 79 is the amino acid sequence of SEQ ID NO: 78.

SEQ ID NO: 80 is SEQ ID NO: 20 without the additional nucleotides on both the 5′ and 3′ ends.

SEQ ID NO: 81 is the amino acid sequence of SEQ ID NO: 80.

SEQ ID NO: 82 is SEQ ID NO: 22 without the additional nucleotides on both the 5′ and 3′ ends.

SEQ ID NO: 83 is the amino acid sequence of SEQ ID NO: 82.

SEQ ID NO: 84 is SEQ ID NO: 24 without the additional nucleotides on both the 5′ and 3′ ends.

SEQ ID NO: 85 is the amino acid sequence of SEQ ID NO: 84.

SEQ ID NO: 86 is SEQ ID NO: 26 without the additional nucleotides on both the 5′ and 3′ ends.

SEQ ID NO: 87 is the amino acid sequence of SEQ ID NO: 86.

SEQ ID NO: 88 is SEQ ID NO: 28 without the additional nucleotides on both the 5′ and 3′ ends.

SEQ ID NO: 89 is the amino acid sequence of SEQ ID NO: 88.

SEQ ID NO: 90 is SEQ ID NO: 30 without the additional nucleotides on both the 5′ and 3′ ends.

SEQ ID NO: 91 is the amino acid sequence of SEQ ID NO: 90.

SEQ ID NO: 92 is SEQ ID NO: 32 without the additional nucleotides on the 3′ end.

SEQ ID NO: 93 is SEQ ID NO: 34 without the additional nucleotides on the 3′ end.

SEQ ID NO: 94 is SEQ ID NO: 36 without the additional nucleotides on the 3′ end.

SEQ ID NO: 95 is SEQ ID NO: 38 without the additional nucleotides on the 3′ end.

SEQ ID NO: 96 is SEQ ID NO: 61 without the predicted chloroplast targeting peptide

SEQ ID NO: 97 is the C. reinhardtii chloroplast genome codon-optimized nucleotide sequence of SEQ ID NO: 96 with an additional 5′ start codon to encode for a methionine.

SEQ ID NO: 98 is SEQ ID NO: 64 without the added 3′ sequence encoding for an affinity tag.

SEQ ID NO: 99 is SEQ ID NO: 65 without the additional N-terminal start codon methionine or the C-terminal affinity tag.

SEQ ID NO: 100 is SEQ ID NO: 67 without the added 3′ sequence encoding for an affinity tag.

### Culture Conditions

Algae can typically be grown on a simple defined medium with light as the sole energy source. In some instances, a couple of fluorescent light bulbs at a distance of 1-2 feet is adequate to supply energy for growth. Some algae useful in the methods disclosed herein can be grown on agar plates or in liquid media, for example. During growth in liquid media, bubbling with, for example, air or 5% CO₂, may improve the growth rate. If the lights are turned on and off at regular intervals (for example, 12:12 or 14:10 hours of light:dark) the cell division cycle of some algae can be synchronized.

The fundamental requirements for algal growth are light, CO₂ and water. Open systems such as ponds, lakes, channels, or large open tanks are vulnerable to being contaminated, particularly given the possibility that other organisms that may take advantage of the culture system may reproduce more quickly than the alga used for bioproduction, decontamination, or carbon fixation. Nevertheless, the cost benefits of this type of open system may be significant.

A host organism or algae, in some embodiments, is grown under conditions which permit photosynthesis, however, this is not a requirement (e.g., a host organism may be grown in the absence of light). In some instances, the host organism may be genetically modified in such a way that photosynthetic capability is diminished and/or destroyed. In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, or an organism-specific requirement. Organic carbon sources include any source of carbon which the host organism is able to metabolize including, but not limited to, acetate, simple carbohydrates (e.g., glucose, sucrose, or lactose), complex carbohydrates (e.g., starch or glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

A host organism or algae can be grown on land, e.g., ponds, aqueducts, landfills, or in closed or partially closed systems. The host organisms herein can also be grown directly in water, e.g., in ocean, sea, on lakes, rivers, or reservoirs. In embodiments where algae are mass-cultured, the algae can be grown in high density photobioreactors, for example. Methods of mass-culturing algae are known. For example, algae can be grown in high density photobioreactors (for example, as described in Lee et al, Biotech. Bioengineering 44:1161-1167, 1994) and other bioreactors (such as those for sewage and waste water treatments) (for example, as described in Sawayama et al, Appl. Micro. Biotech., 41:729-731, 1994). Additionally, algae may be mass-cultured for removal of, for example, heavy metals (for example, as described in Wilkinson, Biotech. Letters, 11:861-864, 1989), hydrogen (for example, as described in U.S. Patent Application Publication No. 20030162273), and pharmaceutical compounds, from a water, soil, or other source.

A semi-closed system, such as a covered pond or pool, or a pond or pool within a greenhouse-type structure, can also be used. While this usually results in a smaller system, it allows for greater control of environmental conditions, which can permit the use of more algal species, and can extend the growing season. It is also possible to increase the amount of CO₂ in these semi-closed systems, thus increasing the rate of growth of the algae. However, these types of systems are also at risk of having species other than the host algal species colonize the liquid environment.

A variation of the pond system is an artificial pond e.g., a raceway pond. In these ponds, the algae, water, and nutrients circulate around a “racetrack.” With paddlewheels providing the flow, algae are kept suspended in the water, and are circulated back to the surface at a regular frequency. Raceway ponds are usually kept shallow because the algae need to be exposed to sunlight, and sunlight can only penetrate the pond water to a limited depth. However, depth can be varied according to the wavelength(s) utilized by an organism. The ponds can be operated in a continuous manner, with CO₂ and nutrients being constantly fed to the ponds, while algae-containing water is removed at the other end.

Alternatively, algae may be grown in closed structures such as photobioreactors (bioreactors incorporating a light source), where the environment is under stricter control than in open ponds. Because these systems are closed, carbon dioxide, water, and in most cases other nutrients need to be introduced into the system. Such artificial ponds and photobioreactors are therefore also vulnerable to contamination, particularly where the ponds or photobioreactors are designed to be continually or frequently harvested.

Algae that are genetically engineered for herbicide resistance are disclosed herein for growth in cultures, particularly but not exclusively large scale cultures, where large scale cultures refers herein to growth of algal cultures in volumes of greater than about 6 liters, greater than about 10 liters, greater than about 20 liters, greater than about 50 liters, greater than about 100 liters, greater than about 200 liters, greater than about 1,000 liters, greater than about 10,000 liters, greater than about 50,000 liters, or greater than about 100,000 liters. Large scale growth can be growth of algal cultures in ponds or other containers, vessels, or areas, where the pond, container, vessel or area that contains the algal culture is for example, from about 10 square meters or more in area to about 500 square meters in area or greater.

Large scale cultures of algae bioengineered for herbicide resistance can be used for the production of biomolecules, which can be therapeutic, nutritional, commercial, or fuel products, or for fixation of CO₂, or for decontamination of compounds, mixtures, samples, or solutions. The herbicide resistant algae provided herein can be grown in the presence of one or more herbicides that can impede or prevent the growth of species other than the algal species used for bioproduction, decontamination, or CO₂ fixation. In certain embodiments of the disclosure, a host alga transformed with one or more genes that confers herbicide resistance is transformed with one or more additional genes that encodes an additional heterologous or homologous protein that is produced by the alga when it is grown in culture, in which the additional heterologous or homologous protein is a therapeutic, nutritional, commercial, or fuel product, or increases production or facilitates isolation of a therapeutic, nutritional, commercial, or fuel product.

### Herbicide Resistant Algae

Genetically engineered algae containing one or more recombinant nucleotides that encode one or more proteins that confer resistance to one or more herbicides are provided. A herbicide resistant alga as provided herein includes at least one recombinant polynucleotide that encodes a protein that confers herbicide resistance, and may be used in some embodiments to produce biomolecules that are endogenous or not endogenous to the algal host. In some embodiments, the genetically engineered herbicide resistant algae can be cultured for environmental remediation or CO₂ fixation. The algae are transformed with one or more recombinant homologous or heterologous polynucleotides that enable growth of the algae in the presence of at least one herbicide.

### Prokaryotic Herbicide Resistant Algae

Provided in some embodiments herein is a herbicide resistant prokaryotic alga transformed with a homologous or heterologous polynucleotide encoding a protein that confers resistance to a herbicide. In some embodiments, the alga is a species of cyanobacteria. For example, the alga can be a Synechococcus, Anacytis, Anabaena, Athrospira, Nostoc, Spirulina, or Fremyella species. The alga species can include a heterologous polynucleotide integrated into its genome, in which the heterologous polynucleotide encodes a protein that confers resistance to glyphosate, a sulfonylurea, an imidazolinone, a 1,2,4-triazol pyrimidine, phosphinothricin, aminotriazole amitrole, an isoxazolidinones, an isoxazole, a diketonitrile, a triketone, a pyrazolinate, norflurazon, a bipyridylium, a p-nitrodiphenylether, an oxadiazole, an N-phenyl imide, atrazine, a triazine, diuron, DCMU, chlorsulfuron, imazaquin, a phenol herbicide, a halogenated hydrobenzonitrile, a urea herbicide, an aryloxyphenoxy propionate, a cyclohexandione oxime, a carotenoid biosynthesis inhibiting enzyme, or any combination of any two or more heterologous polypeptides. The herbicide resistance conferring protein can be, for example, 5-enolpyruvylshikimate-3-phosphate synthase (EPSPS), glyphosate oxidoreductase (GOX), glyphosate acetyl transferase (GAT), glutathione reductase, superoxide dismutase (SOD), acetolactate synthase (ALS), acetohydroxy acid synthase (AHAS), hydroxyphenylpyruvate dioxygenase (HPPD), bromoxynil nitrilase, hydroxyphenylpyruvate dioxygenase (HPPD), isoprenyl pyrophosphate isomerase, prenyl transferase, lycopene cyclase, phytoene desaturase, acetyl CoA carboxylase (ACCase) (or a subunit thereof), or cytochrome P450-NADH-cytochrome P450 oxidoreductase, where the encoded protein conferring herbicide resistance is not a cyanobacterial host species protein. In some embodiments, the heterologous polynucleotide encodes a protein conferring herbicide resistance. In some embodiments, the heterologous polynucleotide encodes 5-enolpyruvylshikimate-3-phosphate synthase (EPSPS), which can be a Class I or Class II EPSPS, or can be an EPSPS that does not belong to either Class I or Class II.

In some embodiments, a prokaryotic alga provided herein is resistant to two or more herbicides. A prokaryotic alga can include a first recombinant homologous or heterologous herbicide resistance gene conferring resistance to a first herbicide and a second herbicide resistance gene conferring resistance to a second herbicide. The second herbicide resistance gene may be endogenous to the alga, or may also be a recombinant homologous or heterologous herbicide resistance gene. Recombinant homologous resistance genes may in some embodiments be mutant forms of a homologous resistance gene.

The polynucleotide encoding the herbicide resistance gene can be provided in a vector for transformation of the algal host. In some embodiments, the vector is designed for integration into the host genome, and can include, for example, sequences having homology to the host genome flanking the herbicide resistance gene to promote homologous recombination. In other embodiments, the vector can have an origin of replication such that it can be maintained in the host as an autonomously replicating episome. In some embodiments, the protein-encoding sequence of the polynucleotide is codon biased to reflect the codon bias of the host alga.

### Eukaryotic Herbicide Resistant Algae

In some embodiments, the host alga transformed with a herbicide resistance gene is a eukaryotic alga. The host alga can be a macroalga or a microalga, and in some embodiments is a species of the Chlorophyta, and in some embodiments, the alga is a microalga, for example, a Chlamydomonas, Volvacales, Dunaliella, Scenedesmus, Chlorella, or Hematococcus species. A recombinant polynucleotide conferring herbicide resistance can be integrated into the nuclear genome or chloroplast genome of a eukaryotic host alga.

When the recombinant polynucleotide conferring the herbicide resistance is integrated into the chloroplast genome., but the encoded herbicide resistance gene is not, in its native state, a chloroplast-encoded gene, the sequence encoding the heterologous herbicide resistance protein, or encoding a homologous herbicide resistance protein that is a nuclear encoded protein, is in some embodiments synthesized with the codon bias of the host alga chloroplast genome to optimize expression in the chloroplast of the host alga. In these embodiments, a polynucleotide encoding a herbicide resistance protein can be operably linked to a chloroplast promoter, such as, for example, a 16SrRNA promoter, an rbcL promoter, an atpA promoter, a psaA promoter, a psbA promoter, or a psbD promoter. The herbicide resistance encoding polynucleotide, in some embodiments, is also operably linked to a 5′ UTR and, in some embodiments, a 3′ UTR that function in the chloroplast of the alga. The 5′UTR and 3′UTR can be from chloroplast-encoded genes, such as, but not limited to, rbcL, atpA, psaA, psbA, or psbD.

When the recombinant polynucleotide is integrated into the nuclear genome, but is not, in its native state, a gene encoded by the nuclear genome of the host algal species, the sequence encoding the heterologous herbicide resistance protein, is in some embodiments, synthesized with the codon bias of the host alga nuclear genome to optimize expression in the host alga. In these embodiments, a polynucleotide encoding a herbicide resistance protein can be operably linked to a promoter that is active in the host algal nucleus. A nuclear algal promoter used in constructs for expressing herbicide resistance genes in algae can be any nuclear algal promoter. Non-limiting examples of useful promoters are an RBCS (small subunit of ribulose bisphosphate carboxylase) promoter, an LHCP (light harvesting chlorophyll binding protein) promoter, a NIT 1 (nitrate reductase) promoter, a chimeric promoter, or a at least partially synthetic promoter. Any of these exemplary promoters can be used to express a herbicide resistance gene integrated into the nucleus of an alga. The herbicide resistance encoding polynucleotide in some embodiments is also operably linked to a 5′ UTR and a 3′ UTR that functions in the nucleus of the alga. In embodiments wherein the herbicide resistance gene does not include a sequence encoding a chloroplast transit peptide, but the polynucleotide encodes a protein that functions in the chloroplast of a eukaryotic alga, the polynucleotide can also include a transit peptide sequence that mediates import of the protein into the chloroplast. A chloroplast transit peptide sequence can be derived from any nuclear-encoded chloroplast protein, such as, for example, the RCBS precursor protein.

In one example, a glyphosate resistant eukaryotic alga contains a polynucleotide that encodes a homologous mutant 5-enolpyruvylshikimate-3-phosphate synthase (EPSPS) integrated into the chloroplast genome, in which the homologous mutant EPSP synthase confers glyphosate resistance. In this embodiment, the wild-type homologous EPSPS gene is homologous to the host species, although encoded in the nuclear genome. A cDNA sequence can be used for mutation of one or more codons of the EPSP gene to a glyphosate resistant form. In one embodiment, the codon corresponding to amino acid position 96 of the E. coli EPSP synthase (Genbank Accession No. A7ZYL1; GI: 166988249) (SEQ ID NO: 69), is mutated to encode alanine. In another embodiment, the codon corresponding to amino acid position 183 of the E. coli EPSP synthase (Genbank Accession No. A7ZYL1, GI: 166988249), is mutated to encode threonine. In some embodiments, both of the codons corresponding to codon 96 and codon 183 of the E. coli EPSP synthase (Genbank Accession No. A7ZYL1; GI: 166988249) are mutated to alanine and threonine, respectively.

In another instance, provided herein, is a herbicide resistant eukaryotic microalga containing a heterologous polynucleotide integrated into the chloroplast genome, in which the heterologous polynucleotide comprises a sequence that encodes glyphosate oxidoreductase (GOX), glyphosate acetyl transferase (GAT), or an EPSP synthase that is not a Class I EPSP synthase (for example, a Class II, or non-Class I/Class II EPSP synthase). The GOX, GAT, or non-Class I EPSP synthase gene is in some embodiments synthesized as a codon-biased gene whose nucleotide sequence conforms to the codon bias of the host algal chloroplast genome.

In another instance, provided herein is a herbicide resistant eukaryotic alga comprising a heterologous polynucleotide integrated into the chloroplast genome, in which the heterologous polynucleotide encodes a protein whose wild-type form is not encoded by the chloroplast genome, in which the protein confers resistance to a herbicide that does not inhibit amino acid synthesis. As nonlimiting examples, the heterologous polynucleotide can encode a protein conferring resistance to herbicides that inhibit carotenoid synthesis, inhibit fatty acid biosynthesis, inhibit photosynthesis, or cause photobleaching. The heterologous polynucleotide can encode a protein conferring resistance to, for example, an aminotriazole or aminotriazole amitrole, an isoxazolidinone, an isoxazole, a diketonitrile, a triketone, an aryloxyphenoxy propionate, a cyclohexandione oxime, a pyrazolinate, norflurazon, a bipyridylium, a p-nitrodiphenylether, an oxadiazole, an N-phenyl imide, or a halogenated hydrobenzonitrile herbicide. The heterologous polynucleotide can encode for example, glutathione reductase, superoxide dismutase (SOD), bromoxynil nitrilase, hydroxyphenylpyruvate dioxygenase (HPPD), isoprenyl pyrophosphate isomerase, prenyl transferase, lycopene cyclase., phytoene desaturase, actetyl CoA carboxylase (ACCase) (or subunits thereof), or cytochrome P450-NADH-cytochrome P450 oxidoreductase.

In a further instance, provided herein is a herbicide-resistant non-chlorophyll c-containing eukaryotic alga comprising a heterologous polynucleotide integrated into the nuclear genome, in which the heterologous polynucleotide encodes a protein that confers resistance to a herbicide, in which resistance to the herbicide is conferred by a single heterologous protein. The heterologous polynucleotide is in some embodiments operably linked to a heterologous promoter that functions in the nucleus of the host alga. The heterologous polynucleotide is in some embodiments provided with sequences homologous to the non-chlorophyll c-containing eukaryotic alga to promote recombination into the algal genome. In some embodiments, the polynucleotide encodes a protein that confers resistance to a non-antibiotic herbicide. A non-antibiotic herbicide is a herbicide that is not made by a microorganism, or whose chemical structure is not based on that of a compound made by a microorganism.

In some embodiments, the heterologous polynucleotide integrated into the genome of the non-chlorophyll c-containing eukaryotic alga encodes a 5-enolpyruvylshikimate-3-phosphate synthase (EPSPS), glyphosate oxidoreductase (GOX), glyphosate acetyl transferase (GAT), phosphinothricin acteyl transferase (PAT), glutathione reductase, superoxide dismutase (SOD), acetolactate synthase (ALS), acetohydroxy acid synthase (AHAS), hydroxyphenylpyruvate dioxygenase (HPPD), bromoxynil nitrilase, hydroxyphenylpyruvate dioxygenase (HPPD), isoprenyl pyrophosphate isomerase, prenyl transferase, lycopene cyclase, phytoene desaturase, actetyl CoA carboxylase (ACCase), or cytochrome P450-NADH-cytochrome P450 oxidoreductase. For example, the protein encoded by the heterologous polynucleotide in some embodiments confers resistance to glyphosate, and in some embodiments encodes a 5-enolpyruvylshikimate-3-phosphate synthase (EPSPS), a glyphosate oxidoreductase (GOX), or a glyphosate acetyl transferase (GAT). In some embodiments, the heterologous polynucleotide encodes a 5-enolpyruvylshikimate-3-phosphate synthase (EPSPS), which can be a Class I EPSPS, a Class II EPSPS, or a non Class I/Class II EPSPS.

Also provided herein, is a herbicide-resistant non-chlorophyll c-containing eukaryotic alga comprising a recombinant polynucleotide integrated into the nuclear genome, in which the recombinant polynucleotide encodes a homologous EPSPS protein that confers resistance to glyphosate. In some embodiments, the polynucleotide encodes a mutant homologous EPSP. In some embodiments, the host alga's endogenous EPSPS gene or cDNA is obtained or reconstructed by cloning of genomic DNA. Site-directed mutagenesis can be performed to introduce one or more particular mutations. Alternatively, PCR with primer(s) that contain the mutation(s) can be performed to create mutant genes. The entire gene or a portion of a gene can also be synthesized to include one or more mutations by using a set of overlapping primers, one or more of which include a mutation or mutations.

Also disclosed herein, is an isolated polynucleotide for transformation of a non-chlorophyll c-containing alga to herbicide resistance, wherein the polynucleotide encodes a heterologous protein that confers resistance to a herbicide, wherein the protein-encoding sequence is codon biased according to the codon bias of the nuclear genome of the alga. In some embodiments, the protein encoding sequence is codon biased to conform to the codon bias of the Chlamydomonas reinhardtii nuclear genome. The isolated polynucleotide, in some embodiments, includes a promoter that is active in the nuclear genome of the alga, for example, a rbcS promoter, an LHCP promoter, or a nitrate reductase promoter. The promoter can also be a chimeric promoter or a synthetic or partially synthetic promoter. For example, the isolated polynucleotide may have a naturally-occurring promoter sequence or may have additional sequences from another source to enhance transcription. In one example, a promoter that is active in the nuclear genome of C. reinhardtii has added sequences from the hsp 70A promoter (for example, as described in Lodha et al. Eukaryotic Cell 7: 172-176 (2008)). A nucleic acid construct that includes a codon biased sequence encoding a protein conferring herbicide resistance can also include a heterologous intron inserted into the protein encoding sequence. One example of an intron that can be inserted into a protein encoding sequence to enhance expression is an RBCS intron (for example, as described in Lumbreras et al. Plant J. 14: 441-447 (1998)). In some embodiments, the protein encoding sequence of the isolated polynucleotide further includes a chloroplast transit peptide-encoding sequence fused to the herbicide resistance protein encoding sequence.

Also provided herein, is an alga that includes a recombinant polynucleotide that encodes a Bacillus thuringiensis (Bt) toxin protein. In one embodiment, the alga includes a cry gene encoding the Bt toxin. The heterologous Bt toxin gene can be incorporated into the nucleus or the chloroplast of the alga. The alga can further include one or more recombinant nucleotides that encode a protein conferring resistance to a herbicide. An alga that is transformed with a recombinant polynucleotide encoding a Bt toxin protein can be a prokaryotic or a eukaryotic alga. In some embodiments, the alga is a cyanobacteria species. A recombinant polynucleotide encoding a Bt toxin gene is, in some embodiments, integrated into the genome of a prokaryotic host alga.

In some embodiments, the host alga transformed with a Bt toxin gene is a eukaryotic alga. In other embodiments, the host alga is a species of the Chlorophyta. In some embodiments, the alga is a microalga. A recombinant polynucleotide conferring herbicide resistance can be integrated into the nuclear genome or chloroplast genome of a eukaryotic host alga.

In some embodiments, an alga that has a gene encoding Bt toxin also has a recombinant polynucleotide encoding a protein that confers resistance to a herbicide.

In other embodiments a herbicide-resistant eukaryotic alga comprises two or more recombinant polynucleotide sequences encoding proteins that confer resistance to herbicides, in which each of the proteins confers resistance to a different herbicide. In some embodiments, a herbicide resistant alga transformed with herbicide resistance genes is resistant to two or more herbicides that inhibit different amino acid biosynthesis pathways, for example, glyphosate and sulfonylureas, or glyphosate and phosphinothricin. In some embodiments, a herbicide resistant alga transformed with herbicide resistance genes is resistant to two or more herbicides, in which at least one herbicide inhibits an amino acid biosynthesis pathway, and at least one herbicide does not inhibit an amino acid biosynthesis pathway. For example, a herbicide resistant alga can include recombinant genes conferring glyphosate resistance and resistance to norflurazon.

In some embodiments, at least one of the recombinant polynucleotides encoding a protein conferring herbicide resistance is integrated into the chloroplast genome of a eukaryotic alga. In some embodiments, at least one of the recombinant polynucleotides encoding a protein conferring herbicide resistance is integrated into the nuclear genome of a eukaryotic alga. In some embodiments, at least one of the two or more recombinant polynucleotides encoding a protein conferring herbicide resistance is integrated into the chloroplast genome and at least one of the two or more polynucleotide sequences encoding a protein conferring herbicide resistance is integrated into the nuclear genome of a eukaryotic alga. A polynucleotide encoding a herbicide resistance protein that is integrated into the chloroplast genome, in some instances, is codon biased to reflect the codon bias of the chloroplast genome of the host alga. A polynucleotide encoding a herbicide resistance protein that is integrated into the nuclear genome, in some instances, is codon biased to reflect the codon bias of the nuclear genome of the host alga.

In some embodiments of an alga comprising two or more recombinant polynucleotide sequences encoding proteins that confer resistance to herbicides, at least one of the recombinant polynucleotides encodes a homologous protein conferring herbicide resistance. In some embodiments, at least one of the polynucleotides encodes a heterologous protein conferring herbicide resistance.

In some embodiments, the herbicide resistant alga that has two different recombinant herbicide resistance genes is a microalga. In some embodiments, the alga that includes two different herbicide resistance genes is a prokaryotic alga, such as a cyanobacterial species. In some embodiments, the alga that includes two different herbicide resistance genes is a eukaryotic microalga, such as a Chlamydomonas, Volvacales, Dunaliella, Scenedesmus, Chlorella, or Hematococcus species. In another embodiment, the herbicide resistant alga that has two different recombinant herbicide resistance genes is a Chlamydomonas species.

Also provided herein, is a non chlorophyll c-containing herbicide-resistant alga comprising a recombinant polynucleotide encoding a protein that confers resistance to a herbicide and a heterologous polynucleotide encoding a protein that does not confer resistance to a herbicide, wherein the protein that does not confer resistance to a herbicide is an industrial enzyme or therapeutic protein, or a protein that participates in or promotes the synthesis of at least one nutritional, therapeutic, commercial, or fuel product, or a protein that facilitates the isolation of at least one nutritional, therapeutic, commercial, or fuel product. A nutritional product may be, as nonlimiting examples, a lipid, carotenoid, fatty acid, vitamin, cofactor, nucleotide, amino acid, peptide, or protein. A therapeutic product can be, for example, a vitamin, cofactor, amino acid, peptide, hormone, or growth factor. A therapeutic protein can be an antibody, hormone, growth factor, or clotting factor, for example. A commercial product can be a lubricant, insecticide, perfume, pigment, coloring agent, flavoring agent, enzyme, adhesive, thickener, solubilizer, stabilizer, surfactant, or coating, for example. A fuel product can be, without limitation, any of a lipid, a fatty acid, a hydrocarbon, a carbohydrate, cellulose, glycerol, an alcohol, or any combination of the above. An industrial enzyme can be, for example, a beta-glucosidase, a xylanase, an endoglucanase, a cellobiohydrolase, an alpha-amylase, a lipase, a phospholipase A1, a phospholipase C, or a protease.

Also disclosed herein, are methods of producing one or more biomolecules, in which the methods include transforming an alga with a polynucleotide encoding Bt toxin protein, growing the alga under conditions in which the Bt toxin is expressed, and harvesting one or more biomolecules from the alga or algal media. The methods, in some embodiments, include isolating the one or more biomolecules.

Also disclosed herein, are methods of producing one or more biomolecules, in which the methods include transforming an alga with a polynucleotide encoding a protein conferring herbicide resistance, growing the alga in the presence of the herbicide, and harvesting one or more biomolecules from the alga or algal media. The methods, in some embodiments, include isolating the one or more biomolecules.

The genetically engineered herbicide resistant alga is grown in media containing a concentration of herbicide that permits growth of the transformed alga, but inhibits growth of the same species of alga that is not transformed with a gene encoding a protein that confers resistance to the herbicide. In some embodiments, the concentration of herbicide in the media in which the genetically engineered alga is grown to produce a biomolecule or product, inhibits the growth of at least one other algal species. In some embodiments, the concentration of herbicide in the media in which the genetically engineered alga is grown to produce a biomolecule or product, inhibits the growth of at least one bacterial species or at least one fungal species. The concentration for optimal bioproduction by the host alga and inhibition of growth of other nontransformed species can be empirically determined, and can be, for example, in the sub-micromolar to millimolar range.

In some embodiments, genetically engineered herbicide resistant algae that include two or more recombinant polynucleotides encoding proteins each conferring resistance to a different herbicide are grown in media containing two or more herbicides. The two or more herbicides in combination can inhibit the growth of any combination of at least one algal species, at least one bacterial species, and/or at least one fungal species.

A product (for example, fuel products, fragrance products, insecticide products, commercial products, and therapeutic products) may be produced by an algal culture by a method that comprises the step of: growing/culturing a herbicide resistant alga transformed by one or more of the herbicide resistance-conferring nucleic acids described herein in media that includes at least one herbicide. In some instances, the media includes glyphosate. In some instances, the media includes imidazoline. The methods herein can further comprise the step of collecting the product produced by the organism or algae. The product can be the product of a heterologous nucleotide also transformed into the alga.

In some embodiments, the product (for example, fuel products, fragrance products, or insecticide products) is collected by harvesting the algae. The product may then be extracted from the algae.

In one embodiment, methods are provided for producing a biomass-degrading enzyme in an alga, in which the methods include transforming the alga with a polynucleotide comprising a sequence conferring herbicide tolerance to the alga and a sequence encoding an exogenous biomass-degrading enzyme or a sequence encoding a protein or a nucleotide sequence which promotes increased expression of an endogenous biomass-degrading enzyme, growing the alga in the presence of the herbicide and under conditions which allow for production of the biomass-degrading enzyme, in which the herbicide is in sufficient concentration to inhibit growth of the alga which does not include the sequence conferring herbicide tolerance, to producing the biomass-degrading enzyme. The methods in some embodiments include isolating the biomass-degrading enzyme. Exemplary biomass-degrading enzymes, that may be used in the methods described herein, are described in International Patent Application No. PCT/US2008/006879, filed May 30, 2008. In one embodiment, the biomass-degrading enzyme is chlorophyllase.

A sufficient concentration of herbicide is an amount such that the algae that is not transformed is killed or the growth of the untransformed algae is substantially inhibited in comparison to the transformed algae. One of skill in the art would be able to determine the proper concentration of herbicide to use without undue experimentation.

Provided below is an exemplary chart of herbicide concentrations that can be used in the embodiments disclosed herein. The concentrations provided are the concentration that growth of the wild type algae is inhibited at, and the highest concentrations that an isolated resistant strain of Chlamydomonas reinhardtii can tolerate. One of skill in the art would be able to determine the proper concentration of the herbicides listed in the chart without undue experimentation.

| | DCMU (3-(3,4- | | | | | | | |
|---|---|---|---|---|---|---|---|---|
| | dichlorophenyl)- | | | | | | | |
| | 1.1-dimethylurea) | Atrazine | Bromacil | Glyphosate | Chlorsulfuron | Imazaquin | Norflurazon | Paraqual |
| Wildtype | 2 μM | 5 μM | 2 μM | 1 mM | 0.1 mM | 1.0 mM | 1.1 μm | 0.7 μM |
| Chlamydomonas | | | | | | | | |
| reinhardiii | | | | | | | | |
| Resistant | 200 μM | 100 μM | 50 μM | 5 mM | 1 mM | 10 mM | 3.6 μM | 54 μM |
| Chlamydomonas | | | | | | | | |
| reinhardiii | | | | | | | | |
| | Complete Growth inhibition | Complete | Complete | Complete | Complete | Complete | I₅₀ | I₅₀ |
| | | Growth | Growth | Growth | Growth | Growth | | |
| | | inhibition | inhibition | inhibition | inhibition | inhibition | | |
| | Galloway RE and | Galloway | Galloway | Unpublished | Winder T and | Winder T | Vartak Vand | Vartak |
| | Mets LJ, Plant | RE and | RE and | results | Spalding MH, | and | Sujata B, | Vand |
| | Physiol 74; 469-474: 1984 | Mets LJ, | Mets LJ, | | Mol Gen | Spalding | Weed Sci | Sujata B, |
| | | Plant | Plant | | Genetics | MH, Mol | 45; 374-377: | Pesticide |
| | | Physiol | Physiol | | 213; 394-399: | Gem | 1997 | Biochem |
| | | 74; 469-474: | 74; 469-474: | | 1988. | Genetics | | Physiol |
| | | 1984 | 1984 | | | 213; 394-399: | | 64; 9-15: |
| | | | | | | 1988 | | 1999 |

In some embodiments, the expression of the product (for example fuel product, fragrance product, or insecticide product) is inducible. The product may be induced to be expressed. Expression may be inducible by light. In yet other embodiments, the production of the product is autoregulatable. The product may form a feedback loop, for example, wherein when the product (for example fuel product, fragrance product, or insecticide product) reaches a certain level, expression of the product may be inhibited by the product itself. In other embodiments, the level of a metabolite present in the algae inhibits expression of the product. For example, endogenous ATP produced by the algae as a result of increased energy production to express the product, may form a feedback loop to inhibit expression of the product. In yet another embodiment, production of the product may be inducible, for example, by light or an exogenous agent. For example, an expression vector for effecting production of a product in the host algae may comprise an inducible regulatory control sequence that is activated or inactivated by an exogenous agent.

The methods herein may further comprise the step of providing to the organism or algae a source of inorganic carbons, such as flue gas. In some instances, the inorganic carbon source provides all of the carbon necessary for making the product (for example, fuel product). The growing/culturing step occurs in a suitable medium, such as one that has minerals and/or vitamins in addition to at least one herbicide.

The methods described herein include, but are not limited to, selecting genes that are useful to produce products, such as fuels, fragrances, therapeutic compounds, or insecticides, transforming genetically engineered herbicide resistant algae with such gene(s), and growing such algae in the presence of at least one herbicide under conditions suitable to allow the product to be produced. Organisms such as algae can be cultured in conventional fermentation bioreactors, which include, but are not limited to, batch, fed-batch, cell recycle, and continuous fermentors. Further, they may be grown in photobioreactors (for example, as described in US Appl. Publ. No. 20050260553; U.S. Pat. No. 5,958,761; and U.S. Pat. No. 6,083,740). Culturing or growing of the algae can also be conducted in shake flasks, test tubes, microtiter dishes, and petri plates, for example. Culturing or growing can be carried out at a temperature, pH, and oxygen content appropriate for the recombinant algae, and at a herbicide concentration that permits growth and bioproduction by the host algae that have been transformed with herbicide resistance genes.

The transformed herbicide resistant algae and methods provided herein can expand the culturing conditions of the host algae to larger areas that may be open and, in the absence of herbicide resistance, subject to contamination of the culture, for example, on land, such as in landfills. In some cases, host organism(s) are grown near ethanol production plants or other facilities or regions (for example, cities, or highways) generating CO₂. As such, the methods herein contemplate business methods for selling carbon credits to ethanol plants or other facilities or regions generating CO₂ while making fuels by growing one or more of the modified organisms described herein in the presence of a herbicide.

Further, the organisms may be grown, for example, in outdoor open water, such as ponds, waterbeds, shallow pools, reservoirs, tanks, or canals, to which herbicide can be added to repress growth of any of bacteria, fungi, and/or nontransformed algal species.

The following examples are intended to provide illustrations of the application of the present disclosure. The following examples are not intended to completely define or otherwise limit the scope of the disclosure.

### EXAMPLES

### Example 1

This examples describes the construction of exemplary nucleic acid constructs that can be used in the methods disclosed herein.

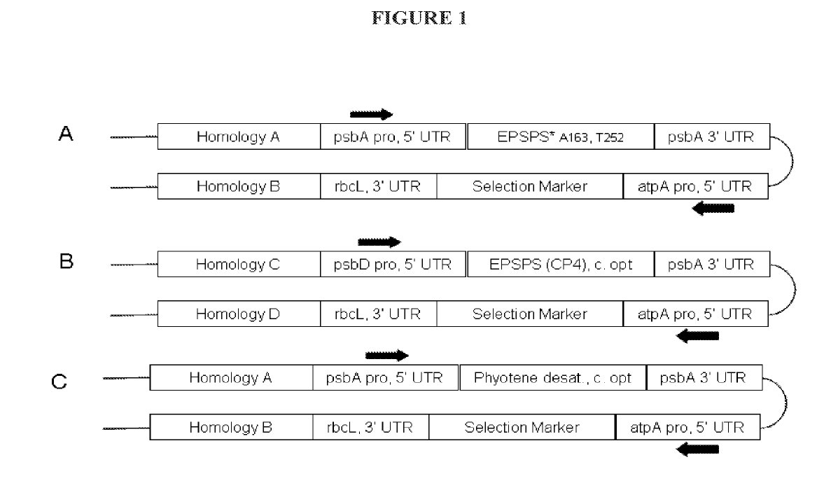

The constructs depicted in FIG. 1 can further include an origin of replication for producing the construct in bacteria or yeast, and an additional selectable marker for use in bacteria or yeast (not shown). A) is a schematic diagram of a portion of a construct that includes a mutant EPSPS gene conferring glyphosate resistance and a kanamycin resistance gene flanked by chloroplast genome homology regions, where each gene is operably linked to its own regulatory sequences. B) is a schematic diagram of a portion of a construct that includes a codon-biased gene encoding a Class II EPSP (“CP4”) that confers glyphosate resistance and a kanamycin resistance gene flanked by chloroplast genome homology regions, where each gene is operably linked to its own regulatory sequences. C) is a schematic diagram of a portion of a construct that includes a gene encoding a phytoene desaturase that confers resistance to norflurazon and a kanamycin resistance gene flanked by chloroplast genome homology regions, where each gene is operably linked to its own regulatory sequences.

### Example 2

This example describes the prokaryotic alga Synechocystis sp. Strain PCC6803 transformed with a gene conferring glyphosate resistance.

A construct that includes an EPSPS encoding nucleotide sequence of an unknown bacterium, sequence identifier number three of U.S. Pat. No. 7,238,508 (SEQ ID NO: 5), is operably linked to a promoter and terminator sequence active in Synechocystis. The construct also includes a selectable marker, the ampicillin resistance gene. The EPSPS gene is codon biased to reflect the codon bias of the Synechocystis genome. The EPSPS gene and regulatory sequences are flanked by sequences having homology to the Synechocystis genome for homologous recombination of the gene into the Synechocystis genome. The amino acid sequence of the EPSPS gene is shown in SEQ ID NO: 6. All DNA manipulations are carried out essentially as described by Sambrook et al., Molecular Cloning: A Laboratory Manual (Cold Spring Harbor Laboratory Press 1989) and Cohen et al., Meth. Enzymol. 297, 192-208, 1998.

For transformation with the herbicide resistance gene, Synechocystis sp. strain 6803 is grown to a density of approximately 2×10⁸ cells per ml and harvested by centrifugation. The cell pellet is re-suspended in fresh BG-1 medium (ATCC Medium 616) at a density of 1×10⁹ cells per ml and used immediately for transformation. One-hundred microliters of these cells are mixed with 5 ul of a mini-prep solution containing the construct and the cells are incubated with light at 30° C. for 4 hours. This mixture is then plated onto nylon filters resting on BG-11 agar supplemented with TES pH 8.0 and grown for 12-18 hours. The filters are then transferred to BG-11 agar+TES+5 ug/ml ampicillin and allowed to grow until colonies appear, typically within 7-10 days.

Colonies are then picked into BG-11 liquid media containing 5 μg/ml ampicillin and grown for 5 days. The transformed cells are incubated under low light intensity for 1-2 days and thereafter moved to normal growth conditions. These cells are then transferred to BG-11 media containing 10 μg/ml ampicillin and allowed to grow for typically 5 days. Cells are then harvested for PCR analysis to determine the presence of the exogenous insert. Western blots may be performed to determine expression levels of the protein(s) encoded by the inserted construct.

### Example 3

This example demonstrates transformation of an algal chloroplast with a gene encoding homologous EPSP synthase, mutated to a form that confers resistance to glyphosate, to provide a glyphosate resistant alga.

The amino acid sequence of 5-enolpyruvylshikimate-3-phosphate synthase (EPSPS) of Chlamydomonas reinhardtii (Genbank Accession number XP_{—}001702942, GI: 159489926 (SEQ ID NO: 1)) is modified such that the glycine residue at position 163 of the precursor protein (the form that includes the transit peptide) is changed to alanine and the alanine residue at position 252 is changed to threonine (SEQ ID NO: 2). These amino acid positions correspond to positions 101 and 192 of the amino acid sequence of the predicted mature EPSPS protein (based on analogy of the C. reinhardtii EPSPS sequence to that of other mature EPSP sequences (for example, as shown in sequence identifier number one of U.S. Pat. No. 6,225,114) (SEQ ID NO: 7). The sequence of the mature C. reinhardtii EPSPS is obtained using homology with plant EPSPS protein sequences and the predicted cleavage site for chloroplast transit peptides identified using a program for predicting transit peptides and their cleavage sites (ChloroP, available at the URL link cbs.dur.dk/services/ChloroP/; and Emanuelsson, O. et al., Protein Science. 8:978-984 (1999)) and is converted to DNA sequence, in which the codon usage reflects the chloroplast genome codon bias of Chlamydomonas reinhardtii (for example, as described in Franklin et al. Plant J. 30: 733-744 (2002); Mayfield et al. Proc. Natl Acad Sci. USA 100: 438-442 (2003); and U.S. Patent Application Publication No. 2004/0014174). The codon-optimized sequence is used to synthesize a codon-optimized mature C. reinhardtii EPSPS coding sequence according to the oligo assembly method of Stemmer et al. (for example, as described in Gene 164: 49-53 (1995)). It is understood that PCR conditions can be modified with regard to, for example, reagent concentrations, temperatures, duration of each step, and cycle number, to optimize production of the desired polynucleotide.

Approximately 65 oligonucleotides are synthesized to span the approximately 1,335 bp nucleotide sequence encoding the mature codon optimized and doubly mutated C. reinhardtii EPSPS gene. The oligos are designed to incorporate optimized C. reinhardtii chloroplast codons and mutated amino acid codons. The oligos are 40 nucleotides in length, and comprise sequences from both strands of the gene, such that the oligos from opposite strands overlap one another and hybridize to one another in the regions of overlap. In the gene assembly PCR reactions, regions where there is no overlap (for example, regions that are single-stranded when the full set of oligos is hybridized) are filled-in by a polymerase. The outermost (5′most) oligos from each strand incorporate unique restriction sites for further cloning. The gene assembly PCR step is performed for 30-65 cycles, with the conditions optimized for production of a 1.335 kb full-length gene product. In one instance, PCR reactions for gene assembly are performed using 0.2 micromolar of each oligo in a reaction mix containing 10 mM Tris-HCl, pH 9.0, 0.1% Triton X-100, 2.2 mM MgCl₂, 50 mM KCl, 0.2 mM each of dATP, dCTP, dGTP, and dTTP, and 1 unit of Taq polymerase. Thirty cycles are performed of 30 seconds at 94 degrees C., 30 seconds at 52 degrees C., and 30 seconds at 72 degrees C.

The gene assembly PCR product is confirmed by gel electrophoresis of an aliquot of the PCR reaction, and then the gene assembly PCR reaction is diluted 40-fold into a 100 microliter PCR reaction that includes the two outermost primers (the 5′ most primers of either strand) at 1 micromolar each, 10 mM Tris-HCl, pH 9.0, 0.1% Triton X-100, 2.2 mM MgCl₂, 50 mM KCl, 0.2 mM each of dATP, dCTP, dGTP, and dTTP, and 1 unit of Taq polymerase. For gene amplification, 20 cycles are performed of 30 seconds at 94 degrees C., 30 seconds at 50 degrees C., and 70 seconds at 72 degrees C. Following the amplification reactions, the PCR product is purified by phenol and chloroform extraction, ethanol precipitated, and digested with the enzymes recognizing the unique restriction sites at either end of the gene amplification product.

The digest is electrophoresed and the digested gene product is gel-purified prior to cloning the codon-optimized, double mutated EPSPS gene into the chloroplast cloning vector, depicted in FIG. 1A and described in Example 1, that includes the 5′ UTR and promoter sequence for the psbA gene from C. reinhardtii and the 3′ UTR for the psbA gene from C. reinhardtii. A kanamycin resistance gene from bacteria is used as the “Selection Marker”, which is regulated by the 5′ UTR and promoter sequence for the atpA gene from C. reinhardtii and the 3′ UTR sequence for the rbcL gene from C. reinhardtii. The transgene cassette is targeted to the psbA loci of the C. reinhardtii chloroplast genome via the segments labeled “Homology A” and “Homology B,” which are identical to sequences of DNA flanking the psbA locus on the 5′ and 3′ sides of the psbA gene, respectively, in the inverted repeat of the chloroplast genome (for example, as described in Maul et al. The Plant Cell 14: 2659-2679; also available at the URL link: “biology.duke.edu/chlamy_genome/-chloro.htmnl”). All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297: 192-208, 1998.

All transformations are carried out on C. reinhardtii strain 137c (mt+). Cells are grown to late log phase (approximately 7 days) in the presence of 0.5 mM 5-fluorodeoxyuridine in TAP medium (for example, as described in Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty mls of cells are harvested by centrifugation at 4,000×g at 23° C. for 5 min. The supernatant is decanted and cells are resuspended in 4 ml TAP medium and spread on TAP plates that include (for example, 100 g/ml) kanamycin or glyphosate, for subsequent chloroplast transformation by particle bombardment (for example, as described in Cohen et al., Meth. Enzymol. 297: 192-208, 1998). Exemplary concentrations of glyphosate range from about 1 mM to about 6 mM. For example, a concentration of 5.5 mM glyphosate can be used.

Following particle bombardment the number of transformants recovered from each type of selection is compared. Cells selected on kanamycin or glyphosate are replica plated on TAP plates that include different concentrations of glyphosate to determine the level of glyphosate resistance in kanamycin selected cells.

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algal lysates in EDTA are added to provide template for the reactions. Magnesium concentration is varied to compensate for amount and concentration of algae lysate in EDTA added. Annealing temperature gradients are employed to determine optimal annealing temperature for specific primer pairs.

To identify strains that contain the EPSPS gene, a primer pair is used in which one primer anneals to a site within the psbA 5′UTR and the other primer anneals within the EPSPS coding segment. Desired clones are those that yield a PCR product of the expected size for the psbA 5′UTR linked to the recombinant EPSPS gene. To determine the degree to which the endogenous gene locus is displaced (heteroplasmic vs. homoplasmic), a PCR reaction consisting of two sets of primer pairs in the same reaction is employed. The first pair of primers amplifies the endogenous locus targeted by the expression vector and consists of a first primer that anneals within the psbA 5′UTR and a second primer that anneals within the psbA coding region. This primer pair only amplifies the psbA region of a chloroplast genome in which the EPSP gene construct has not been integrated. The second pair of primers amplifies a constant, or control, region that is not targeted by the expression vector, and should produce a product of expected size whether or not the recombinant resistance gene is integrated into the chloroplast genome. This reaction is to confirm that the absence of a PCR product from the endogenous locus does not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is >30 to increase sensitivity. The most desired clones are those that yield a product for the constant region but not for the endogenous gene locus. Desired clones are also those that give weak-intensity endogenous locus products relative to the control reaction.

### Example 4

This example provides an alga having a heterologous EPSP synthase that confers resistance to glyphosate, integrated into the chloroplast genome.

The amino acid sequence of the EPSPS gene of Agrobacterium tumafaciens strain CP4 (Genbank Accession number Q9R4E4, GI: 8469107 (SEQ ID NO: 3)) is converted to a codon-optimized DNA sequence (SEQ ID NO: 56), in which the codon usage reflects the chloroplast codon bias of Chlamydomonas reinhardtii (Franklin et al. Plant J. 30: 733-744 (2002); Mayfield et al. Proc. Natl Acad Sci. USA 100: 438-442 (2003); see U.S. Patent Application Publication No. 2004/0014174). The codon-optimized CP4 EPSPS nucleotide sequence is used to synthesize a codon-optimized CP4 EPSPS gene according to the oligo assembly method of Stemmer et al. (Gene 164: 49-53 (1995)), as detailed above in Example 3 for the C. reinhardtii EPSPS gene.

The digested gene product is gel-purified prior to cloning the codon-optimized, CP4 gene into chloroplast cloning vector depicted in FIG. 1B that includes the 5′ UTR and promoter sequence for the psbD gene from C. reinhardtii and the 3′ UTR for the psbA gene from C. reinhardtii. The transgene cassette is targeted to the 3HB locus of C. reinhardtii via the segments labeled “Homology C” and “Homology D,” which are identical to sequences of DNA flanking the 31-HB locus on the 5′ and 3′ sides, respectively. All DNA manipulations are carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297: 192-208, 1998.

All transformations are carried out on C. reinhardtii strain cc1690 (mt+). Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells are resuspended in 4 ml TAP medium and spread on TAP plates that include (100 μg/ml) kanamycin, for subsequent chloroplast transformation by particle bombardment (Cohen et al., supra, 1998).

Following particle bombardment the number of transformants recovered from each type of selection is compared. Cells selected on glyphosate are replica plated on TAP plates that include different concentrations of glyphosate to determine the level of glyphosate resistance in selected cells.

PCR is used to identify transformed strains (see U.S. Patent Application Publication No. 2009/0253169). For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algal lysates in EDTA are added to provide template for the reactions. Magnesium concentration is varied to compensate for amount and concentration of algae lysate in EDTA added. Annealing temperature gradients are employed to determine optimal annealing temperature for specific primer pairs.

To identify strains that contain the codon-optimized CP4 Class II EPSPS gene, a primer pair is used in which one primer anneals to a site within the psbD 5′UTR and the other primer anneals within the CP4 EPSPS coding segment. Desired clones are those that yield a PCR product of the expected size for the psbD 5′UTR linked to the recombinant CP4 EPSPS gene. To determine the degree to which the endogenous gene locus is displaced (heteroplasmic vs. homoplasmic), a PCR reaction consisting of two sets of primer pairs in the same reaction is employed. The first pair of primers amplifies the endogenous locus targeted by the expression vector and consists of a first primer that anneals within the psbD 5′UTR and a second primer that anneals within the psbD coding region. This primer pair only amplifies the psbD region of a chloroplast genome in which the CP4 EPSP gene construct has not been integrated. The second pair of primers amplifies a constant, or control, region that is not targeted by the expression vector, and should produce a product of expected size whether or not the recombinant resistance gene is integrated into the chloroplast genome. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is greater than 30 to increase sensitivity. The most desired clones are those that yield a product for the constant region but not for the endogenous gene locus. Desired clones are also those that give weak-intensity endogenous locus products relative to the control reaction.

### Example 5

This example demonstrates transformation of an algal chloroplast with a gene encoding a heterologous phytoene desaturase to produce a norflurazon resistant alga.

The amino acid sequence of phytoene desaturase of a norflurazon resistant Synechococcus sp strain PCC 7942 (Genbank as Accession number CAA39004, GI: 48056 (SEQ ID NO: 4), is converted to DNA sequence, in which the codon usage reflects the codon bias of the chloroplast genome of Chlamydomonas reinhardtii (for example, as described in Franklin et al. Plant J. 30: 733-744 (2002); Mayfield et al. Proc. Natl. Acad Sci. USA 100: 438-442 (2003): and U.S. Patent Application Publication No. 2004/0014174). The codon-optimized sequence is used to synthesize a codon-optimized mature C. reinhardtii phytoene desaturase coding sequence according to the oligo assembly method of Stemmer et al. (Gene 164: 49-53 (1995)).

The digest is electrophoresed and the digested gene product is gel-purified prior to cloning the codon-optimized phytoene synthase gene into chloroplast cloning vector depicted in FIG. 1C that includes the 5′ UTR and promoter sequence for the psbA gene from C. reinhardtii and the 3′ UTR for the psbA gene from C. reinhardtii. A kanamycin resistance gene from bacteria is used as the “Selection Marker”, which is regulated by the 5′ UTR and promoter sequence for the atpA gene from C. reinhardtii and the 3′ UTR sequence for the rbcL gene from C. reinhardtii. The transgene cassette is targeted to the psbA loci of the C. reinhardtii chloroplast genome via the segments labeled “Homology A” and “Homology B,” which are identical to sequences of DNA flanking the psbA locus on the 5′ and 3′ sides of the psbA gene, respectively, in the inverted repeat of the chloroplast genome. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

All transformations are carried out on C. reinhardtii strain 137c (mt+). Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty mls of cells are harvested by centrifugation at 4,000×g at 23° C. for 5 min. The supernatant is decanted and cells are resuspended in 4 ml TAP medium for subsequent chloroplast transformation by particle bombardment (Cohen et al., Meth. Enzymol. 297: 192-208, 1998).

Following particle bombardment, some cells are selected on kanamycin selection (100 μg/ml) in which resistance is conferred by the kanamycin gene of the transformation vector (FIG. 1C). Other cells are selected on TAP plates that include to norflurazon. The number of transformants recovered from each type of selection is compared. Cells selected on kanamycin or glyphosate are replica plated on TAP plates that contain a range of concentrations of norflurazon to determine the level of norflurazon resistance in kanamycin selected cells.

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algal lysates in EDTA are added to provide template for the reactions. Magnesium concentration is varied to compensate for amount and concentration of algae lysate in EDTA added. Annealing temperature gradients are employed to determine optimal annealing temperature for specific primer pairs.

To identify strains that contain the phytoene desaturase gene, a primer pair is used in which one primer anneals to a site within the psbA 5′UTR and the other primer anneals within the phytoene desaturase coding segment. Desired clones are those that yield a PCR product of the expected size for the psbA 5′UTR linked to the recombinant phytoene desaturase gene. To determine the degree to which the endogenous gene locus is displaced (heteroplasmic vs. homoplasmic), a PCR reaction consisting of two sets of primer pairs in the same reaction is employed. The first pair of primers amplifies the endogenous locus targeted by the expression vector and consists of a first primer that anneals within the psbA 5′UTR and a second primer that anneals within the psbA coding region. This primer pair only amplifies the psbA region of a chloroplast genome in which the phytoene desaturase gene construct has not been integrated. The second pair of primers amplifies a constant, or control, region that is not targeted by the expression vector, and should produce a product of expected size whether or not the recombinant resistance gene is integrated into the chloroplast genome. This reaction confirms that the absence of a PCR product from the endogenous locus does not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is 30 or more to increase sensitivity. The most desired clones are those that yield a product for the constant region but not for the endogenous gene locus. Desired clones are also those that give weak-intensity endogenous locus products relative to the control reaction.

### Example 6

This example demonstrates transformation of an alga with a homologous gene encoding EPSP synthase that has been mutated to a form that confers resistance to glyphosate.

The nucleotide sequence of 5-enolpyruvylshikimate-3-phosphate synthase (EPSPS) of Chlamydomonas reinhardtii (Genbank as Accession number XM_{—}001702890, GI: 159489925 (SEQ ID NO:1)) is modified such that the codon encoding the glycine residue at position 163 of the precursor protein (the form that includes the transit peptide) is changed to an alanine codon, and the alanine codon at position 252 of the precursor protein is changed to a threonine codon (SEQ ID NO:2). These codons correspond to codons 101 and 192 of the mature EPSPS protein (based on analogy of the C. reinhardtii EPSPS sequence to that of sequence identifier number 1 of U.S. Pat. No. 6,225,114) (SEQ ID NO: 7). The mutations are introduced by PCR reactions using primers that incorporate the codon mutations, or by synthesis of a gene using the oligo assembly method of Stemmer et al. (Gene 164: 49-53 (1995)) outlined in the above examples, in which the oligos incorporate the mutated codon sequences. The coding regions and 3′ UTR of the mutant EPSPS gene is cloned 3′ to the promoter and 5′ UTR of the rbcS2 gene (for example, as described in Goldschmidt-Clermont and Rahirc, J. Mol. Bio. 191: 421-432 (1986); Kozminski et al. Cell Motil. Cytoskel. 25: 158-170; and Nelson et al. Mol. Cell. Biol. 14: 4011-4019 (1994), and inserted into a pUC-based plasmid that includes the hygromycin resistance gene, which confers resistance to hygromycin (Marsh, Gene 32:481-485, 1984).

For transformation by electroporation. C. reinhardtii cells are grown to approximately 1-5×10⁶ cells/ml or until the cells are in mid-log phase. A 1:2000 dilution of sterile 10% Tween-20 is added to the cells and the cells are centrifuged as gently as possible between 2000 and 5000 g for 5 min. The supernatant is removed and the cells are resuspended in TAP+60 mM sucrose media. The resuspended cells are placed on ice. To prepare the electroporation cuvettes, 5 ul of 10 mg/ml single stranded, sonicated, heat-denatured salmon sperm DNA is pipetted into a cuvette and then 2.5 ug of DNA is added to each cuvette. 250 ul of the cell suspension is added and the cuvettes are placed into a chamber that cools the cuvettes to 15° C. for 2 minutes. The electroporator capacitance is set at 3 μF and the voltage is set at 1.8 kV to deliver V/cm of 4500. The time constant is set for 1.2-1.4 ms. After delivering the pulse, the cuvette is returned to the 15° C. chamber. Cells are plated on plates that include hygromycin within an hour of electroporation by pipetting 1-1.5 ml of cornstarch solution onto a plate and then pipetting an aliquot of the electroporation mixture into the solution. To spread the cells and cornstarch, the plate is tilted slightly and rocked gently. The plates are allowed to dry in a sterile hood, and then placed in low light (5 μE) for twenty-four hours before moving them to growth conditions (80 μE).

Hygromycin-resistant colonies will be replica plated and grown in the presence of from 1 mg/liter to 5 g/liter glyphosate to test transformants for glyphosate resistance. PCR and/or Southern blot analysis with a probe for the EPSPS gene is used to confirm that resistant cells have integrated the transforming DNA.

### Example 7

This example provides a eukaryotic alga genetically engineered to have two recombinant polynucleotides that confer resistance to two herbicides.

A Chlamydomonas nuclear transformant of Example 6, transformed with a homologous mutant EPSPS gene that confers resistance to glyphosate, is used as a host cell for chloroplast transformation with the large and small subunit of the ALS I gene of E. coli that confers resistance to sulfonylureas (e.g., sulfometuron methyl) (for example, as described in Friden et al. Nucleic Acids Res. 13: 3979-3993 (1985); and LaRossa et al. J. Bacterial. 160: 391-394 (1984)).

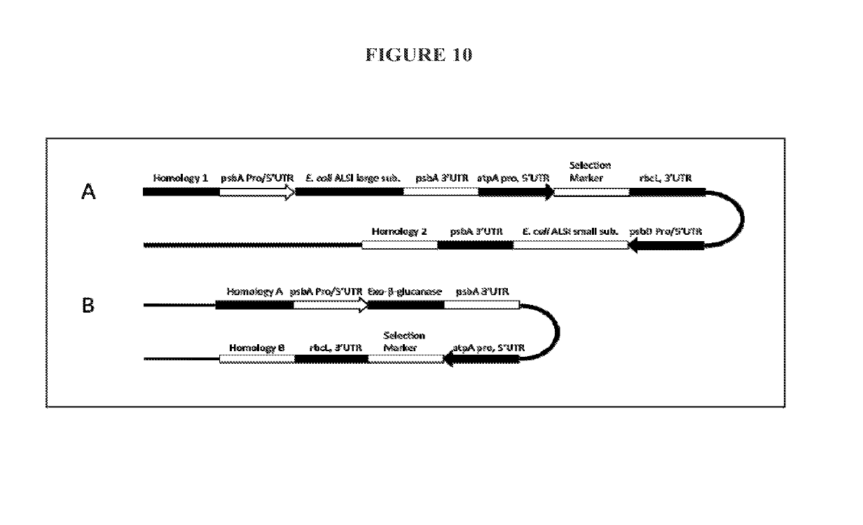

The E. coli ALS 1 large and small subunit open reading frames are codon biased to conform to the codon bias of the Chlamydomonas chloroplast genome using the oligo synthesis method detailed in Example 3. The two subunit genes are cloned in tandem in a chloroplast transformation vector (depicted in FIG. 10A) having the following organization: psbA locus homology region 1; psbA promoter and 5′ UTR; E. coli ALS I large subunit open reading frame; psbA 3′ UTR; psbD promoter and 5′UTR; E. coli ALS I small subunit open reading frame; psbA 3′ UTR; and psbA locus homology region 2. The chloroplast vector also includes a “selection marker”, the kanamycin resistance gene, which is regulated by the 5′ UTR and promoter sequence for the atpA gene from C. reinhardtii and the 3′ UTR sequence for the rbcL gene from C. reinhardtii. The transgene cassette is targeted to the psbA locus of C. reinhardtii via the homology regions 1 and 2. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations are carried out on C. reinhardtii strain 137c (mt+). Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty mls of cells are harvested by centrifugation at 4,000×g at 23° C. for 5 min. The supernatant is decanted and cells are resuspended in 4 ml TAP medium and spread on TAP plates that include (100 μg/ml) kanamycin or glyphosate for subsequent chloroplast transformation by particle bombardment (Cohen et al., Meth. Enzymol. 297: 192-208, 1998).

Following particle bombardment the number of transformants recovered from each type of selection is compared. Cells selected on kanamycin or glyphosate are replica plated on TAP plates that contain different concentrations of glyphosate to determine the level of glyphosate resistance in glyphosate and kanamycin selected cells.

PCR is used to identify transformed strains. For PCR analysis. 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algae lysate in EDTA is added to provide template for reaction. Magnesium concentration is varied to compensate for amount and concentration of algae lysate in EDTA added. Annealing temperature gradients are employed to determine optimal annealing temperature for specific primer pairs.

To identify strains that contain the ALS I genes, a primer pair is used in which one primer anneals to a site within the psbA 5′UTR or psbD 5′UTR and the other primer anneals within the ALS I large or small subunit coding region. Desired clones are those that yield a PCR product of expected size. To determine the degree to which the endogenous gene locus is displaced (heteroplasmic vs. homoplasmic), a PCR reaction containing two sets of primer pairs is employed. The first pair of primers amplifies the endogenous chloroplast genome locus targeted by the expression vector. The second pair of primers amplifies a constant, or control, region of the chloroplast genome that is not targeted by the expression vector, and should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is 30 or more to increase sensitivity. The most desired clones are those that yield a product for the constant region but not for the endogenous gene locus. Desired clones are also those that give weak-intensity endogenous locus products relative to the control reaction.

### Example 8

This example provides a herbicide resistant alga that can be grown in the presence of a herbicide for the production and isolation of a biomolecule.

A glyphosate resistant Chlamydomonas reinhardtii transformant of Example 3, exhibiting resistance to at least 1 mM glyphosate, or at least 10 mM glyphosate, is further transformed with a gene encoding a protein for biomass degradation.

In this example a nucleic acid encoding exo-β-glucanase from T. viride (SEQ ID NO: 60) (corresponding amino acid sequence as SEQ ID NO: 59) is introduced into the glyphosate resistant C. reinhardtii having the codon biased CP4 gene integrated into the chloroplast genome at the psbA locus (Example 3). Transforming DNA is depicted in FIG. 10B. The segment labeled “psbA Pro/5′ UTR” is the 5′ UTR and promoter sequence for the psbA gene from C. reinhardtii, the segment labeled “psbA 3′ UTR” contains the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the psbA loci of C. reinhardtii via the segments labeled “Homology A” and “Homology B,” which are identical to sequences of DNA flanking the psbA locus on the 5′ and 3′ sides, respectively. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

Chloroplast transformation is carried out on glyphosate-resistant C. reinhardtii strains from Example 3 by growing the cells to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty mls of cells are harvested by centrifugation at 4,000×g at 23° C. for 5 min. The supernatant is decanted and cells are resuspended in 4 ml TAP medium for subsequent chloroplast transformation by particle bombardment (Cohen et al., Meth. Enzymol. 297: 192-208, 1998). All transformations are carried out under kanamycin selection (150 μg/ml).

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algae lysate in EDTA is added to provide template for reaction. Magnesium concentration is varied to compensate for amount and concentration of algae lysate in EDTA added. Annealing temperature gradients are employed to determine optimal annealing temperature for specific primer pairs.

To identify strains that contain the exo-β-glucanase gene, a primer pair is used in which one primer anneals to a site within the psbA 5′UTR and the other primer anneals within the exo-β-glucanase coding segment. Desired clones are those that yield a PCR product of expected size. To determine the degree to which the endogenous gene locus is displaced (heteroplasmic vs. homoplasmic), a PCR reaction containing two sets of primer pairs is employed. The first pair of primers amplifies the endogenous locus targeted by the expression vector and consists of a primer that anneals within the psbA 5′UTR and one that anneals within the psbA coding region. The second pair of primers amplifies a constant, or control, region of the chloroplast genome that is not targeted by the expression vector, and should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube: however, the pair for the endogenous locus is 5× the concentration of the constant pair. The number of cycles used is 30 or more to increase sensitivity. The most desired clones are those that yield a product for the constant region but not for the endogenous gene locus. Desired clones are also those that give weak-intensity endogenous locus products relative to the control reaction.

To ensure that the presence of the exo-β-glucanase-encoding gene will lead to expression of the exo-β-glucanase protein in herbicide-grown cells, a transformant is selected that is homoplastic for the exo-β-glucanase-encoding gene and resistant to at least 1 mM glyphosate. TAP medium containing the highest concentration of glyphosate that will allow for unimpaired growth of the C. reinhardtii host cells is used for the growth of the doubly transformed C. reinhardtii cells.

Briefly, a 500 ml algal cell culture that includes glyphosate is grown to mid to late log phase (approximately 5×10⁶ cells per ml) and harvested by centrifugation at 4000×g at 4° C. for 15 min. The supernatant is decanted and the cells are resuspended in 10 ml of lysis buffer (100 mM Tris-HCl, pH=8.0, 300 mM NaCl, 2% Tween-20). Cells are lysed by sonication (10×30 sec at 35% power), and the lysate is clarified by centrifugation at 14,000×g at 4° C. for 1 hour. The supernatant is removed and incubated with anti-FLAG antibody-conjugated agarose resin at 4° C. for 10 hours. Resin is separated from the lysate by gravity filtration and washed 3× with wash buffer (100 mM Tris-HCl, pH=8.0, 300 mM NaCl, 2% Tween-20). Exo-β-glucanase is eluted by incubation of the resin with elution buffer (TBS, 250 ug/ml FLAG peptide). The presence of exo-β-glucanase is determined by Western blot.

To determine whether the isolated enzyme is functional, A 20 μl aliquot of diluted enzyme is added into wells containing 40 μl of 50 mM NaAc buffer and a filter paper disk. After 60 minutes incubation at 50° C., 120 μl of DNS is added to each reaction and incubated at 95° C. for 5 minutes. Finally, a 36 μl aliquot of each sample is transferred to the wells of a flat-bottom plate containing 160 μl water. The absorbance at 540 nm is measured. The results for the glyphosate resistant transformed strain determine whether the enzyme isolated from a herbicide-containing culture is functional.

### Example 9

This example provides the prokaryotic alga Synechocystis sp. Strain PCC6803 transformed with a gene conferring glyphosate resistance.

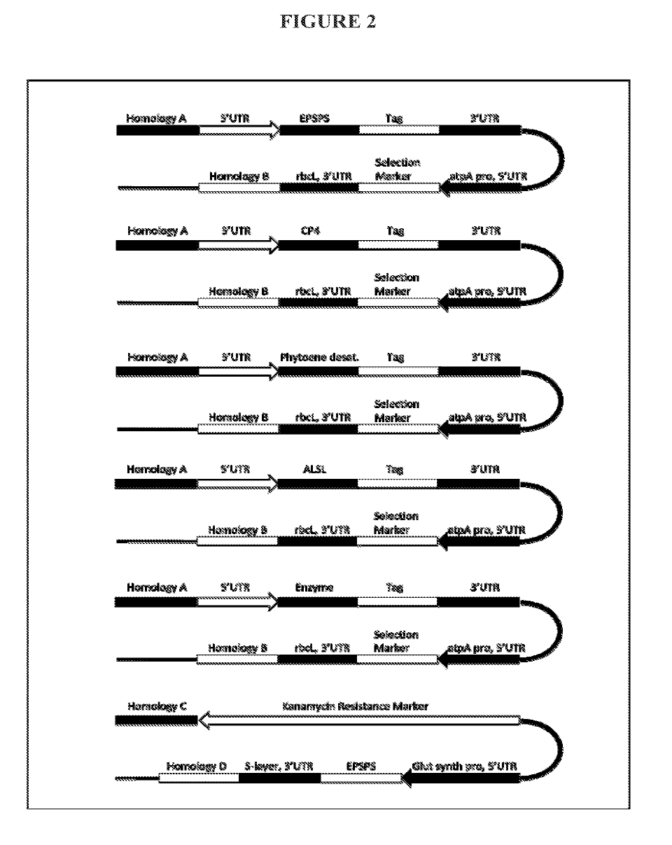

As depicted in FIG. 2F, a construct that includes an EPSPS encoding nucleotide sequence from Escherichia coli (SEQ ID NO: 66) is operably linked to the Synechocystis sp. Strain PCC6803 glutamine synthetase promoter and the 3′UTR/terminator sequence from the S-layer gene in Lactobacillus brevis. The E. coli EPSPS gene is modified by site-directed mutagenesis such that the glycine residue at position 96 is changed to alanine and the alanine residue at position 183 is changed to threonine (SEQ ID NO: 67) to confer glyphosate resistance. The construct also includes a bacterial selectable marker, the kanamycin resistance gene. The EPSPS gene and regulatory sequences are targeted to the psbY locus of Synechocystis via the segments labeled “Homology C” and “Homology D,” which are identical to sequences of DNA flanking the psbY locus on the 5′ and 3′ sides, respectively. All DNA manipulations are carried out essentially as described by Sambrook et al., Molecular Cloning: A Laboratory Manual (Cold Spring Harbor Laboratory Press 1989) and Cohen et al., Meth. Enzymol. 297, 192-208, 1998.

For transformation with the herbicide resistance gene, Synechocystis sp. strain 6803 is grown to a density of approximately 2×10⁸ cells per ml and harvested by centrifugation. The cell pellet is re-suspended in fresh BG-11 medium (ATCC Medium 616) at a density of 1×10⁹ cells per ml and used immediately for transformation. One-hundred microliters of these cells are mixed with 5 ul of a mini-prep solution containing the construct and the cells are incubated with light at 30° C. for 4 hours. This mixture is then plated onto nylon filters resting on BG-11 agar and grown for 12-18 hours. The filters are then transferred to BG-11 agar+TES+10 μg/ml kanamycin and allowed to grow until colonies appear, typically within 7-10 days.

Colonies are then picked into BG-11 liquid media containing 10 μg/ml kanamycin and grown for 5 days. Cells are then harvested for PCR analysis to determine the presence of the exogenous insert. Western blots may be performed (essentially as described in Example 10) to determine expression levels of the protein(s) encoded by the inserted construct.

### Example 10

This example demonstrates transformation of an algal chloroplast with a gene encoding homologous EPSP synthase, mutated to a form that confers resistance to glyphosate, to provide a glyphosate resistant alga.

The amino acid sequence of 5-enolpyruvylshikimate-3-phosphate synthase (EPSPS) of Chlamydomonas reinhardtii (Genbank Accession number XP_{—}001702942, GI: 159489926 (SEQ ID NO: 1)) was modified to obtain the mature C. reinhardtii EPSPS (SEQ ID NO: 58) by using homology with plant EPSPS protein sequences and the predicted cleavage site for chloroplast transit peptides identified using a program for predicting transit peptides and their cleavage sites (ChloroP, available at the URL link cbs.dur.dk/services/ChloroP/)) and was codon-optimized (SEQ ID NO: 16), in which the codon usage reflects the chloroplast genome codon bias of Chlamydomonas reinhardtii (Franklin et al. Plant J. 30: 733-744 (2002); Mayfield et al. Proc. Natl Acad Sci. USA 100: 438-442 (2003); see U.S. Patent Application Publication No. 2004/0014174). The codon-optimized sequence was used to synthesize a codon-optimized mature C. reinhardtii EPSPS coding sequence according to the oligo assembly method of Stemmer et al. (Gene 164: 49-53 (1995)). It is understood that PCR conditions can be modified with regard to reagent concentrations, temperatures, duration of each step, cycle number, etc., to optimize production of the desired polynucleotide.

Briefly, approximately 65 oligonucleotides were synthesized to span the approximately 1,335 bp nucleotide sequence encoding the mature codon optimized and doubly mutated C. reinhardtii EPSPS gene. The oligos were designed to incorporate optimized C. reinhardtii chloroplast codons and mutated amino acid codons. The oligos are 40 nucleotides in length, and comprise sequences from both strands of the gene, such that the oligos from opposite strands overlap one another and hybridize to one another in the regions of overlap. In the gene assembly PCR reactions, regions where there was no overlap (regions that are single-stranded when the full set of oligos is hybridized) were filled-in by polymerase. The outermost (5′most) oligos from each strand incorporate unique restriction sites for further cloning. The gene assembly PCR step was performed for 30-65 cycles, with the conditions optimized for production of a 1.335 kb full-length gene product. In one instance, PCR reactions for gene assembly were performed using 0.2 micromolar each oligo in a reaction mix containing 10 mM Tris-HCl, pH 9.0, 0.1% Triton X-100, 2.2 mM MgCl2, 50 mM KCl, 0.2 mM each of dATP, dCTP, dGTP, and dTTP, and 1 unit of Taq polymerase. Thirty cycles were performed of 30 seconds at 94 degrees C., 30 seconds at 52 degrees C., and 30 seconds at 72 degrees C.

The gene assembly PCR product was confirmed by gel electrophoresis of an aliquot of the PCR reaction, and then the gene assembly PCR reaction was diluted 40-fold into a 100 microliter PCR reaction that included the two outermost primers (the 5′ most primers of either strand) at I micromolar each, 10 mM Tris-HCl, pH 9.0, 0.1% Triton X-100, 2.2 mM MgCl2, 50 mM KCl, 0.2 mM each of dATP, dCTP, dGTP, and dTTP, and 1 unit of Taq polymerase. For gene amplification, 20 cycles were performed of 30 seconds at 94 degrees C., 30 seconds at 50 degrees C., 70 seconds at 72 degrees C. Following the amplification reactions, the PCR product was purified by phenol and chloroform extraction, ethanol precipitated, and digested with the enzymes recognizing the unique restriction sites at either end of the gene amplification product.

The digest was electrophoresed and the digested gene product was gel-purified prior to cloning the codon-optimized EPSPS gene into chloroplast cloning vector as depicted in FIG. 2A that includes the segment labeled “5′ UTR” that can be the promoter sequence for the psbA, psbD, or atpA gene from C. reinhardtii and the segment labeled “3′ UTR” for the psbA gene from C. reinhardtii. A Metal Affinity Tag (MAT), Tobacco etch virus (TEV) protease cleavage site and Flag antibody epitope is encoded at the 3′ end of the EPSPS gene and is labeled as “Tag”. The transgene cassette was targeted to the 3HB locus of C. reinhardtii via the segments labeled “Homology A” and “Homology B,” which are identical to sequences of DNA flanking the 3HB locus on the 5′ and 3′ sides, respectively. A kanamycin resistance gene from bacteria was used as the “Selection Marker”, which is regulated by the 5′ UTR and promoter sequence for the atpA gene from C. reinhardtii and the 3′ UTR sequence for the rbcL gene from C. reinhardtii. The codon-optimized mature C. reinhardtii EPSPS coding sequence was modified by site-directed mutagenesis such that the glycine residue at position 163 of the precursor protein (the form that includes the transit peptide) was changed to alanine (SEQ ID NO: 19 encoded by SEQ ID NO: 18), or modified such that the alanine residue at position 252 was changed to threonine (SEQ ID NO:21 encoded by SEQ ID NO:20) or was modified at both positions 163 and 252 (SEQ ID NO:23 encoded by SEQ ID:22). These amino acid positions correspond to positions 101 and 192 of the amino acid sequence of the predicted mature EPSPS protein (based on analogy of the C. reinhardtii EPSPS sequence to that of other mature EPSP sequences (see SEQ ID NO. 1 of U.S. Pat. No. 6,225,114). The mutations were introduced by PCR reactions using primers that incorporate the codon mutations, or by synthesis of a gene using the oligo assembly method of Stemmer et al. (Gene 164: 49-53 (1995)) outlined in the above examples, in which the oligos incorporated the mutated codon sequences. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297: 192-208, 1998.

All transformations were carried out on C. reinhardtii strain cc1690 (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells were resuspended in 4 ml TAP medium and spread on TAP plates that included (100 μg/ml) kanamycin, for subsequent chloroplast transformation by particle bombardment (Cohen et al., supra, 1998).

PCR was used to identify transformed strains (see U.S. Patent Application Publication No. 2009/0253169). For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water was prepared. Algal lysates in EDTA were added to provide template for the reactions. Magnesium concentration was varied to compensate for amount and concentration of algae lysate in EDTA added. Annealing temperature gradients were employed to determine optimal annealing temperature for specific primer pairs.

To identify strains that contain the EPSPS gene, a primer pair was used in which one primer anneals to a site within the psbD 5′UTR and the other primer anneals within the EPSPS coding segment. Desired clones were those that yield a PCR product of the expected size for the psbD 5′UTR linked to the recombinant EPSPS gene. To determine the degree to which the endogenous gene locus was displaced (heteroplasmic vs. homoplasmic), a PCR reaction consisting of two sets of primer pairs in the same reaction was employed. The first pair of primers amplifies the endogenous locus targeted by the expression vector and consists of a first primer that anneals within the psbD 5′UTR and a second primer that anneals within the psbD coding region. This primer pair only amplifies the psbD region of a chloroplast genome in which the EPSPS gene construct has not been integrated. The second pair of primers amplifies a constant, or control, region that is not targeted by the expression vector, and should produce a product of expected size whether or not the recombinant resistance gene is integrated into the chloroplast genome. This reaction was to confirm that the absence of a PCR product from the endogenous locus does not result from cellular and/or other contaminants that inhibited the PCR reaction. Concentrations of the primer pairs were varied so that both reactions work in the same tube; however, the pair for the endogenous locus is 5× the concentration of the constant pair. The number of cycles used was >30 to increase sensitivity. The most desired clones are those that yielded a product for the constant region but not for the endogenous gene locus. Desired clones were also those that gave weak-intensity endogenous locus products relative to the control reaction.

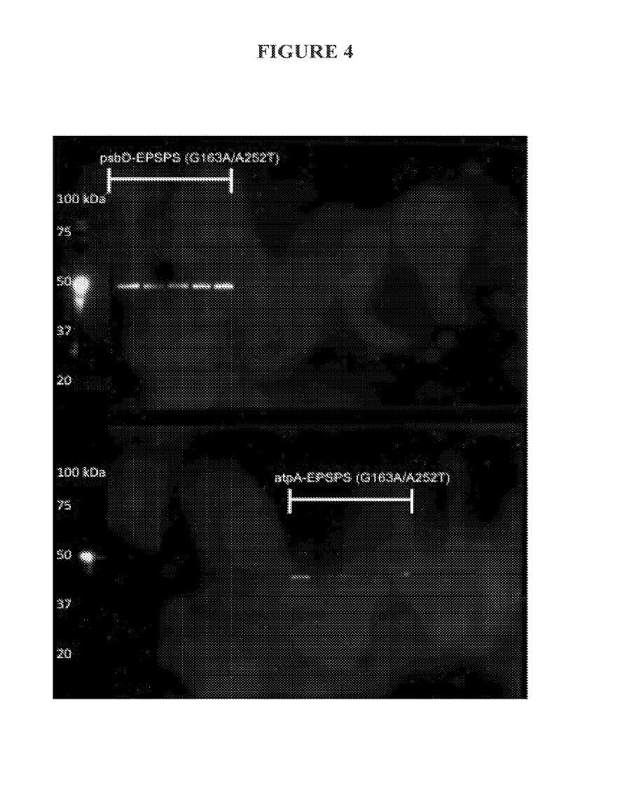

Patches of algae cells growing on TAP agar plates were lysed by resuspending cells in 50 μl of 1×SDS sample buffer with reducing agent (BioRad). Samples were then boiled and run on a 10% Bis-tris polyacrylamide gel (BioRad) and transferred to PVDF membranes using a Trans-blot semi-dry blotter (BioRad) according to the manufacturer's instructions. Membranes were blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes were washed four times with TBST, then developed with Supersignal West Dura chemiluminescent substrate (Thermo Scientific) and imaged using a CCD camera (Alpha Innotech). Expression resulted from the double mutated C. reinhardtii EPSPS driven by the psbD and atpA promoter regions is shown in FIG. 4.

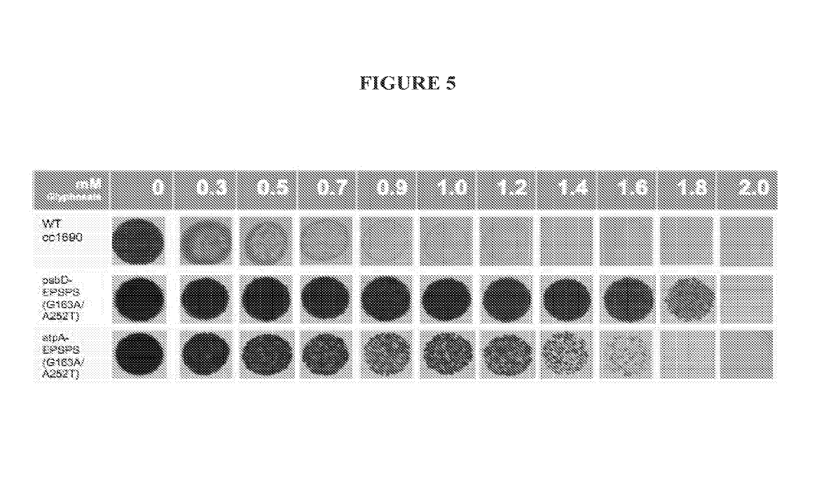

To characterize the effect of expressing the double mutated C. reinhardtii EPSPS directly in the chloroplast, engineered strains, along with wild type C. reinhardtii cc1690 (mt+), were plated on HSM plates with increasing amounts of glyphosate (0-2 mM). Wild type C. reinhardtii cc1690 was sensitive to approximately 1 mM glyphosate whereas the psbD-EPSPS (G163A/A252T) and atpA-EPSPS (G163A/A252T) engineered strains were sensitive at approximately 1.8 and 1.6 mM glyphosate, respectively. Results are shown in FIG. 5.

### Example 11

This example provides a eukaryotic alga genetically engineered to have two recombinant polynucleotides that confer resistance to two herbicides.

A Chlamydomonas nuclear transformant of Example 14 or 15, transformed with a homologous mutant EPSPS gene that confers resistance to glyphosate, is used as a host cell for chloroplast transformation with mutant forms of the large subunit of the acetolactate synthase, ALS, gene of C. reinhardtii that confers resistance to sulfonylureas (e.g., chlorsulfuron), imidazolinones (e.g., imazaquin), and pyrimidinylcarboxylate herbicides (e.g., pyriminabac) (Friden et al. Nucleic Acids Res. 13: 3979-3993 (1985); LaRossa et al. J. Bacteriol. 160: 391-394 (1984); Shimizu et al. Plant Physiol. 147:1976-1983 (2008)).

The amino acid sequence of acetolactate synthase large subunit of Chlamydomonas reinhardtii (Genbank Accession number AAC03784, GI: 2906139 (SEQ ID NO:61)) is modified to obtain the mature C. reinhardtii ALS large subunit (SEQ ID NO:62) by using homology with plant ALS protein sequences and the predicted cleavage site for chloroplast transit peptides identified using a program for predicting transit peptides and their cleavage sites (ChloroP, available at the URL link cbs.dur.dk/services/ChloroP/)) and is converted to DNA sequence (SEQ ID NO:63), in which the codon usage reflects the chloroplast genome codon bias of Chlamydomonas reinhardtii (Franklin et al. Plant J. 30: 733-744 (2002); Mayfield et al. Proc. Natl. Acad Sci. USA 100: 438-442 (2003); sec U.S. Patent Application Publication No. 2004/0014174). The codon-optimized sequence is used to synthesize a codon-optimized mature C. reinhardtii ALS large subunit coding sequence according to the oligo assembly method in Example 3. It is understood that PCR conditions can be modified with regard to reagent concentrations, temperatures, duration of each step, cycle number, etc., to optimize production of the desired polynucleotide.

The codon-optimized ALS large subunit gene is cloned into the chloroplast cloning vector depicted in FIG. 2D that includes the segment labeled “5′ UTR” that can be the promoter sequence for the psbA, psbD, or atpA gene from C. reinhardtii and the segment labeled “3′ UTR” for the psbA gene from C. reinhardtii. A Metal Affinity Tag (MAT), Tobacco etch virus (TEV) protease cleavage site and Flag antibody epitope is encoded at the 3′ end of the EPSPS gene and is labeled as “Tag”. The transgene cassette is targeted to the 3HB locus of C. reinhardtii via the segments labeled “Homology A” and “Homology B,” which are identical to sequences of DNA flanking the 3HB locus on the 5′ and 3′ sides, respectively. A kanamycin resistance gene from bacteria is used as the “Selection Marker”, which is regulated by the 5′ UTR and promoter sequence for the atpA gene from C. reinhardtii and the 3′ UTR sequence for the rbcL gene from C. reinhardtii. The codon-optimized mature C. reinhardtii ALS large subunit coding sequence is modified by site-directed mutagenesis such that the proline residue at position 198 of the precursor protein (the form that includes the transit peptide) is changed to serine, the tryptophan residue at position 580 is changed to leucine, and the serine residue at position 666 is changed to isoleucine (SEQ ID NO: 65 encoded by SEQ ID NO: 64). The single mutants are also generated. The mutations are introduced by PCR reactions using primers that incorporate the codon mutations, or by synthesis of a gene using the oligo assembly method of Stemmer et al. (Gene 164: 49-53 (1995)) outlined in the above examples, in which the oligos incorporate the mutated codon sequences. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297: 192-208, 1998.

Transformations are carried out on strains generated in Examples 14 and 15. Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells are resuspended in 4 ml TAP medium and spread on TAP plates that include (100 μg/ml) kanamycin, for subsequent chloroplast transformation by particle bombardment (Cohen et al., supra, 1998).

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl2, dNTPs, PCR primer pair(s), DNA polymerase, and water is prepared. Algae lysate in EDTA is added to provide template for reaction. Magnesium concentration is varied to compensate for amount and concentration of algae lysate in EDTA added. Annealing temperature gradients are employed to determine optimal annealing temperature for specific primer pairs.

To identify strains that contain the ALS large subunit gene, a primer pair is used in which one primer anneals to a site within the psbD 5′UTR and the other primer anneals within the ALS large subunit coding region. Desired clones are those that yield a PCR product of expected size. To determine the degree to which the endogenous gene locus is displaced (heteroplasmic vs. homoplasmic), a PCR reaction containing two sets of primer pairs is employed. The first pair of primers amplifies the endogenous chloroplast genome locus targeted by the expression vector. The second pair of primers amplifies a constant, or control, region of the chloroplast genome that is not targeted by the expression vector, and should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is 30 or more to increase sensitivity. The most desired clones are those that yield a product for the constant region but not for the endogenous gene locus. Desired clones are also those that give weak-intensity endogenous locus products relative to the control reaction.

### Example 12

This example provides an herbicide resistant alga that can be grown in the presence of an herbicide for the production and isolation of a biomolecule.

A glyphosate resistant Chlamydomonas reinhardtii transformant of Example 14 or 15 exhibiting resistance to at least 1 mM glyphosate, or at least 6 mM glyphosate, is further transformed with a gene encoding an industrial enzyme, therapeutic protein, or fuel molecule-producing enzyme.

A representative biomolecule is the biomass degrading enzyme cellobiohydrolase I from T. viride. The amino acid sequence of cellobiohydrolase I from T. viride (Genbank Accession number AAQ76092, GI: 34582632 (SEQ ID NO: 59)) is codon optimized to reflect the chloroplast genome codon bias of Chlamydomonas reinhardtii (Franklin et al. Plant J. 30: 733-744 (2002); Mayfield et al. Proc. Natl. Acad Sci. USA 100: 438-442 (2003); see U.S. Patent Application Publication No. 2004/0014174). The codon-optimized sequence (SEQ ID NO: 60) is used to synthesize a codon-optimized T. viride cellobiohydrolase according to the oligo assembly method of Stemmer et al. (Gene 164: 49-53 (1995)). In this example the nucleic acid encoding cellobiohydrolase from T. viride is introduced into a strain of C. reinhardtii having the EPSPS cDNA or genomic version of the gene integrated in the genome where the overexpressed wild type or mutant EPSPS protein confers glyphosate resistance (Example 9 or 10). It is understood that PCR conditions can be modified with regard to reagent concentrations, temperatures, duration of each step, cycle number, etc., to optimize production of the desired polynucleotide.

The cellobiohydrolase gene (SEQ ID NO: 60) is cloned into a vector depicted in FIG. 2E that includes the segment labeled “5′ UTR” that can be the promoter sequence for the psbA, psbD, or atpA gene from C. reinhardtii and the segment labeled “3′ UTR” for the psbA gene from C. reinhardtii. The segment labeled “Enzyme” represents the T. viride cellobiohydrolase gene or any industrial enzyme, therapeutic protein, or fuel molecule-producing enzyme. A Metal Affinity Tag (MAT), Tobacco etch virus (TEV) protease cleavage site and Flag antibody epitope is encoded at the 3′ end of the representative enzyme and is labeled as “Tag”. A kanamycin resistance gene from bacteria is used as the “Selection Marker”, which is regulated by the 5′ UTR and promoter sequence for the atpA gene from C. reinhardtii and the 3′ UTR sequence for the rbcL gene from C. reinhardtii. The transgene cassette is targeted to the 3HB locus of C. reinhardtii via the segments labeled “Homology A” and “Homology B,” which are identical to sequences of DNA flanking the 3HB locus on the 5′ and 3′ sides, respectively. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

Transformation is carried out on strains generated in Examples 14 and 15. Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells are resuspended in 4 ml TAP medium and spread on TAP plates that include (100 μg/ml) kanamycin, for subsequent chloroplast transformation by particle bombardment (Cohen et al., supra, 1998).

PCR is used to identify transformed strains (see U.S. Patent Application Publication No. 2009/0253169). For PCR analysis, 10 algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algal lysates in EDTA are added to provide template for the reactions. Magnesium concentration is varied to compensate for amount and concentration of algae lysate in EDTA added. Annealing temperature gradients are employed to determine optimal annealing temperature for specific primer pairs.

To identify strains that contain the cellobiohydrolase gene, a primer pair is used in which one primer anneals to a site within the psbD 5′UTR and the other primer anneals within the cellobiohydrolase coding segment. Desired clones are those that yield a PCR product of the expected size for the psbD 5′UTR linked to the recombinant cellobiohydrolase gene. To determine the degree to which the endogenous gene locus is displaced (heteroplasmic vs. homoplasmic), a PCR reaction consisting of two sets of primer pairs in the same reaction is employed. The first pair of primers amplifies the endogenous locus targeted by the expression vector and consists of a first primer that anneals within the psbD 5′UTR and a second primer that anneals within the psbD coding region. This primer pair only amplifies the psbD region of a chloroplast genome in which the cellobiohydrolase gene construct has not been integrated. The second pair of primers amplifies a constant, or control, region that is not targeted by the expression vector, and should produce a product of expected size whether or not the recombinant resistance gene is integrated into the chloroplast genome. This reaction is to confirm that the absence of a PCR product from the endogenous locus does not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is >30 to increase sensitivity. The most desired clones are those that yield a product for the constant region but not for the endogenous gene locus. Desired clones are also those that give weak-intensity endogenous locus products relative to the control reaction.

To ensure that the presence of cellobiohydrolase-encoding gene will lead to expression of the cellobiohydrolase protein in herbicide-grown cells, a transformant is selected that is homoplastic for the cellobiohydrolase-encoding gene and resistant to at least 1 mM glyphosate. HSM medium containing the highest concentration of glyphosate that will allow for unimpaired growth of the C. reinhardtii host cells is used for the growth of the doubly transformed C. reinhardtii cells.

Briefly, a 500 ml algal cell culture that includes glyphosate is grown to mid to late log phase (approximately 5×10⁶ cells per ml) and harvested by centrifugation at 4000×g at 4° C. for 15 min. The supernatant is decanted and the cells are resuspended in 10 ml of lysis buffer (100 mM Tris-HCl, pH=8.0, 300 mM NaCl, 2% Tween-20). Cells are lysed by sonication (10×30 sec at 35% power), and the lysate is clarified by centrifugation at 14,000×g at 4° C. for 1 hour. The supernatant is removed and incubated with anti-FLAG antibody-conjugated agarose resin at 4° C. for 10 hours. Resin is separated from the lysate by gravity filtration and washed 3× with wash buffer (100 mM Tris-HCl, pH=8.0, 300 mM NaCl, 2% Tween-20). Exo-β-glucanase is eluted by incubation of the resin with elution buffer (TBS, 250 ug/ml FLAG peptide). The presence of cellobiohydrolase is determined by Western blot.

To determine whether the isolated enzyme is functional, A 20 μl aliquot of diluted enzyme is added into wells containing 40 μl of 50 mM NaAc buffer and a filter paper disk. After 60 minutes incubation at 50° C., 120 μl of DNS is added to each reaction and incubated at 95° C. for 5 minutes. Finally, a 36 μl aliquot of each sample is transferred to the wells of a flat-bottom plate containing 1601 water. The absorbance at 540 nm is measured. The results for the glyphosate resistant transformed strain determine whether the enzyme isolated from an herbicide-containing culture is functional.

### Example 13

This example demonstrates transformation of an algal chloroplast with a gene encoding a heterologous phytoene desaturase to produce a norflurazon resistant alga.

The amino acid sequence of phytoene desaturase of a norflurazon resistant Synechococcus species strain 7942 (Genbank as Accession number CAA39004. GI: 48056 (SEQ ID NO: 4)) is converted to DNA sequence, in which the codon usage reflects the codon bias of the chloroplast genome of Chlamydomonas reinhardtii (Franklin et al. Plant J. 30: 733-744 (2002); Mayfield et al. Proc. Natl. Acad Sci. USA 100: 438-442 (2003); sec U.S. Patent Application Publication No. 2004/0014174). The codon-optimized sequence (SEQ ID NO: 57) is used to synthesize a codon-optimized C. reinhardtii phytoene desaturase coding sequence according to the oligo assembly method of Stemmer et al. (Gene 164: 49-53 (1995)).

The digested gene product is gel-purified prior to cloning the codon-optimized, E. coli EPSPS gene into chloroplast cloning vector depicted in FIG. 2C that includes the 5′ UTR and promoter sequence for the psbD gene from C. reinhardtii and the 3′ UTR for the psbA gene from C. reinhardtii. A Metal Affinity Tag (MAT), Tobacco etch virus (TEV) protease cleavage site and Flag antibody epitope is encoded at the 3′ end of the EPSPS cDNA and is labeled as “Tag”. The transgene cassette is targeted to the 3HB locus of C. reinhardtii via the segments labeled “Homology A” and “Homology B,” which are identical to sequences of DNA flanking the 3HB locus on the 5′ and 3′ sides, respectively. A kanamycin resistance gene from bacteria is used as the “Selection Marker”, which is regulated by the 5′ UTR and promoter sequence for the atpA gene from C. reinhardtii and the 3′ UTR sequence for the rbcL gene from C. reinhardtii. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

All transformations are carried out on C. reinhardtii strain cc1690 (mt+). Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells are resuspended in 4 ml TAP medium and spread on TAP plates that include (100 μg/ml) kanamycin, for subsequent chloroplast transformation by particle bombardment (Cohen et al., supra, 1998).

Following particle bombardment, some cells are selected on kanamycin selection (100 μg/ml) in which resistance is conferred by the kanamycin gene of the transformation vector (FIG. 2C). Other cells are selected on TAP plates that include to norflurazon. The number of transformants recovered from each type of selection is compared. Cells selected on kanamycin or glyphosate are replica plated on TAP plates that contain a range of concentrations of norflurazon to determine the level of norflurazon resistance in kanamycin selected cells.

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl2, dNTPs, PCR primer pair(s), DNA polymerase, and water is prepared. Algal lysates in EDTA are added to provide template for the reactions. Magnesium concentration is varied to compensate for amount and concentration of algae lysate in EDTA added. Annealing temperature gradients are employed to determine optimal annealing temperature for specific primer pairs.

To identify strains that contain the phytoene desaturase gene, a primer pair is used in which one primer anneals to a site within the psbD 5′UTR and the other primer anneals within the phytoene desaturase coding segment. Desired clones are those that yield a PCR product of the expected size for the psbD 5′UTR linked to the recombinant phytoene desaturase gene. To determine the degree to which the endogenous gene locus is displaced (heteroplasmic vs. homoplasmic), a PCR reaction consisting of two sets of primer pairs in the same reaction is employed. The first pair of primers amplifies the endogenous locus targeted by the expression vector and consists of a first primer that anneals within the psbD 5′UTR and a second primer that anneals within the psbA coding region. This primer pair only amplifies the psbA region of a chloroplast genome in which the phytoene desaturase gene construct has not been integrated. The second pair of primers amplifies a constant, or control, region that is not targeted by the expression vector, and should produce a product of expected size whether or not the recombinant resistance gene is integrated into the chloroplast genome. This reaction confirms that the absence of a PCR product from the endogenous locus does not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used is 30 or more to increase sensitivity. The most desired clones are those that yield a product for the constant region but not for the endogenous gene locus. Desired clones are also those that give weak-intensity endogenous locus products relative to the control reaction.

### Example 14

This example demonstrates transformation of an alga with a homologous cDNA gene encoding EPSP synthase that has been mutated to a form that confers resistance to glyphosate.

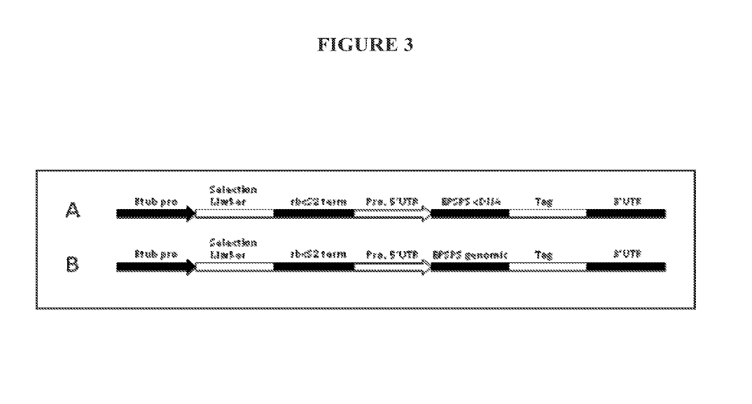

The nucleotide sequence of 5-enolpyruvylshikimate-3-phosphate synthase (EPSPS) of Chlamydomonas reinhardtii (Genbank as Accession number XM_{—}001702890, GI: 159489925 (SEQ ID NO: 24)) was modified by site-directed mutagenesis such that the glycine residue at position 163 of the precursor protein (the form that includes the transit peptide) was changed to alanine (SEQ ID NO: 27 encoded by SEQ ID NO: 26), or modified such that the alanine residue at position 252 was changed to threonine (SEQ ID NO: 29 encoded by SEQ ID NO: 28) or was modified at both positions 163 and 252 (SEQ ID NO: 31 encoded by SEQ ID: 30). These amino acid positions correspond to positions 101 and 192 of the amino acid sequence of the predicted mature EPSPS protein (based on analogy of the C. reinhardtii EPSPS sequence to that of other mature EPSP sequences (see SEQ ID NO. 1 of U.S. Pat. No. 6,225,114). The mutations were introduced by PCR reactions using primers that incorporate the codon mutations, or by synthesis of a gene using the oligo assembly method of Stemmer et al. (Gene 164: 49-53 (1995)) outlined in the above examples, in which the oligos incorporate the mutated codon sequences. The coding regions of the two single and double mutated C. reinhardtii EPSPS were cloned into the nuclear genome transformation vector depicted in FIG. 3A. The segment labeled “EPSPS cDNA” is the coding region of EPSPS, the segment labeled “Pro,5′ UTR” is the C. reinhardtii HSP70/rbcS2 promoter/5′ UTR with introns, and the segment labeled “3′ UTR” is the 3′UTR from C. reinhardtii rbcS2. The segment labeled “Selection Marker” is the hygromycin resistance gene with the β-tubulin promoter and rbcS2 terminator from C. reinhardtii. (Goldschmidt-Clermont and Rahire, J. Mol. Bio. 191: 421-432 (1986); Kozminski et al. Cell Motil. Cytoskel. 25: 158-170 (2005); Nelson et al. Mol. Cell. Biol. 14: 4011-4019 (1994): Marsh, Gene 32:481-485, (1984)). A Metal Affinity Tag (MAT), Tobacco etch virus (TEV) protease cleavage site and Flag antibody epitope is encoded at the 3′ end of the EPSPS cDNA and is labeled as “Tag”.

For these experiments, all transformations were carried out on C. reinhardtii cc1690 (mt+). Cells were grown and transformed via electroporation. Cells were grown to mid-log phase (approximately 2-6×10⁶ cells/ml). Tween-20 was added into cell cultures to a concentration of 0.05% before harvest to prevent cells from sticking to centrifugation tubes. Cells were spun down gently (between 2000 and 5000×g) for 5 min. The supernatant was removed and the cells resuspended in TAP+40 mM sucrose media. 1 to 2 μg of transforming DNA was mixed with ˜1×10⁸ cells on ice and transferred to electroporation cuvettes. Electroporation was performed with the capacitance set at 25 uF, the voltage at 800 V to deliver V/cm of 2000 and a time constant for 10-14 ms. Following electroporation, the cuvette was returned to room temperature for 5-20 min. Cells were transferred to 10 ml of TAP+40 mM sucrose and allowed to recover at room temperature for 12-16 hours with continuous shaking. Cells were then harvested by centrifugation at between 2000 g and 5000 g and resuspended in 0.5 ml TAP+40 mM sucrose medium. 0.25 ml of cells were plated on TAP+20 ug/ml hygromycin. All transformations were carried out under hygromycin selection (20 μg/ml) in which resistance was conferred by the gene encoded by the segment in FIG. 3A labeled “Selection Marker.” Transformed strains are maintained in the presence of hygromycin to prevent loss of the exogenous DNA.

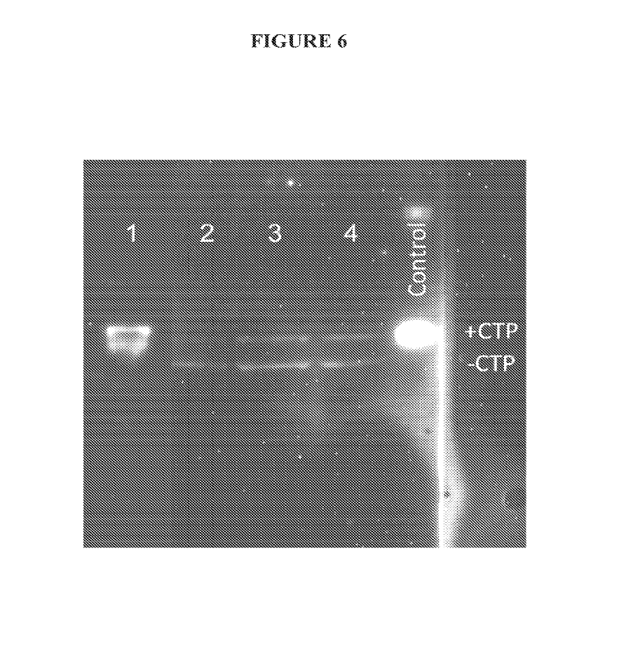

Patches of algae cells growing on TAP agar plates were lysed by resuspending cells in 50 μl of 1×SDS sample buffer with reducing agent (BioRad). Samples were then boiled and run on a 10% Bis-tris polyacrylamide gel (BioRad) and transferred to PVDF membranes using a Trans-blot semi-dry blotter (BioRad) according to the manufacturer's instructions. Membranes were blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes were washed four times with TBST, then developed with Supersignal West Dura chemiluminescent substrate (Thermo Scientific) and imaged using a CCD camera (Alpha Innotech). Expression resulted from the two single and double mutated C. reinhardtii EPSPS is shown in FIG. 6. Expression of the C. reinhardtii EPSPS WT cDNA in Escherichia coli is shown to indicate the presence and processing of the chloroplast targeting peptide (CTP).

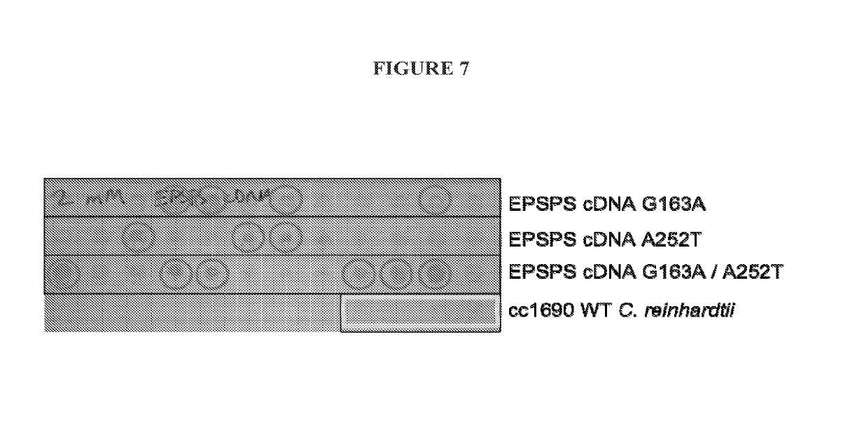

Random integration into the nuclear genome affects protein expression by a positional effect. To identify high expressing strains, hygromycin-resistant colonies were replica plated and grown in the presence of from 0 mM to 2 mM glyphosate to test transformants for glyphosate resistance. The percentage of highly resistant strains was indicative of the efficacy of the mutation(s) in conferring glyphosate resistance. Results are shown in FIG. 7. Engineering the double mutant G163A/A252T yielded more resistant strains. C. reinhardtii cc1690 WT was included as a negative control.

### Example 15

This example demonstrates transformation of an alga with a homologous genomic gene encoding EPSP synthase that has been mutated to a form that confers resistance to glyphosate.

The nucleotide sequence of 5-enolpyruvylshikimate-3-phosphate synthase (EPSPS) of Chlamydomonas reinhardtii (Genbank as Accession number DS496189. GI: 158270925 (SEQ ID NO:32) was amplified from genomic DNA and was modified by site-directed mutagenesis such that the glycine residue at position 163 of the precursor protein (the form that includes the transit peptide) was changed to alanine (SEQ ID NO: 35 encoded by SEQ ID NO: 34), or modified such that the alanine residue at position 252 was changed to threonine (SEQ ID NO: 37 encoded by SEQ ID NO: 36) or was modified at both positions 163 and 252 (SEQ ID NO: 39 encoded by SEQ ID: 38). These amino acid positions correspond to positions 101 and 192 of the amino acid sequence of the predicted mature EPSPS protein (based on analogy of the C. reinhardtii EPSPS sequence to that of other mature EPSP sequences (see Seq. ID No. I of U.S. Pat. No. 6,225,114). The mutations were introduced by PCR reactions using primers that incorporate the codon mutations, or by synthesis of a gene using the oligo assembly method of Stemmer et al. (Gene 164: 49-53 (1995)) outlined in the above examples, in which the oligos incorporate the mutated codon sequences. The wild type, the two single, and double mutated C. reinhardtii EPSPS genomic genes were cloned into the nuclear genome transformation vector depicted in FIG. 3B. The segment labeled “EPSPS genomic” is the genomic copy of the EPSPS gene including both introns and exons, the segment labeled “Pro, 5′ UTR” is the C. reinhardtii HSP70/rbcS2 promoter/5′ UTR with introns, and the segment labeled “3′ UTR” is the 3′UTR from C. reinhardtii rbcS2. The segment labeled “Selection Marker” is the hygromycin resistance gene with the β-tubulin promoter and rbcS2 terminator from C. reinhardtii. (Goldschmidt-Clermont and Rahire, J. Mol. Bio. 191: 421-432 (1986); Kozminski et al. Cell Motil. Cytoskel. 25: 158-170 (2005); Nelson et al. Mol. Cell. Biol. 14: 4011-4019 (1994); Marsh, Gene 32:481-485, (1984)). A Metal Affinity Tag (MAT), Tobacco etch virus (TEV) protease cleavage site and Flag antibody epitope is encoded at the 3′ end of the EPSPS genomic DNA and is labeled as “Tag”.

For these experiments, all transformations were carried out on C. reinhardtii cc1690 (mt+). Cells were grown and transformed via electroporation. Cells were grown to mid-log phase (approximately 2-6×10⁶ cells/ml). Tween-20 was added into cell cultures to a concentration of 0.05% before harvest to prevent cells from sticking to centrifugation tubes. Cells were spun down gently (between 2000 and 5000×g) for 5 min. The supernatant was removed and the cells resuspended in TAP+40 mM sucrose media. 1 to 2 μg of transforming DNA was mixed with ˜1×10⁸ cells on ice and transferred to electroporation cuvettes. Electroporation was performed with the capacitance set at 25 uF, the voltage at 800 V to deliver V/cm of 2000 and a time constant for 10-14 ms. Following electroporation, the cuvette was returned to room temperature for 5-20 min. Cells were transferred to 10 ml of TAP+40 mM sucrose and allowed to recover at room temperature for 12-16 hours with continuous shaking. Cells were then harvested by centrifugation at between 2000 g and 5000 g and resuspended in 0.5 ml TAP+40 mM sucrose medium. 0.25 ml of cells were plated on TAP+20 ug/ml hygromycin. All transformations were carried out under hygromycin selection (20 μg/ml) in which resistance was conferred by the gene encoded by the segment in FIG. 2B labeled “Selection Marker.” Transformed strains are maintained in the presence of hygromycin to prevent loss of the exogenous DNA.

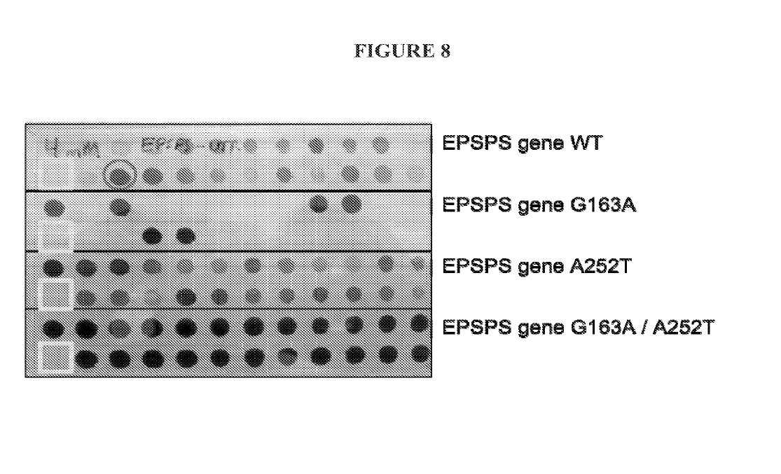

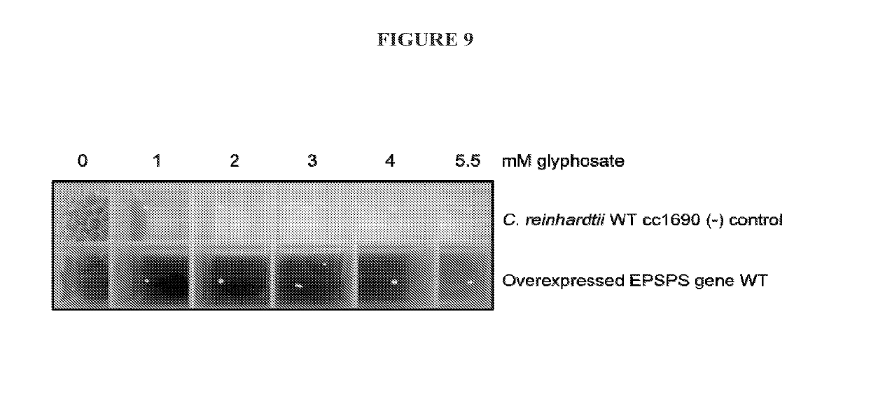

Random integration into the nuclear genome affects protein expression by a positional effect. To identify high expressing strains, hygromycin-resistant colonies were replica plated and grown in the presence of from 0 mM to 4 mM glyphosate to test transformants for glyphosate resistance. The percentage of highly resistant strains was indicative of the efficacy of the mutation(s) in conferring glyphosate resistance. Results are shown in FIG. 8. Engineering the double mutant G163A/A252T yielded more highly resistant strains. C. reinhardtii cc1690 WT was included as a negative control. Overexpression of a wild type copy of EPSPS was shown to also confer glyphosate resistance. To characterize resistance in liquid growth media, a liquid kill curve using glyphosate was performed on a strain in which a wild type copy of the C. reinhardtii EPSPS gene is overexpressed. C. reinhardtii cc1690 WT was included as a negative control. Results are shown in FIG. 9

### Example 16

This example provides an alga having a heterologous EPSP synthase that confers resistance to glyphosate, integrated into the chloroplast genome.

The amino acid sequence of the EPSPS gene of Escherichia coli (Genbank Accession number P0A6D3, GI: 67462163 (SEQ ID NO: 9)) was converted to a codon-optimized DNA sequence (SEQ ID NO: 8), in which the codon usage reflects the chloroplast codon bias of Chlamydomonas reinhardtii (Franklin et al. Plant J. 30: 733-744 (2002); Mayfield et al. Proc. Nail Acad Sci. USA 100: 438-442 (2003); see U.S. Patent Application Publication No. 2004/0014174). The codon-optimized E. coli EPSPS nucleotide sequence was used to synthesize a codon-optimized E. coli EPSPS gene according to the oligo assembly method of Stemmer et al. (Gene 164: 49-53 (1995)), as detailed above in Example 3 for the C. reinhardtii EPSPS gene.

The digested gene product was gel-purified prior to cloning the codon-optimized, E. coli EPSPS gene into chloroplast cloning vector depicted in FIG. 2A that includes the 5′ UTR and promoter sequence for the psbD gene from C. reinhardtii and the 3′ UTR for the psbA gene from C. reinhardtii. A Metal Affinity Tag (MAT), Tobacco etch virus (TEV) protease cleavage site and Flag antibody epitope was encoded at the 3′ end of the EPSPS gene and is labeled as “Tag”. The transgene cassette was targeted to the 3HB locus of C. reinhardtii via the segments labeled “Homology A” and “Homology B,” which are identical to sequences of DNA flanking the 3HB locus on the 5′ and 3′ sides, respectively. A kanamycin resistance gene from bacteria was used as the “Selection Marker”, which is regulated by the 5′ UTR and promoter sequence for the atpA gene from C. reinhardtii and the 3′ UTR sequence for the rbcL gene from C. reinhardtii. The codon-optimized mature E. coli EPSPS coding sequence was modified by site-directed mutagenesis such that the glycine residue at position 96 of the protein (the form that includes the transit peptide) was changed to alanine (SEQ ID NO: 11 encoded by SEQ ID NO: 10), or modified such that the alanine residue at position 183 was changed to threonine (SEQ ID NO: 13 encoded by SEQ ID NO: 12) or was modified at both positions 96 and 183 (SEQ ID NO: 15 encoded by SEQ ID: 14). The mutations were introduced by PCR reactions using primers that incorporate the codon mutations, or by synthesis of a gene using the oligo assembly method of Stemmer et al. (Gene 164: 49-53 (1995)) outlined in the above examples, in which the oligos incorporate the mutated codon sequences. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297: 192-208, 1998.

All transformations were carried out on C. reinhardtii strain cc1690 (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells were resuspended in 4 ml TAP medium and spread on TAP plates that include (100 μg/mil) kanamycin, for subsequent chloroplast transformation by particle bombardment (Cohen et al., supra, 1998).

PCR was used to identify transformed strains (see U.S. Patent Application Publication No. 2009/0253169). For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water was prepared. Algal lysates in EDTA were added to provide template for the reactions. Magnesium concentration was varied to compensate for amount and concentration of algae lysate in EDTA added. Annealing temperature gradients were employed to determine optimal annealing temperature for specific primer pairs.

To identify strains that contain the EPSPS gene, a primer pair was used in which one primer anneals to a site within the psbD 5′UTR and the other primer anneals within the EPSPS coding segment. Desired clones were those that yield a PCR product of the expected size for the psbD 5′UTR linked to the recombinant EPSPS gene. To determine the degree to which the endogenous gene locus was displaced (heteroplasmic vs. homoplasmic), a PCR reaction consisting of two sets of primer pairs in the same reaction was employed. The first pair of primers amplifies the endogenous locus targeted by the expression vector and consists of a first primer that anneals within the psbD 5′UTR and a second primer that anneals within the psbD coding region. This primer pair only amplifies the psbD region of a chloroplast genome in which the EPSP gene construct has not been integrated. The second pair of primers amplifies a constant, or control, region that is not targeted by the expression vector, and should produce a product of expected size whether or not the recombinant resistance gene is integrated into the chloroplast genome. This reaction was to confirm that the absence of a PCR product from the endogenous locus does not result from cellular and/or other contaminants that inhibited the PCR reaction. Concentrations of the primer pairs were varied so that both reactions work in the same tube; however, the pair for the endogenous locus is 5× the concentration of the constant pair. The number of cycles used was >30 to increase sensitivity. The most desired clones were those that yielded a product for the constant region but not for the endogenous gene locus. Desired clones were also those that give weak-intensity endogenous locus products relative to the control reaction.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-278. (canceled)

279. A method of growing an alga in a liquid culture, said method comprising:
obtaining a herbicide resistant alga comprising a recombinant nucleic acid molecule having a nucleic acid sequence encoding a polypeptide that confers herbicide resistance to an alga, wherein said nucleic acid sequence comprises:
(a) a nucleotide sequence of SEQ ID NO:78, SEQ ID NO:80, SEQ ID NO:82, SEQ ID NO:93, SEQ ID NO:94, or SEQ ID NO:95,
(b) a nucleotide sequence having more than 90% identity to said nucleotide sequence of (a) and further comprising nucleic acids that encode a G163A mutation, a A252T mutation, or both mutations relative to the corresponding positions of SEQ ID NO: 1, or
(c) a nucleotide sequence encoding a polypeptide sequence having more than 90% identity to an amino acid sequence selected from the group consisting of SEQ ID NOs: 79, 81, and 83, wherein said polypeptide sequence comprises a G163A mutation, a A252T mutation, or both mutations relative to the corresponding positions of SEQ ID NO: 1; and
growing said herbicide resistant alga in a liquid culture in the presence of said herbicide.

280. The method of claim 279, wherein said herbicide resistant alga is grown in a raceway pond, a photobioreactor, an open pond, a waterbed, a shallow pool, a reservoir, a tank, or a canal.

281. The method of claim 279, wherein said herbicide resistant alga is grown in the presence of glyphosate at a concentration between about 1 mM and about 6 mM.

282. The method of claim 279, wherein said alga is a cyanobacterium or a green alga of the order Chlorophyta.

283. The method of claim 279, wherein said alga is eukaryotic.

284. The method of claim 279, wherein said alga is selected from the group consisting of a Synechococcus sp., a Synechocystis sp., an Athrospira sp., an Anacytis sp. an Anabaena sp., a Nostoc sp., a Spirulina sp., a Fremyella sp., a Chlamydomonas sp., a Volvacales sp., a Dunaliella sp., a Scenedesmus sp., a Chlorella sp., and a Hematococcus sp.

285. The method of claim 284, wherein said alga is a Chlamydomonas sp. or a Synechocystis sp.

286. The method of claim 279, wherein said nucleic acid molecule is integrated into a nuclear genome of said alga.

287. The method of claim 285, wherein said nucleic acid molecule is codon-biased to reflect the codon bias of said nuclear genome.

288. The method of claim 279, wherein said nucleic acid molecule is integrated into a chloroplast genome of said alga.

289. The method of claim 285, wherein said nucleic acid molecule is codon-biased to reflect the codon bias of said chloroplast genome.

290. The method of claim 279, wherein said nucleotide sequence of (b) has more than 95% identity to said nucleotide sequence of (a).

291. The method of claim 290, wherein said nucleotide sequence of (b) has more than 99% identity to said nucleotide sequence of (a).

292. The method of claim 279, wherein said polypeptide has more than 95% identity to said amino acid sequence selected from the group consisting of SEQ ID NOs: 79, 81, and 83.

293. The method of claim 292, wherein said polypeptide has more than 99% identity to said amino acid sequence selected from the group consisting of SEQ ID NOs: 79, 81, and 83.

294. A method of making a herbicide resistant alga, comprising:
(a) transforming an alga with a polynucleotide comprising a nucleic acid sequence encoding a polypeptide that confers herbicide resistance to said alga, wherein said nucleic acid sequence comprises:
(i) a nucleotide sequence of SEQ ID NO:78, SEQ ID NO:80, SEQ ID NO:82, SEQ ID NO:93, SEQ ID NO:94, or SEQ ID NO:95;
(ii) a nucleotide sequence having more than 90% identity to said nucleotide sequence of (i) and further comprising nucleic acids that encode a G163A mutation, a A252T mutation, or both mutations relative to the corresponding positions of SEQ ID NO:1; or
(iii) a nucleotide sequence encoding a polypeptide sequence having more than 90% identity to an amino acid sequence selected from the group consisting of SEQ ID NOs: 79, 81, and 83, wherein said polypeptide sequence comprises a G163A mutation, a A252T mutation, or both mutations relative to the corresponding positions of SEQ ID NO: 1.

295. The method of claim 294, further comprising:
(b) growing said alga in the presence of herbicide, wherein said polypeptide confers herbicide resistance to said alga; and
(c) harvesting one or more biomolecules from said alga.

296. The method of claim 295, wherein said one or more biomolecules are selected from the group consisting of a biomass-degrading enzyme, a therapeutic protein, a nutritional molecule, a fuel product, and a combination thereof.

297. A herbicide resistant alga comprising a recombinant nucleic acid molecule comprising a nucleic acid sequence encoding a polypeptide that confers herbicide resistance to an alga, wherein said nucleic acid sequence comprises:
(a) a nucleotide sequence of SEQ ID NO:78, SEQ ID NO:80, SEQ ID NO:82, SEQ ID NO:93, SEQ ID NO:94, or SEQ ID NO:95;
(b) a nucleotide sequence having more than 90% identity to said nucleotide sequence of (a) and further comprising nucleic acids that encode a G163A mutation, a A252T mutation, or both mutations relative to the corresponding positions of SEQ ID NO: 1; or
(c) a nucleotide sequence encoding a polypeptide sequence having more than 90% identity to an amino acid sequence selected from the group consisting of SEQ ID NOs: 79, 81, and 83, wherein said polypeptide sequence comprises a G163A mutation, a A252T mutation, or both mutations relative to the corresponding positions of SEQ ID NO: 1.

298. The herbicide resistant alga of claim 297, wherein said alga is a cyanobacterium or a green alga of the order Chlorophyta.


SEQUENCE LISTING (100 sequences)

SEQ ID NO: 1 - protein, 512 aa, Chlamydomonas reinhardtii
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 2 - protein, 512 aa, Chlamydomonas reinhardtii
  VARIANT (163)..(163)
  VARIANT (252)..(252)
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 3 - protein, 455 aa, Agrobacterium sp.
MSHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 4 - protein, 474 aa, Synechococcus elongatus
MRVAIAGAGLAGLSCAKYLADAGHTPIVYERRDVLGGKVAAWKDEDGDWYETGLHIFFGAYPNMLQLFKELNIEDRLQWKSHSMIFNQPTKPGTYSRFDFPDIPAPINGVAAILSNNDMLTWEEKIKFGLGLLPAMIRGQSYVEEMDQYSWTEWLRKQNIPERVNDEVFIAMAKALNFIDPDEISATVVLTALNRFLQEKKGSMMAFLDGAPPERLCQPIVEHVQARGGDVLLNAPLKEFVLNDDSSVQAFRIAGIKGQEEQLIEADAYVSALPVDPLKLLLPDAWKAMPYFQQLDGLQGVPVINIHLWFDRKLTDIDHLLFSRSPLLSVYADMSNTCREYEDPDRSMLELVFAPAKDWIGRSDEDILAATMAEIEKLFPQHFSGENPARLRKYKIVKTPLSVYKATPGRQQYRPDQASPIANFFLTGDYTMQRYLASMEGAVLSGKLTAQAIIARQDELQRRSSGRPLAASQA

SEQ ID NO: 5 - DNA, 1332 bp, Unknown
  unknown bacterium
atggcgtgtttgcctgatgattcgggtccgcatgtcggccactccacgccacctcgccttgaccaggagccttgtaccttgagttcgcagaaaaccgtgaccgttacaccgcccaacttccccctcactggcaaggtcgcgccccccggctccaaatccattaccaaccgtgcgctgttgctggcggcattggccaagggcaccagccgtttgagcggtgcgctcaaaagcgatgacacgcgccacatgtcggtcgccctgcggcagatgggcgtcaccatcgacgagccggacgacaccacctttgtggtcaccagccaaggctcgctgcaattgccggcccagccgttgttcctcggcaacgctggcaccgccatgcgctttctcacggctgccgtggccaccgtgcaaggcaccgtggtactggacggcgacgagtacatgcaaaaacgcccgattggcccgctgctggctaccctgggccagaacggcatccaggtcgacagccccaccggttgcccaccggtcaccgtgcacggcatgggcaaggtccaggccaagcgtttcgagattgatggtggtttgtccagccagtacgtatcggccctgctgatgctcgcggcgtgcggcgaagcgccgattgaagtggcgctgaccggcaaggatatcggtgcccgtggctacgtggacctgaccctcgactgcatgcgtgccttcggggcccaggtggacgccgtggacgacaccacctggcgcgtcgcccccaccggctataccgcccatgattacctgatcgaacccgatgcgtccgccgccacgtatttgtgggccgcagaagtgctgaccggtgggcgtatcgacatcggcgtagccgcgcaggacttcacccagcccgacgccaaggcccaggccgtgattgcgcagttcccgaacatgcaagccacggtggtaggctcgcaaatgcaggatgcgatcccgaccctggcggtgctcgccgcgttcaacaacaccccggtgcgtttcactgaactggcgaacctgcgcgtcaaggaatgtgaccgcgtgcaggcgctgcacgatggcctcaacgaaattcgcccgggcctggcgaccatcgagggcgatgacctgctggtcgccagcgacccggccctggcaggcaccgcctgcaccgcactgatcgacacccacgccgaccatcgcatcgccatgtgctttgccctggccgggcttaaagtctcgggcattcgcattcaagacccggactgcgtggccaagacctaccctgactactggaaagcctggcccagcctgggcgttcacctaaacgac

SEQ ID NO: 6 - protein, 444 aa, Unknown
  Unknown bacterium
MACLPDDSGPHVGHSTPPRLDQEPCTLSSQKTVTVTPPNFPLTGKVAPPGSKSITNRALLLAALAKGTSRLSGALKSDDTRHMSVALRQMGVTIDEPDDTTFVVTSQGSLQLPAQPLFLGNAGTAMRFLTAAVATVQGTVVLDGDEYMQKRPIGPLLATLGQNGIQVDSPTGCPPVTVHGMGKVQAKRFEIDGGLSSQYVSALLMLAACGEAPIEVALTGKDIGARGYVDLTLDCMRAFGAQVDAVDDTTWRVAPTGYTAHDYLIEPDASAATYLWAAEVLTGGRIDIGVAAQDFTQPDAKAQAVIAQFPNMQATVVGSQMQDAIPTLAVLAAFNNTPVRFTELANLRVKECDRVQALHDGLNEIRPGLATIEGDDLLVASDPALAGTACTALIDTHADHRIAMCFALAGLKVSGIRIQDPDCVAKTYPDYWKAWPSLGVHLND

SEQ ID NO: 7 - protein, 444 aa, Petunia x hybrida
KPSEIVLQPIKEISGTVKLPGSKSLSNRILLLAALSEGTTVVDNLLSSDDIHYMLGALKTLGLHVEEDSANQRAVVEGCGGLFPVGKESKEEIQLFLGNAGTAMRPLTAAVTVAGGNSRYVLDGVPRMRERPISDLVDGLKQLGAEVDCFLGTKCPPVRIVSKGGLPGGKVKLSGSISSQYLTALLMAAPLALGDVEIEIIDKLISVPYVEMTLKLMERFGISVEHSSSWDRFFVRGGQKYKSPGKAFVEGDASSASYFLAGAAVTGGTITVEGCGTNSLQGDVKFAEVLEKMGAEVTWTENSVTVKGPPRSSSGRKHLRAIDVNMNKMPDVAMTLAVVALYADGPTAIRDVASWRVKETERMIAICTELRKLGATVEEGPDYCIITPPEKLNVTDIDTYDDHRMAMAFSLAACADVPVTINDPGCTRKTFPNYFDVLQQYSKH

SEQ ID NO: 8 - DNA, 1380 bp, Artificial Sequence
  Codon optimized sequence
atggtaccaatggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctggtactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttagcacctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgctggtaccggtgattacaaagacgacgacgataaatcaggtgaaaatctttactttcaaggtcataaccatagacacaaacataccggttaa

SEQ ID NO: 9 - protein, 459 aa, Escherichia coli
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAAGTGDYKDDDDKSGENLYFQGHNHRHKHTG

SEQ ID NO: 10 - DNA, 1380 bp, Artificial Sequence
  Codon optimized sequence
atggtaccaatggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctgcaactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttagcacctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgctggtaccggtgattacaaagacgacgacgataaatcaggtgaaaatctttactttcaaggtcataaccatagacacaaacataccggttaa

SEQ ID NO: 11 - protein, 459 aa, Escherichia coli
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAATAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAAGTGDYKDDDDKSGENLYFQGHNHRHKHTG

SEQ ID NO: 12 - DNA, 1380 bp, Artificial Sequence
  Codon optimized sequence
atggtaccaatggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctggtactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttaacgcctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgctggtaccggtgattacaaagacgacgacgataaatcaggtgaaaatctttactttcaaggtcataaccatagacacaaacataccggttaa

SEQ ID NO: 13 - protein, 459 aa, Escherichia coli
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLTPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAAGTGDYKDDDDKSGENLYFQGHNHRHKHTG

SEQ ID NO: 14 - DNA, 1380 bp, Artificial Sequence
  Codon optimized sequence
atggtaccaatggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctgcaactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttaacgcctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgctggtaccggtgattacaaagacgacgacgataaatcaggtgaaaatctttactttcaaggtcataaccatagacacaaacataccggttaa

SEQ ID NO: 15 - protein, 459 aa, Escherichia coli
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAATAMRPLAAALCLGSNDIVLTGEPRMKERPIGHLVDALRLGGAKITYLEQENYPPLRLQGGFTGGNVDVDGSVSSQFLTALLMTAPLTPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAAGTGDYKDDDDKSGENLYFQGHNHRHKHTG

SEQ ID NO: 16 - DNA, 1431 bp, Artificial Sequence
  Codon optimized sequence
atggtaccagtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctggaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttgctgttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacacggtaccggtgattataaagacgacgatgataaaagtggagaaaacttatactttcaaggtcataaccaccgtcacaaacataccggttaa

SEQ ID NO: 17 - protein, 476 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHGTGDYKDDDDKSGENLYFQGHNHRHKHTG

SEQ ID NO: 18 - DNA, 1431 bp, Artificial Sequence
  Codon optimized sequence
atggtaccagtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctgcaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttgctgttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacacggtaccggtgattataaagacgacgatgataaaagtggagaaaacttatactttcaaggtcataaccaccgtcacaaacataccggttaa

SEQ ID NO: 19 - protein, 476 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHGTGDYKDDDDKSGENLYFQGHNHRHKHTG

SEQ ID NO: 20 - DNA, 1431 bp, Artificial Sequence
  Codon optimized sequence
atggtaccagtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctggaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttacggttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacacggtaccggtgattataaagacgacgatgataaaagtggagaaaacttatactttcaaggtcataaccaccgtcacaaacataccggttaa

SEQ ID NO: 21 - protein, 476 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHGTGDYKDDDDKSGENLYFQGHNHRHKHTG

SEQ ID NO: 22 - DNA, 1431 bp, Artificial Sequence
  Codon optimized sequence
atggtaccagtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctgcaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttacggttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacacggtaccggtgattataaagacgacgatgataaaagtggagaaaacttatactttcaaggtcataaccaccgtcacaaacataccggttaa

SEQ ID NO: 23 - protein, 476 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHGTGDYKDDDDKSGENLYFQGHNHRHKHTG

SEQ ID NO: 24 - DNA, 1632 bp, Chlamydomonas reinhardtii
  misc_feature (1)..(9): Additional 5′ nucleotids
atgctcgagatgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcacaccggtgattataaggacgacgatgacaagagcggcgagaacctgtattttcagggccataaccaccgtcataagcacaccggttag

SEQ ID NO: 25 - protein, 543 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHTGDYKDDDDKSGENLYFQGHNHRHKHTG

SEQ ID NO: 26 - DNA, 1632 bp, Chlamydomonas reinhardtii
  misc_feature (1)..(9): Additional 5′ nucleotides
atgctcgagatgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcacaccggtgattataaggacgacgatgacaagagcggcgagaacctgtattttcagggccataaccaccgtcataagcacaccggttag

SEQ ID NO: 27 - protein, 543 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHTGDYKDDDDKSGENLYFQGHNHRHKHTG

SEQ ID NO: 28 - DNA, 1632 bp, Chlamydomonas reinhardtii
  misc_feature (1)..(9): Additional 5′ nucleotides
atgctcgagatgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcacaccggtgattataaggacgacgatgacaagagcggcgagaacctgtattttcagggccataaccaccgtcataagcacaccggttag

SEQ ID NO: 29 - protein, 543 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHTGDYKDDDDKSGENLYFQGHNHRHKHTG

SEQ ID NO: 30 - DNA, 1632 bp, Chlamydomonas reinhardtii
  misc_feature (1)..(9): Additional 5′ nucleotides
atgctcgagatgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcacaccggtgattataaggacgacgatgacaagagcggcgagaacctgtattttcagggccataaccaccgtcataagcacaccggttag

SEQ ID NO: 31 - protein, 543 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHTGDYKDDDDKSGENLYFQGHNHRHKHTG

SEQ ID NO: 32 - DNA, 4203 bp, Chlamydomonas reinhardtii
  misc_feature (4120)..(4200): Affinity tag
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactacgactacaaggacgacgacgacaagtccggcgagaacctgtactttcaggggcacaaccaccgccataagcacgtatagtga

SEQ ID NO: 33 - protein, 538 aa, Chlamydomonas reinhardtii
  MISC_FEATURE (513)..(538): Affinity tag
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHYDYKDDDDKSGENLYFQGHNHRHKHV

SEQ ID NO: 34 - DNA, 4203 bp, Chlamydomonas reinhardtii
  mutation (899)..(901)
  misc_feature (4120)..(4200): Affinity tag
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactacgactacaaggacgacgacgacaagtccggcgagaacctgtactttcaggggcacaaccaccgccataagcacgtatagtga

SEQ ID NO: 35 - protein, 538 aa, Chlamydomonas reinhardtii
  VARIANT (163)..(163)
  MISC_FEATURE (513)..(538): Affinity tag
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHYDYKDDDDKSGENLYFQGHNHRHKHV

SEQ ID NO: 36 - DNA, 4203 bp, Chlamydomonas reinhardtii
  mutation (2203)..(2205)
  misc_feature (4120)..(4200): Affinity tag
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactacgactacaaggacgacgacgacaagtccggcgagaacctgtactttcaggggcacaaccaccgccataagcacgtatagtga

SEQ ID NO: 37 - protein, 538 aa, Chlamydomonas reinhardtii
  VARIANT (252)..(252)
  MISC_FEATURE (513)..(538): Affinity tag
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHYDYKDDDDKSGENLYFQGHNHRHKHV

SEQ ID NO: 38 - DNA, 4203 bp, Chlamydomonas reinhardtii
  mutation (899)..(901)
  mutation (2203)..(2205)
  misc_feature (4120)..(4200): Affinity tag
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactacgactacaaggacgacgacgacaagtccggcgagaacctgtactttcaggggcacaaccaccgccataagcacgtatagtga

SEQ ID NO: 39 - protein, 538 aa, Chlamydomonas reinhardtii
  VARIANT (163)..(163)
  VARIANT (252)..(252)
  MISC_FEATURE (513)..(538): Affinity tag
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQHYDYKDDDDKSGENLYFQGHNHRHKHV

SEQ ID NO: 40 - protein, 430 aa, Chlamydomonas reinhardtii
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 41 - protein, 430 aa, Chlamydomonas reinhardtii
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAATAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 42 - protein, 430 aa, Chlamydomonas reinhardtii
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLTPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 43 - protein, 430 aa, Chlamydomonas reinhardtii
  MISC_FEATURE (1)..(3): Additional amino acids
MVPMESLTLQPIARVDGTINLPGSKSVSNRALLLAALAHGKTVLTNLLDSDDVRHMLNALTALGVSYTLSADRTRCEIIGNGGPLHAEGALELFLGNAATAMRPLAAALCLGSNDIVLTGEPRMKERPIGHLVDALRLGGAKITYLEQENYPPLRLQGGFTGGNVDVDGSVSSQFLTALLMTAPLTPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 44 - protein, 447 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 45 - protein, 447 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 46 - protein, 447 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 47 - protein, 447 aa, Chlamydomonas reinhardtii
  MISC_FEATURE (1)..(3): Additional amino acids
MVPVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 48 - protein, 515 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 49 - protein, 515 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 50 - protein, 515 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 51 - protein, 515 aa, Chlamydomonas reinhardtii
  MISC_FEATURE (1)..(3): Additional amino acids
MLEMQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 52 - protein, 512 aa, Chlamydomonas reinhardtii; identical to SEQ ID NO: 1

SEQ ID NO: 53 - protein, 512 aa, Chlamydomonas reinhardtii
  VARIANT (163)..(163)
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 54 - protein, 512 aa, Chlamydomonas reinhardtii
  VARIANT (252)..(252)
MQLLNQRQALRLGRSSASKNQQVAPLASRPASSLSVSASSVAPAPACSAPAGAGRRAVVVRASATKEKVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 55 - protein, 512 aa, Chlamydomonas reinhardtii; identical to SEQ ID NO: 2
  VARIANT (163)..(163)
  VARIANT (252)..(252)

SEQ ID NO: 56 - DNA, 1368 bp, Artificial Sequence
  Codon optimized sequence
atgagtcacggtgctagttcaagacctgcaacagctcgtaaaagttctggtttatctggtactgtacgtattcctggcgacaaatcaatttcacaccgttctttcatgtttggtggattagcttctggtgaaacacgtattacaggtttattagaaggagaagatgttattaatacaggtaaagcaatgcaagctatgggtgcacgtattcgtaaagagggtgacacatggattattgacggtgttggtaatggaggtttattagctcctgaagctccacttgatttcggtaatgctgctacaggttgtagattaactatgggtcttgttggagtatatgattttgattctacatttattggtgacgcatcattaacaaaacgtccaatgggtcgtgttttaaatccattacgtgaaatgggtgttcaagtaaaatcagaagacggtgaccgtttacctgtaacacttagaggtcctaaaactccaactccaattacatatcgtgtacctatggcttcagctcaagttaaatcagctgtattattagctggtttaaatacaccaggtattacaacagtaattgaaccaattatgacacgtgaccacactgagaaaatgttacaaggttttggcgctaacttaacagttgagactgatgctgatggcgtaagaacaattcgtttagaaggacgtggtaaattaactggtcaggtaatagatgtaccaggtgacccatcttctactgcttttccattagtagctgctttattagtaccaggatcagacgtaacaattttaaatgtattaatgaacccaacaagaacaggcttaatattaactttacaagaaatgggagcagatatcgaagttattaatcctcgtttagctggtggtgaagatgtagcagatttacgtgtacgttctagtactttaaaaggtgttacagttccagaagatagagcaccatcaatgattgatgaatacccaattttagctgtagcagctgcttttgctgaaggtgcaacagttatgaatggtcttgaggaattacgtgtaaaagaaagtgaccgtttatctgctgtagcaaatggcttaaaattaaatggtgttgattgtgacgaaggtgaaacatcattagtagttcgtggaagaccagatggaaaaggtttaggtaatgcttctggtgctgctgttgctacacatttagatcatcgtatagcaatgagttttttagttatgggattagttagtgaaaacccagttactgtagacgacgctacaatgattgcaacatcatttccagagtttatggacttaatggctggtttaggagctaaaattgaattaagtgatactaaagctgcataa

SEQ ID NO: 57 - DNA, 1425 bp, Synechococcus elongates
atgcgcgtagcgatcgccggtgccggacttgccggactctcctgtgccaagtacttggccgatgccggtcatacgcccatcgtctatgaacgtcgggacgtccttggcggcaaggttgccgcttggaaagatgaagacggcgactggtacgaaactggcctacatatcttttttggggcttaccccaacatgttgcagctctttaaggagctgaacattgaagatcgcctgcagtggaagtcccactcgatgatcttcaaccaacccacaaagccgggcacctattcgcgcttcgacttcccagacattccagcgccaatcaacggtgttgcagcaatcctcagcaacaacgacatgttgacctgggaagaaaaaatcaagtttggcttgggcttgttgccagcgatgattcgcggccagtcctacgtcgaagagatggatcaatactcatggacggagtggctgcgcaaacaaaatattccagagcgggtcaacgatgaagtcttcatcgccatggctaaagcgctcaactttattgacccggacgaaatttccgccacggtcgtcctaacggcactcaaccgcttcttgcaagagaagaaaggttcaatgatggcctttttggatggtgcgccgcccgagcgtctttgccagccgatcgtcgaacatgtccaagctcgcggtggtgatgtgctgctgaatgcgcctctgaaagagttcgtgctcaatgacgacagtagcgtccaagcttttcggattgctggcatcaaaggtcaagaagaacaactcattgaggcagatgcctacgtttcggcactgccggttgatccgctcaagctactgttgccggatgcatggaaagccatgccctacttccagcaactcgatggtctgcagggcgtgccggtcatcaacattcacctctggttcgatcgcaagctgaccgatatcgatcacctgctgttctcgcgatcgcccctgctcagtgtctatgccgacatgagtaacacctgtcgcgagtacgaagatcccgatcgctcaatgctagagctggtcttcgcccccgccaaagactggattggccgctccgacgaagacatcttggctgccaccatggccgagattgaaaagctattcccacagcatttcagcggtgagaatccggcacgtctgcgcaaatacaaaattgtcaaaacgcccctgtcggtctacaaagccacgccgggccgtcaacaatatcgccccgatcaagctagcccgatcgctaatttcttcctgaccggcgactacaccatgcagcgctacctcgccagtatggaaggggcggtcctatctggtaagctgacagcgcaagccatcattgctcgccaagatgagttgcaacgtcgcagcagcggacgaccgctggccgcgagtcaggcatag

SEQ ID NO: 58 - protein, 445 aa, Chlamydomonas reinhardtii
MVEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 59 - protein, 543 aa, T. viride
MVPYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWDPYRLGNTSFYGPGSSFTLDTTKKLTVVTQFETSGAINRYYVQNGVTFQQPNAELGSYSGNGLNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGDPSGGNPPGGNPPGTTTTRRPATTTGSSPGPTQSHYGQCGGIGYSGPTVCASGTTCQVLNPYYSQCLGTGENLYFQGSGGGGSDYKDDDDKGTG

SEQ ID NO: 60 - DNA, 1632 bp, Artificial Sequence
  Codon optimized sequence
atggtaccatatcgtaaacttgctgttattagtgctttcttagctactgctcgtgcacagtcagcatgtaccttacaatctgaaactcatcctccattaacatggcaaaaatgttcttcaggaggtacttgtacacaacaaactggctctgtagtaattgatgctaactggcgttggacacatgccactaatagttcaactaattgttatgacggtaatacttggtcatcaacactttgtcccgataacgaaacttgtgctaaaaattgttgtttagatggtgcagcttacgcttcaacttacggcgttactacatcaggtaactcattatcaattggtttcgtgactcaatcagcacaaaaaaatgtaggcgcacgtttatacttaatggcaagtgacacaacctatcaagaatttacattattaggtaatgagttcagtttcgacgtagatgtgagtcaattaccatgtggtttaaatggtgctctttatttcgtttcaatggacgctgatggcggtgtaagcaaatatcctactaatacagcaggtgctaaatacggaacaggctattgtgattctcagtgtcctcgtgatttaaagtttattaacggtcaagctaacgtggaaggttgggaaccaagtagtaataatgcaaatactggaattggtggtcacggatcttgttgttctgaaatggatatttgggaagctaattcaattagtgaagcattaactccacatccttgtactaccgttggccaagaaatttgtgaaggcgacggttgcggtggaacatacagtgataaccgttatggtggtacatgtgatcctgatggctgcgattgggacccatatcgtttaggaaatacatctttttatggaccaggaagttcattcacattagatacaactaaaaagttaacagttgttacacagttcgaaactagcggtgctattaatcgttattacgtgcaaaatggtgtaacttttcaacaaccaaatgcagaattaggttcttattctggtaacggccttaatgacgattattgtacagcagaagaagcagaatttggtggtagcagcttctcagataaaggtggtttaactcaattcaagaaagcaacatcaggtggtatggttttagttatgtcattatgggatgactattatgctaatatgttatggttagatagtacatatcctacaaacgaaacttcaagcactcctggtgctgttcgtggttcatgttcaacttcaagtggtgtacctgctcaagttgaaagccaaagtcctaatgcaaaagtaacttttagtaatatcaaatttggtccaattggctctacaggcgatccttcaggtggtaatccaccaggtggaaatccacctggcaccactacaacacgtcgtcctgctactaccacaggttcttctcctggaccaacacaatctcattacggtcaatgtggtggtattggttattcaggtccaactgtgtgtgcatcaggaactacatgtcaagttttaaatccatattatagccaatgtttaggtaccggtgaaaacttatactttcaaggctcaggtggcggtggaagtgattacaaagatgatgatgataaaggaaccggttaa

SEQ ID NO: 61 - protein, 683 aa, Chlamydomonas reinhardtii
MKALRSGTAVARGQAGCVSPAPRPVPMSSQTMIPSTSSPATRAPARSGRRALAVSAKLADGSRRMQSEEVRRAKEVAQAALAKDSPADWVDRYGSEPRKGADILVQALEREGVDSVFAYPGGASMEIHQALTRSDRITNVLCRHEQGEIFAAEGYAKAAGRVGVCIATSGPGATNLVTGLADAMMDSIPLVAITGQVPRRMIGTDAFQETPIVEVTRAITKHNYLVLDIKDLPRVIKEAFYLARTGRPGPVLVDVPTDIQQQLAVPDWEAPMSITGYISRLPPPVEESQVLPVVRALQGAAKPVIYYGGGCLDAQAELREFAARTGIPLASTFMGLGVVPSTDPNHLQMLGMHGTVFANYAVDQADLLVALGVRFDDRVTGKLDAFAARARIVHIDIDAAEISKNKTAHVPVCGDVKQALSHLNRLLAAEPLPADKWAGWRAELAAKRAEFPMRYPQRDDAIVPQHAIQVLGEETQGEAIITTGVGQHQMWAAQWYPYKETRRWISSGGLGSMGFGLPAALGAAVAFDGKNGRPKKTVVDIDGDGSFLMNVQELATIFIEKLDVKVMLLNNQHLGMVVQWEDRFYKANRAHTYLGKRESEWHATQDEEDIYPNFVNMAQAFGVPSRRVIVKEQLRGAIRTMLDTPGPYLLEVMVPHIEHVLPMIPGGASFKDIITEGDGTVKY

SEQ ID NO: 62 - protein, 671 aa, Chlamydomonas reinhardtii
  MISC_FEATURE (1)..(1): Additional amino acid
MAPARSGRRALAVSAKLADGSRRMQSEEVRRAKEVAQAALAKDSPADWVDRYGSEPRKGADILVQALEREGVDSVFAYPGGASMEIHQALTRSDRITNVLCRHEQGEIFAAEGYAKAAGRVGVCIATSGPGATNLVTGLADAMMDSIPLVAITGQVPRRMIGTDAFQETPIVEVTRAITKHNYLVLDIKDLPRVIKEAFYLARTGRPGPVLVDVPTDIQQQLAVPDWEAPMSITGYISRLPPPVEESQVLPVVRALQGAAKPVIYYGGGCLDAQAELREFAARTGIPLASTFMGLGVVPSTDPNHLQMLGMHGTVFANYAVDQADLLVALGVRFDDRVTGKLDAFAARARIVHIDIDAAEISKNKTAHVPVCGDVKQALSHLNRLLAAEPLPADKWAGWRAELAAKRAEFPMRYPQRDDAIVPQHAIQVLGEETQGEAIITTGVGQHQMWAAQWYPYKETRRWISSGGLGSMGFGLPAALGAAVAFDGKNGRPKKTVVDIDGDGSFLMNVQELATIFIEKLDVKVMLLNNQHLGMVVQWEDRFYKANRAHTYLGKRESEWHATQDEEDIYPNFVNMAQAFGVPSRRVIVKEQLRGAIRTMLDTPGPYLLEVMVPHIEHVLPMIPGGASFKDIITEGDGTVKYGTGDYKDDDDKSGENLYFQGHNHRHKHTG

SEQ ID NO: 63 - DNA, 2016 bp, Artificial Sequence
  Codon optimized sequence
atggctcctgctcgtagtggtagacgtgctttagctgtatctgctaaattagctgacggtagtcgtcgtatgcaatcagaggaagtaagacgtgctaaagaagttgcacaagctgcattagcaaaagattctccagctgactgggtagaccgttatggaagtgaacctcgtaaaggtgctgatattttagttcaagctttagaacgtgaaggtgtagattctgtttttgcttacccaggtggtgcttcaatggaaattcatcaggctttaacacgtagtgatcgtataactaatgttttatgtagacacgagcaaggtgaaatttttgcagctgaaggatatgctaaagctgctggtcgtgtaggtgtttgtattgctacatctggtccaggtgctactaacttagttactggtttagcagacgctatgatggattcaattcctttagttgctattactggtcaagttccacgtcgtatgattggtacagatgcatttcaagaaactccaattgtagaagtaactagagctattactaaacacaattatcttgtacttgacatcaaagacttacctcgtgtaataaaagaagcattttacttagcacgtactggccgtcctggtcctgtattagtagacgttccaactgatattcaacaacaattagctgtaccagattgggaagctcctatgtcaattacaggttatatctcaagattaccaccaccagtagaagaatcacaagttcttcctgtagttcgtgcattacaaggtgctgcaaaaccagtaatttactatggcggtggttgtttagatgctcaagcagaattacgtgaattcgctgctcgtacaggtattccattagctagtacatttatgggtttaggtgttgtaccttctacagatccaaatcatcttcaaatgttaggtatgcatggtactgtattcgctaattatgcagtagatcaagcagatttattagttgctttaggtgttagatttgatgatcgtgtaactggtaaattagacgcttttgcagctcgtgcacgtattgtacatattgatattgatgcagctgaaatatctaaaaataaaactgcacacgtacctgtatgtggtgacgttaaacaagctttaagtcatttaaatcgtttattagcagcagaaccacttcctgctgataaatgggctggttggcgtgcagaattagctgctaaacgtgctgaatttccaatgcgttatccacaaagagatgacgctattgtacctcagcatgctatccaagttttaggtgaagaaacacaaggtgaagctattattacaactggcgttggacaacatcaaatgtgggctgctcaatggtatccttataaagaaacacgtagatggattagttcaggtggtcttggtagtatgggtttcggtttacctgctgcacttggtgcagctgttgcttttgatggtaaaaatggtcgtccaaaaaaaacagttgttgatatcgatggtgatggttcattcttaatgaatgttcaagaattagctactatcttcattgaaaaattagacgtaaaagttatgcttttaaacaatcaacacttaggaatggttgttcaatgggaagaccgtttttataaagcaaatcgtgctcacacttatttaggtaaaagagaaagtgaatggcatgcaactcaagatgaagaagatatatatccaaactttgtaaatatggctcaagcattcggcgttccatcacgtcgtgtaattgtaaaagagcaattacgtggtgctattcgtactatgttagatactccaggtccatatttattagaagttatggttccacatattgaacatgttttacctatgatcccaggtggcgcttctttcaaagatattattactgaaggtgatggtactgtaaaatatggtaccggtgattacaaagacgacgacgataaatcaggtgaaaatctttactttcaaggtcataaccatagacacaaacataccggttaa

SEQ ID NO: 64 - DNA, 2016 bp, Artificial Sequence
  Codon optimized sequence
atggctcctgctcgtagtggtagacgtgctttagctgtatctgctaaattagctgacggtagtcgtcgtatgcaatcagaggaagtaagacgtgctaaagaagttgcacaagctgcattagcaaaagattctccagctgactgggtagaccgttatggaagtgaacctcgtaaaggtgctgatattttagttcaagctttagaacgtgaaggtgtagattctgtttttgcttacccaggtggtgcttcaatggaaattcatcaggctttaacacgtagtgatcgtataactaatgttttatgtagacacgagcaaggtgaaatttttgcagctgaaggatatgctaaagctgctggtcgtgtaggtgtttgtattgctacatctggtccaggtgctactaacttagttactggtttagcagacgctatgatggattcaattcctttagttgctattactggtcaagtttcacgtcgtatgattggtacagatgcatttcaagaaactccaattgtagaagtaactagagctattactaaacacaattatcttgtacttgacatcaaagacttacctcgtgtaataaaagaagcattttacttagcacgtactggccgtcctggtcctgtattagtagacgttccaactgatattcaacaacaattagctgtaccagattgggaagctcctatgtcaattacaggttatatctcaagattaccaccaccagtagaagaatcacaagttcttcctgtagttcgtgcattacaaggtgctgcaaaaccagtaatttactatggcggtggttgtttagatgctcaagcagaattacgtgaattcgctgctcgtacaggtattccattagctagtacatttatgggtttaggtgttgtaccttctacagatccaaatcatcttcaaatgttaggtatgcatggtactgtattcgctaattatgcagtagatcaagcagatttattagttgctttaggtgttagatttgatgatcgtgtaactggtaaattagacgcttttgcagctcgtgcacgtattgtacatattgatattgatgcagctgaaatatctaaaaataaaactgcacacgtacctgtatgtggtgacgttaaacaagctttaagtcatttaaatcgtttattagcagcagaaccacttcctgctgataaatgggctggttggcgtgcagaattagctgctaaacgtgctgaatttccaatgcgttatccacaaagagatgacgctattgtacctcagcatgctatccaagttttaggtgaagaaacacaaggtgaagctattattacaactggcgttggacaacatcaaatgtgggctgctcaatggtatccttataaagaaacacgtagatggattagttcaggtggtcttggtagtatgggtttcggtttacctgctgcacttggtgcagctgttgcttttgatggtaaaaatggtcgtccaaaaaaaacagttgttgatatcgatggtgatggttcattcttaatgaatgttcaagaattagctactatcttcattgaaaaattagacgtaaaagttatgcttttaaacaatcaacacttaggaatggttgttcaattagaagaccgtttttataaagcaaatcgtgctcacacttatttaggtaaaagagaaagtgaatggcatgcaactcaagatgaagaagatatatatccaaactttgtaaatatggctcaagcattcggcgttccatcacgtcgtgtaattgtaaaagagcaattacgtggtgctattcgtactatgttagatactccaggtccatatttattagaagttatggttccacatattgaacatgttttacctatgatcccaattggcgcttctttcaaagatattattactgaaggtgatggtactgtaaaatatggtaccggtgattacaaagacgacgacgataaatcaggtgaaaatctttactttcaaggtcataaccatagacacaaacataccggttaa

SEQ ID NO: 65 - protein, 671 aa, Chlamydomonas reinhardtii
  MISC_FEATURE (1)..(1): Additional amino acid
MAPARSGRRALAVSAKLADGSRRMQSEEVRRAKEVAQAALAKDSPADWVDRYGSEPRKGADILVQALEREGVDSVFAYPGGASMEIHQALTRSDRITNVLCRHEQGEIFAAEGYAKAAGRVGVCIATSGPGATNLVTGLADAMMDSIPLVAITGQVSRRMIGTDAFQETPIVEVTRAITKHNYLVLDIKDLPRVIKEAFYLARTGRPGPVLVDVPTDIQQQLAVPDWEAPMSITGYISRLPPPVEESQVLPVVRALQGAAKPVIYYGGGCLDAQAELREFAARTGIPLASTFMGLGVVPSTDPNHLQMLGMHGTVFANYAVDQADLLVALGVRFDDRVTGKLDAFAARARIVHIDIDAAEISKNKTAHVPVCGDVKQALSHLNRLLAAEPLPADKWAGWRAELAAKRAEFPMRYPQRDDAIVPQHAIQVLGEETQGEAIITTGVGQHQMWAAQWYPYKETRRWISSGGLGSMGFGLPAALGAAVAFDGKNGRPKKTVVDIDGDGSFLMNVQELATIFIEKLDVKVMLLNNQHLGMVVQLEDRFYKANRAHTYLGKRESEWHATQDEEDIYPNFVNMAQAFGVPSRRVIVKEQLRGAIRTMLDTPGPYLLEVMVPHIEHVLPMIPIGASFKDIITEGDGTVKYGTGDYKDDDDKSGENLYFQGHNHRHKHTG

SEQ ID NO: 66 - DNA, 1284 bp, Escherichia coli
atggaatccctgacgttacaacccatcgctcgtgtcgatggcactattaatctgcccggttccaagagcgtttctaaccgcgctttattgctggcggcattagcacacggcaaaacagtattaaccaatctgctggatagcgatgacgtgcgccatatgctgaatgcattaacagggttaggggtaagctatacgctttcagccgatcgtacgcgttgcgaaattatcggtaacggcggtccattacacgcagaaggtgccctggagttgttcctcggtaacgccggaacggcaatgcgtccgctggcggcagctctttgtctgggtagcaatgatattgtgctgaccggtgagccgcgtatgaaagaacgcccgattggtcatctggtggatgctctgcgcctgggcggggcgaagatcacttacctggaacaagaaaattatccgccgttgcgtttacagggcggctttaccggcggcaacgttgacgttgatggctccgtttccagccaattcctcaccgcactgttaatgactgcgcctcttgcgccggaagatacggtgattcgtattaaaggcgatctggtttctaaaccttatatcgacatcacactcaatctgatgaagacgtttggtgttgaaattgaaaatcagcactatcaacaatttgtcgtaaaaggcgggcagtcttatcagtctccgggtacttatttggtcgaaggcgatgcatcttcggcttcttactttctggcagcagcagcaatcaaaggcggcactgtaaaagtgaccggtattggacgtaacagtatgcagggtgatattcgctttgctgatgtgctggaaaaaatgggcgcgaccatttgctggggcgatgattatatttcctgcacgcgtggtgaactgaacgctattgatatggatatgaaccatattcccgatgcggcgatgaccattgccacggcggcgttatttgcaaaaggcaccaccacgctgcgcaatatctataactggcgtgttaaagaaaccgatcgcctgtttgcgatggcaacagaactgcgtaaagtcggtgcggaagtagaagaggggcacgattacattcgtatcactccaccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagcctga

SEQ ID NO: 67 - DNA, 1371 bp, Escherichia coli
  mutation (286)..(288)
  mutation (547)..(549)
  misc_feature (1282)..(1368): Affinity tag
atggaatccctgacgttacaacccatcgctcgtgtcgatggcactattaatctgcccggttccaagagcgtttctaaccgcgctttattgctggcggcattagcacacggcaaaacagtattaaccaatctgctggatagcgatgacgtgcgccatatgctgaatgcattaacagggttaggggtaagctatacgctttcagccgatcgtacgcgttgcgaaattatcggtaacggcggtccattacacgcagaaggtgccctggagttgttcctcggtaacgccgcaacggcaatgcgtccgctggcggcagctctttgtctgggtagcaatgatattgtgctgaccggtgagccgcgtatgaaagaacgcccgattggtcatctggtggatgctctgcgcctgggcggggcgaagatcacttacctggaacaagaaaattatccgccgttgcgtttacagggcggctttaccggcggcaacgttgacgttgatggctccgtttccagccaattcctcaccgcactgttaatgactgcgcctcttacgccggaagatacggtgattcgtattaaaggcgatctggtttctaaaccttatatcgacatcacactcaatctgatgaagacgtttggtgttgaaattgaaaatcagcactatcaacaatttgtcgtaaaaggcgggcagtcttatcagtctccgggtacttatttggtcgaaggcgatgcatcttcggcttcttactttctggcagcagcagcaatcaaaggcggcactgtaaaagtgaccggtattggacgtaacagtatgcagggtgatattcgctttgctgatgtgctggaaaaaatgggcgcgaccatttgctggggcgatgattatatttcctgcacgcgtggtgaactgaacgctattgatatggatatgaaccatattcccgatgcggcgatgaccattgccacggcggcgttatttgcaaaaggcaccaccacgctgcgcaatatctataactggcgtgttaaagaaaccgatcgcctgtttgcgatggcaacagaactgcgtaaagtcggtgcggaagtagaagaggggcacgattacattcgtatcactccaccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagccggtaccggtgattacaaagacgacgacgataaatcaggtgaaaatctttactttcaaggtcataaccatagacacaaacataccggttga

SEQ ID NO: 68 - DNA, 1284 bp, Artificial Sequence
  Codon optimized sequence
atggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctggtactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttagcacctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgcttaa

SEQ ID NO: 69 - protein, 427 aa, Chlamydomonas reinhardtii
MESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 70 - DNA, 1284 bp, Artificial Sequence
  Codon optimized sequence
atggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctgcaactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttagcacctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgcttaa

SEQ ID NO: 71 - protein, 427 aa, Chlamydomonas reinhardtii
  VARIANT (96)..(96)
MESLTLQPIARVDGTINLPGSKSVSNRALLLAALAHGKTVLTNLLDSDDVRHMLNALTALGVSYTLSADRTRCEIIGNGGPLHAEGALELFLGNAATAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 72 - DNA, 1284 bp, Artificial Sequence
  Codon optimized sequence
atggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctggtactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttaacgcctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgcttaa

SEQ ID NO: 73 - protein, 427 aa, Chlamydomonas reinhardtii
  VARIANT (183)..(183)
MESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLTPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 74 - DNA, 1284 bp, Artificial Sequence
  Codon optimized sequence
atggaaagtttaacacttcaaccaattgctagagttgatggtactattaacttacctggttcaaaatctgtatctaaccgtgcacttttattagctgcattagcacatggaaaaactgtattaacaaatcttttagactcagatgatgtacgtcacatgttaaacgcattaactgcattaggtgtatcatatactctttctgctgatcgtactcgttgcgaaatcattggaaatggaggtccattacacgcagaaggcgctttagaacttttcttaggtaacgctgcaactgctatgcgtccattagcagctgctttatgtttaggtagtaacgatattgttttaactggagaaccacgtatgaaagaacgtcctattggacacttagtagatgctttacgtttaggaggtgctaaaattacatatcttgaacaagaaaactatcctccattacgtttacagggtggttttactggtggtaacgttgatgttgatggtagtgtttcttctcaattcttaactgctcttttaatgacagctcctttaacgcctgaggatacagttattcgtattaaaggtgatcttgttagtaaaccttatattgacattacattaaacttaatgaaaacatttggtgttgaaattgaaaaccagcactaccagcagtttgtagttaaaggtggacaaagttaccaatctcctggtacttatttagttgaaggcgatgcatcaagtgcttcatactttttagcagctgcagctattaaaggtggtacagttaaagttacaggcattggtcgtaacagtatgcaaggtgatattagatttgcagatgttttagagaaaatgggtgctactatttgctggggtgacgactatatcagttgcactcgtggtgaacttaatgctattgatatggatatgaatcacattccagatgcagctatgacaattgcaacagcagcattatttgctaaaggaactacaacacttcgtaatatctataattggcgtgttaaagaaacagatcgtttattcgcaatggctactgaacttcgtaaagttggtgctgaagtagaggaaggtcacgattatattcgtattactcctcctgagaaattaaacttcgctgaaattgcaacatataacgatcaccgtatggctatgtgtttttcattagttgctttaagtgatactcctgttacaattttagaccctaaatgtacagctaaaacattccctgactattttgaacaattagctcgtatttctcaggctgcttaa

SEQ ID NO: 75 - protein, 427 aa, Chlamydomonas reinhardtii
  VARIANT (96)..(96)
  VARIANT (183)..(183)
MESLTLQPIARVDGTINLPGSKSVSNRALLLAALAHGKTVLTNLLDSDDVRHMLNALTALGVSYTLSADRTRCEIIGNGGPLHAEGALELFLGNAATAMRPLAAALCLGSNDIVLTGEPRMKERPIGHLVDALRLGGAKITYLEQENYPPLRLQGGFTGGNVDVDGSVSSQFLTALLMTAPLTPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 76 - DNA, 1335 bp, Artificial Sequence
  Codon optimized sequence
gtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctggaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttgctgttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacactaa

SEQ ID NO: 77 - protein, 444 aa, Chlamydomonas reinhardtii
VEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 78 - DNA, 1335 bp, Artificial Sequence
  Codon optimized sequence
gtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctgcaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttgctgttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacactaa

SEQ ID NO: 79 - protein, 444 aa, Chlamydomonas reinhardtii
  VARIANT (95)..(95)
VEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLAVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 80 - DNA, 1335 bp, Artificial Sequence
  Codon optimized sequence
gtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctggaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttacggttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacactaa

SEQ ID NO: 81 - protein, 444 aa, Chlamydomonas reinhardtii
  VARIANT (184)..(184)
VEELTIQPVKKIAGTVKLPGSKSLSNRILLLAALSEGTTLVKNLLDSDDIRYMVGALKALNVKLEENWEAGEMVVHGCGGRFDSAGAELFLGNAGTAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 82 - DNA, 1335 bp, Artificial Sequence
  Codon optimized sequence
gtagaagaacttacaattcaacctgtaaaaaaaattgcaggaactgttaaattacctggttcaaaatctttatctaatcgtattttattacttgctgctttatctgaaggtactacattagttaaaaacttacttgacagtgatgatattagatatatggtaggagcattaaaagcattaaatgttaaattagaagaaaactgggaagctggtgaaatggtagtacacggctgtggtggtcgttttgattcagcaggtgcagagttatttcttggcaacgctgcaactgctatgcgtcctttaacagctgctgttgttgcagctggtagaggtaaatttgttttagatggtgttgctcgtatgcgtgaacgtccaattgaagaccttgttgacggtttagttcagcttggagttgatgcaaaatgtacaatgggtacaggttgtcctccagttgaagtaaacagtaaaggtttaccaacaggtaaagtttacttaagtggtaaagtaagttcacagtatttaacagctcttttaatggcagcaccacttacggttcctggtggtgctggtggtgacgctatcgaaattatcattaaagatgaattagtttctcaaccttacgttgatatgacagttaaattaatggaacgttttggtgtagtagttgaacgtttaaatggattacaacatttaagaataccagcaggacaaacttataaaactcctggtgaagcatacgttgaaggtgatgctagtagtgctagttactttttagctggtgctacaattacaggtggtacagtaacagttgaaggttgtggtagtgattcattacaaggtgacgtacgtttcgctgaagtaatgggattattaggagctaaagtagagtggtcaccttattctattactattactggcccaagtgctttcggtaaaccaattactggaatagaccacgattgtaatgatattccagatgctgctatgactttagctgttgcagcattatttgctgatcgtcctacagcaattagaaacgtttataactggcgtgttaaagaaactgaacgtatggtagctattgtaacagagttaagaaaattaggtgcagaagttgaagaaggaagagattactgtattgttacacctcctcctggaggcgtaaaaggcgttaaagcaaatgttggcattgatacttacgatgatcaccgtatggctatggctttctctcttgtagcagcagcaggtgttcctgtagttattcgtgaccctggttgtactcgtaaaacatttccaacatacttcaaagtatttgaatcagttgctcaacactaa

SEQ ID NO: 83 - protein, 444 aa, Chlamydomonas reinhardtii
  VARIANT (95)..(95)
  VARIANT (184)..(184)
VEELTIQPVKKIAGTVKLPGSKSLSNRILLLAALSEGTTLVKNLLDSDDIRYMVGALKALNVKLEENWEAGEMVVHGCGGRFDSAGAELFLGNAATAMRPLTAAVVAAGRGKFVLDGVARMRERPIEDLVDGLVQLGVDAKCTMGTGCPPVEVNSKGLPTGKVYLSGKVSSQYLTALLMAAPLTVPGGAGGDAIEIIIKDELVSQPYVDMTVKLMERFGVVVERLNGLQHLRIPAGQTYKTPGEAYVEGDASSASYFLAGATITGGTVTVEGCGSDSLQGDVRFAEVMGLLGAKVEWSPYSITITGPSAFGKPITGIDHDCNDIPDAAMTLAVAALFADRPTAIRNVYNWRVKETERMVAIVTELRKLGAEVEEGRDYCIVTPPPGGVKGVKANVGIDTYDDHRMAMAFSLVAAAGVPVVIRDPGCTRKTFPTYFKVFESVAQH

SEQ ID NO: 84 - DNA, 1539 bp, Chlamydomonas reinhardtii
atgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactag

SEQ ID NO: 85 - protein, 512 aa, Chlamydomonas reinhardtii; identical to SEQ ID NO: 1

SEQ ID NO: 86 - DNA, 1539 bp, Chlamydomonas reinhardtii
  mutation (487)..(489)
atgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactag

SEQ ID NO: 87 - protein, 512 aa, Chlamydomonas reinhardtii; identical to SEQ ID NO: 53
  VARIANT (163)..(163)

SEQ ID NO: 88 - DNA, 1539 bp, Chlamydomonas reinhardtii
  mutation (754)..(756)
atgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactag

SEQ ID NO: 89 - protein, 512 aa, Chlamydomonas reinhardtii; identical to SEQ ID NO: 54
  VARIANT (252)..(252)

SEQ ID NO: 90 - DNA, 1539 bp, Chlamydomonas reinhardtii
  mutation (487)..(489)
  mutation (754)..(756)
atgcagctcctcaaccagcgtcaggccctgcgcctgggccgctcttctgctagcaagaaccagcaggttgctcctctggcctctcgccctgcgtcttccttgagcgtcagcgcctccagcgtcgcgccggcgcctgcttgcagtgctcccgcgggcgcaggtcgccgcgctgttgtcgtgcgcgcttcagctaccaaggagaaggtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcggatacccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccgccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactag

SEQ ID NO: 91 - protein, 512 aa, Chlamydomonas reinhardtii; identical to SEQ ID NO: 2
  VARIANT (163)..(163)
  VARIANT (252)..(252)

SEQ ID NO: 92 - DNA, 4122 bp, Chlamydomonas reinhardtii
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactga

SEQ ID NO: 93 - DNA, 4122 bp, Chlamydomonas reinhardtii
  mutation (899)..(901)
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctggcggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactga

SEQ ID NO: 94 - DNA, 4122 bp, Chlamydomonas reinhardtii
  mutation (2203)..(2205)
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccggcacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactga

SEQ ID NO: 95 - DNA, 4122 bp, Chlamydomonas reinhardtii
  mutation (899)..(901)
  mutation (2203)..(2205)
atgcagctcctcaaccagcgtcaggccctgcgcctgggccgctcttctgctagcaagaaccagcaggttgctcctctggcctctcgccctgcgtcttccttgagcgtcagcgcctcgagcgtcgcgccggcgcctgcttgcagtgctcccgcgggcgcaggtcgccgcgctgttgtcgtgcgcgcttcagctaccaaggagaagggtgaggcgaaattggcaattggggatccccccaaaacgtgacctgcttgcaaccagcacaacaactttcgcatgcacatcgtgatggcttcgcagtggaggagctgaccatccagcccgtgaagaagatcgcgggcactgtgaaactgcccggctcgaagtctctgtcgaaccgcatcctgctgctggcggccctttcggagggcaccacgctagtgaagaacctgctggtgcgtggggcccaggggacgttagggcaacgctacgggggcagcatagacaactacgcaggccggcactcgggcgagcgagaacatggatgcacgtaaagctaggggccatgcagggaagagcttgctagcggcaagggagggcacggtagcgccggtacagctggcccaggcccagtgctgtgaatgaccctggcctccgccgacacgccctggcaggctgctactcggtcctgccgccatccaccctcccacccacacccacacacatgcacagtcctgctctcttatttacacttgtacacatgcgcacacaggacagcgatgacatccgctacatggtgggcgcgctgaaggcgctgaacgtcaagcttgaggagaactgggaggcgggcgagatggtggtgcacggctgcggcggccgcttcgacagcgccggcgccgagctgttcctgggcaacgccgcaacggccatgcgcccgctcacggcagcggtggtggcggccggccgcggcaagtgagtgggggcgatacatggggatggtggtgggtgtgaggtggtggaagggtggtggcaggagcggcgggcagcgttgagggtaggcgggtgttgatctggaggacaggggctggacaggggcagagtcaggagtttctcaggcggagcaagcggtacagcggctggcagacagcaacggggccaggacggcactgcctgctgctcgcggacactactgcgcagatgggctggcacgcccctgattgcaccagccccatgccacgtgcacacgtagcagctccgttgagtcgtgcgcccccgcacctgcgcgggcacgctgcctactcctaacccgttgcctccaccctcctcgggacccttccctcccctctacgccgccaggttcgtgctggacggtgttgcccgcatgcgcgagcggcccattgaggacctggtggacgggctggtgcagctgggcgtggacgccaagtgcaccatgggcactggctgcccgcccgtggaggtcaacagcaaggggctgcccaccggcaaggtgggcgccgggtcgggcagagggggcggcggtaaagggggcgcggggggggcggcttatgggagggcgagcgggggttagtggtggggctggaggggtggacgggcaagtccattccaaatgacgctggcaggcaagcggcccgccaaccccctgcgttatgccacgcggtcaaagcagttctggggagagcgtgggaatgcaagcagaggggaagggacccagaggccatcaacggaagtgctgtacggaagctgaggtcaacacagcctggcggtcagggcaaagggaggcgatggctagccgtgagcggtcacgggggtgtccagggaagtgacagcgctgtcggctgcaagccagtcacatttggcattcaaggacagctgcagagggccgcagcctttggagggtcggaggctactgcagggaccagcgtgggaggctgggggccacttgtacaagtgcctacccgtcctgtccaagcctggatacatatacccggggaaccgtgcgctacaccactaccggtagtttcaatcccgtgtttcacagactgctacccccaccccaccccaagatcgcctaccgtctaccacttaacgtatcatagatgtaaccccaccccatgaatggctaccccaatcccactgcaggtgtacctgtccggcaaggtgtccagccagtacctgacggcgctgctcatggcggcgccgctgacggtgccgggcggcgcgggcggcgacgctatcgagatcatcatcaaggacgagctggtgtcgcagccgtatgtggacatgaccgtcaagctcatggagcggttcggggtggtggtggagcggctcaacggcctgcagcacctgcgggtgggtgcgggcgtggctgagtgtcctgtggggtgtgtgtgtatgtcggggatggggatttgcagcggtaaataaatgttgatgagggtggggtggggtctggggtgttggtaccagcatttcttcgtatgatgtgggtcaaaggagggccggggcttcagacaatgcccaacccatatcacctgggccgggtgctggacggtgactgtagaggcagaggggagcggaggggcagcgtagcctaaaagaagcggatggaaggggtcagcgcggccgaacctgcggctgtgcttcaggcagccagcagggtggtgtcggtggcgctggggcgtggaaagacgatgactgcgccgatgccccttcctctcatgccctcaatcctgtgtcaccattctcgccccccccccccggacatcggtaaaaacgcgtttgctgtactacggtgcctggctacgtcttcacgtgttcatgaatgtaaccccccggttcgttccctgccccacagatccccgccggccagacgtacaagacccctggagaggcgtacgtggagggcgacgcctcctctgcctcctacttcctggcgggcgccacaatcaccggcggcaccgtcaccgtggagggctgcggcagcgacagcctgcagggagacgtgcgcttcgccgaggtgcggactggaggaggcggcgggacgtggcatgtgtgttcggggcggcagcggcagcgccggcggcggcggggaggggcagaaaaggcggcttgggccctgggacgtgtggtggaggggctgaaggggaagtgggttggcttggcaccgtacgccggtatgcgctgactctttgcgctgacgtgtgtgacgcctgtgcgtgtgcgtgtgcccccacaggtcatgggtctgctgggcgccaaggtggagtggtcgccttactccatcaccatcaccggcccctccgccttcggcaagcccatcaccggcatcgaccacgactgcaacgacatcccggacgccgccatgacactggccgtggccgcgctgttcgccgaccggtgcgtggcgcttggcgttcttggcggttgggcggggcatggagcggcctggtcgggggggttgctgcgacaccgcggtttggtattcgtctcttctcagctcaagagcgttgactccaacacccattcgcatcgctgtcgccgctgtgactgctgacgccaccgtcgtccccgcgccacccgccaaccccctgctccgccctgcctcaccgcttgcccgcagccccaccgccatccgcaacgtgtacaactggcgtgtgaaggagacggagcgcatggtggccattgtgacggagctgcgcaagctgggcgcggaggtggaggagggccgcgactactgcatcgtcacgccgcctccgggtgggtgcaggagcgcgcagtaacacggggtacacggggtggcagacgggcacagggggcccaggagggcatgaggtggtggcgcctgttgaggttggggtttgctggcccggggacctgtttgctgggctcgggcatgtgatcctcccccctcctcccgctgcttctgctcctgtccctgctgcaggtggtgtcaagggcgtcaaggccaacgtgggcatcgacacctacgacgaccaccgcatggccatggccttctcgctggtggcggccgccggcgtgcccgtggtcatccgcgatcccggctgcacgcggaagaccttccccacctacttcaaggtgttcgagagcgtggcgcagcactga

SEQ ID NO: 96 - protein, 641 aa, Chlamydomonas reinhardtii
APARSGRRALAVSAKLADGSRRMQSEEVRRAKEVAQAALAKDSPADWVDRYGSEPRKGADILVQALEREGVDSVFAYPGGASMEIHQALTRSDRITNVLCRHEQGEIFAAEGYAKAAGRVGVCIATSGPGATNLVTGLADAMMDSIPLVAITGQVPRRMIGTDAFQETPIVEVTRAITKHNYLVLDIKDLPRVIKEAFYLARTGRPGPVLVDVPTDIQQQLAVPDWEAPMSITGYISRLPPPVEESQVLPVVRALQGAAKPVIYYGGGCLDAQAELREFAARTGIPLASTFMGLGVVPSTDPNHLQMLGMHGTVFANYAVDQADLLVALGVRFDDRVTGKLDAFAARARIVHIDIDAAEISKNKTAHVPVCGDVKQALSHLNRLLAAEPLPADKWAGWRAELAAKRAEFPMRYPQRDDAIVPQHAIQVLGEETQGEAIITTGVGQHQMWAAQWYPYKETRRWISSGGLGSMGFGLPAALGAAVAFDGKNGRPKKTVVDIDGDGSFLMNVQELATIFIEKLDVKVMLLNNQHLGMVVQWEDRFYKANRAHTYLGKRESEWHATQDEEDIYPNFVNMAQAFGVPSRRVIVKEQLRGAIRTMLDTPGPYLLEVMVPHIEHVLPMIPGGASFKDIITEGDGTVKY

SEQ ID NO: 97 - DNA, 1929 bp, Artificial Sequence
  Codon optimized sequence
atggctcctgctcgtagtggtagacgtgctttagctgtatctgctaaattagctgacggtagtcgtcgtatgcaatcagaggaagtaagacgtgctaaagaagttgcacaagctgcattagcaaaagattctccagctgactgggtagaccgttatggaagtgaacctcgtaaaggtgctgatattttagttcaagctttagaacgtgaaggtgtagattctgtttttgcttacccaggtggtgcttcaatggaaattcatcaggctttaacacgtagtgatcgtataactaatgttttatgtagacacgagcaaggtgaaatttttgcagctgaaggatatgctaaagctgctggtcgtgtaggtgtttgtattgctacatctggtccaggtgctactaacttagttactggtttagcagacgctatgatggattcaattcctttagttgctattactggtcaagttccacgtcgtatgattggtacagatgcatttcaagaaactccaattgtagaagtaactagagctattactaaacacaattatcttgtacttgacatcaaagacttacctcgtgtaataaaagaagcattttacttagcacgtactggccgtcctggtcctgtattagtagacgttccaactgatattcaacaacaattagctgtaccagattgggaagctcctatgtcaattacaggttatatctcaagattaccaccaccagtagaagaatcacaagttcttcctgtagttcgtgcattacaaggtgctgcaaaaccagtaatttactatggcggtggttgtttagatgctcaagcagaattacgtgaattcgctgctcgtacaggtattccattagctagtacatttatgggtttaggtgttgtaccttctacagatccaaatcatcttcaaatgttaggtatgcatggtactgtattcgctaattatgcagtagatcaagcagatttattagttgctttaggtgttagatttgatgatcgtgtaactggtaaattagacgcttttgcagctcgtgcacgtattgtacatattgatattgatgcagctgaaatatctaaaaataaaactgcacacgtacctgtatgtggtgacgttaaacaagctttaagtcatttaaatcgtttattagcagcagaaccacttcctgctgataaatgggctggttggcgtgcagaattagctgctaaacgtgctgaatttccaatgcgttatccacaaagagatgacgctattgtacctcagcatgctatccaagttttaggtgaagaaacacaaggtgaagctattattacaactggcgttggacaacatcaaatgtgggctgctcaatggtatccttataaagaaacacgtagatggattagttcaggtggtcttggtagtatgggtttcggtttacctgctgcacttggtgcagctgttgcttttgatggtaaaaatggtcgtccaaaaaaaacagttgttgatatcgatggtgatggttcattcttaatgaatgttcaagaattagctactatcttcattgaaaaattagacgtaaaagttatgcttttaaacaatcaacacttaggaatggttgttcaatgggaagaccgtttttataaagcaaatcgtgctcacacttatttaggtaaaagagaaagtgaatggcatgcaactcaagatgaagaagatatatatccaaactttgtaaatatggctcaagcattcggcgttccatcacgtcgtgtaattgtaaaagagcaattacgtggtgctattcgtactatgttagatactccaggtccatatttattagaagttatggttccacatattgaacatgttttacctatgatcccaggtggcgcttctttcaaagatattattactgaaggtgatggtactgtaaaatattaa

SEQ ID NO: 98 - DNA, 1929 bp, Artificial Sequence
  Codon optimized sequence
atggctcctgctcgtagtggtagacgtgctttagctgtatctgctaaattagctgacggtagtcgtcgtatgcaatcagaggaagtaagacgtgctaaagaagttgcacaagctgcattagcaaaagattctccagctgactgggtagaccgttatggaagtgaacctcgtaaaggtgctgatattttagttcaagctttagaacgtgaaggtgtagattctgtttttgcttacccaggtggtgcttcaatggaaattcatcaggctttaacacgtagtgatcgtataactaatgttttatgtagacacgagcaaggtgaaatttttgcagctgaaggatatgctaaagctgctggtcgtgtaggtgtttgtattgctacatctggtccaggtgctactaacttagttactggtttagcagacgctatgatggattcaattcctttagttgctattactggtcaagtttcacgtcgtatgattggtacagatgcatttcaagaaactccaattgtagaagtaactagagctattactaaacacaattatcttgtacttgacatcaaagacttacctcgtgtaataaaagaagcattttacttagcacgtactggccgtcctggtcctgtattagtagacgttccaactgatattcaacaacaattagctgtaccagattgggaagctcctatgtcaattacaggttatatctcaagattaccaccaccagtagaagaatcacaagttcttcctgtagttcgtgcattacaaggtgctgcaaaaccagtaatttactatggcggtggttgtttagatgctcaagcagaattacgtgaattcgctgctcgtacaggtattccattagctagtacatttatgggtttaggtgttgtaccttctacagatccaaatcatcttcaaatgttaggtatgcatggtactgtattcgctaattatgcagtagatcaagcagatttattagttgctttaggtgttagatttgatgatcgtgtaactggtaaattagacgcttttgcagctcgtgcacgtattgtacatattgatattgatgcagctgaaatatctaaaaataaaactgcacacgtacctgtatgtggtgacgttaaacaagctttaagtcatttaaatcgtttattagcagcagaaccacttcctgctgataaatgggctggttggcgtgcagaattagctgctaaacgtgctgaatttccaatgcgttatccacaaagagatgacgctattgtacctcagcatgctatccaagttttaggtgaagaaacacaaggtgaagctattattacaactggcgttggacaacatcaaatgtgggctgctcaatggtatccttataaagaaacacgtagatggattagttcaggtggtcttggtagtatgggtttcggtttacctgctgcacttggtgcagctgttgcttttgatggtaaaaatggtcgtccaaaaaaaacagttgttgatatcgatggtgatggttcattcttaatgaatgttcaagaattagctactatcttcattgaaaaattagacgtaaaagttatgcttttaaacaatcaacacttaggaatggttgttcaattagaagaccgtttttataaagcaaatcgtgctcacacttatttaggtaaaagagaaagtgaatggcatgcaactcaagatgaagaagatatatatccaaactttgtaaatatggctcaagcattcggcgttccatcacgtcgtgtaattgtaaaagagcaattacgtggtgctattcgtactatgttagatactccaggtccatatttattagaagttatggttccacatattgaacatgttttacctatgatcccaattggcgcttctttcaaagatattattactgaaggtgatggtactgtaaaatattaa

SEQ ID NO: 99 - protein, 641 aa, Chlamydomonas reinhardtii
  VARIANT (156)..(156)
  VARIANT (538)..(538)
  VARIANT (624)..(624)
APARSGRRALAVSAKLADGSRRMQSEEVRRAKEVAQAALAKDSPADWVDRYGSEPRKGADILVQALEREGVDSVFAYPGGASMEIHQALTRSDRITNVLCRHEQGEIFAAEGYAKAAGRVGVCIATSGPGATNLVTGLADAMMDSIPLVAITGQVSRRMIGTDAFQETPIVEVTRAITKHNYLVLDIKDLPRVIKEAFYLARTGRPGPVLVDVPTDIQQQLAVPDWEAPMSITGYISRLPPPVEESQVLPVVRALQGAAKPVIYYGGGCLDAQAELREFAARTGIPLASTFMGLGVVPSTDPNHLQMLGMHGTVFANYAVDQADLLVALGVRFDDRVTGKLDAFAARARIVHIDIDAAEISKNKTAHVPVCGDVKQALSHLNRLLAAEPLPADKWAGWRAELAAKRAEFPMRYPQRDDAIVPQHAIQVLGEETQGEAIITTGVGQHQMWAAQWYPYKETRRWISSGGLGSMGFGLPAALGAAVAFDGKNGRPKKTVVDIDGDGSFLMNVQELATIFIEKLDVKVMLLNNQHLGMVVQLEDRFYKANRAHTYLGKRESEWHATQDEEDIYPNFVNMAQAFGVPSRRVIVKEQLRGAIRTMLDTPGPYLLEVMVPHIEHVLPMIPIGASFKDIITEGDGTVKY

SEQ ID NO: 100 - DNA, 1284 bp, Escherichia coli
  mutation (286)..(288)
  mutation (547)..(549)
atggaatccctgacgttacaacccatcgctcgtgtcgatggcactattaatctgcccggttccaagagcgtttctaaccgcgctttattgctggcggcattagcacacggcaaaacagtattaaccaatctgctggatagcgatgacgtgcgccatatgctgaatgcattaacagggttaggggtaagctatacgctttcagccgatcgtacgcgttgcgaaattatcggtaacggcggtccattacacgcagaaggtgccctggagttgttcctcggtaacgccgcaacggcaatgcgtccgctggcggcagctctttgtctgggtagcaatgatattgtgctgaccggtgagccgcgtatgaaagaacgcccgattggtcatctggtggatgctctgcgcctgggcggggcgaagatcacttacctggaacaagaaaattatccgccgttgcgtttacagggcggctttaccggcggcaacgttgacgttgatggctccgtttccagccaattcctcaccgcactgttaatgactgcgcctcttacgccggaagatacggtgattcgtattaaaggcgatctggtttctaaaccttatatcgacatcacactcaatctgatgaagacgtttggtgttgaaattgaaaatcagcactatcaacaatttgtcgtaaaaggcgggcagtcttatcagtctccgggtacttatttggtcgaaggcgatgcatcttcggcttcttactttctggcagcagcagcaatcaaaggcggcactgtaaaagtgaccggtattggacgtaacagtatgcagggtgatattcgctttgctgatgtgctggaaaaaatgggcgcgaccatttgctggggcgatgattatatttcctgcacgcgtggtgaactgaacgctattgatatggatatgaaccatattcccgatgcggcgatgaccattgccacggcggcgttatttgcaaaaggcaccaccacgctgcgcaatatctataactggcgtgttaaagaaaccgatcgcctgtttgcgatggcaacagaactgcgtaaagtcggtgcggaagtagaagaggggcacgattacattcgtatcactccaccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagcctga